(19) United States
(12) Patent Application Publication
(10) Pub. No.: US 2025/0066732 A1
(43) Pub. Date: Feb. 27, 2025

(54) AMNIOTIC FLUID CELL-DERIVED EXTRACELLULAR MATRIX AND USES THEREOF

(71) Applicant: STEMBIOSYS, INC., San Antonio, TX (US)

(72) Inventors: Travis BLOCK, San Antonio, TX (US); Edward S. GRIFFEY, San Antonio, TX (US); Mary M. NAVARRO, San Antonio, TX (US)

(73) Assignee: STEMBIOSYS, INC., San Antonio, TX (US)

(21) Appl. No.: 18/947,876

(22) Filed: Nov. 14, 2024

Related U.S. Application Data
(63) Continuation of application No. 17/500,408, filed on Oct. 13, 2021.
Application No. 17/500,408 is a continuation of application No. 16/592,539, filed on Oct. 3, 2019, now Pat. No. 11,180,732.
(60) Provisional application No. 62/740,817, filed on Oct. 3, 2018.

(51) Int. Cl. C12N 5/077; C12N 5/074; C12N 5/0775

(52) U.S. Cl. CPC C12N 5/0655; C12N 5/0663; C12N 5/0696; C12N 2533/52; C12N 2533/90

(57) ABSTRACT

Disclosed is a cell-derived extracellular matrix (ECM) derived in vitro from cells isolated from amniotic fluid, and methods of use for the isolation, maintenance, and proliferation of adherent cells including stem cells, as well as for the differentiation of stem cells.

### CROSS REFERENCE TO RELATED APPLICATIONS

This application is a continuation of U.S. patent application Ser. No. 17/500,408, filed Oct. 13, 2021, which is a continuation of U.S. patent application Ser. No. 16/592,539 filed Oct. 3, 2019, which claims the benefit of U.S. Provisional Patent Application No. 62/740,817 filed Oct. 3, 2018. The contents of each of the referenced applications are incorporated into the present application by reference.

### FIELD OF THE INVENTION

The invention generally relates to cell-derived extracellular matrices and uses thereof including isolation, maintenance, and proliferation of mammalian cells and differentiation of stem cells.

### BACKGROUND

In vitro cell culture is perhaps the most ubiquitous, important, and poorly understood aspect of all cell biology as well as the developing fields of regenerative medicine and tissue engineering. Firstly, it allows for the observation of cell behavior so that various aspects of cell function may be studied in detail. Secondly, it allows for increase in numbers of specific cell groups. For basic research, as well as many clinical applications, it is necessary to achieve large quantities of relatively rare cells from small biological samples. In vitro cell culture permits small numbers of cells to be expanded outside the body to achieve more relevant numbers. Lastly, it permits the storage of cells for later use. By expanding cell numbers in vitro, and freezing viable cells for later use, relatively small biological samples can yield cells for multiple experiments over the span of days, months, or even years.

Despite the omnipresence of cell culture, the effects that in vitro culture has on the native characteristics of the cells it is still relatively poorly understood. Many of our current practices have arisen not from deliberate thought, planning, and experimentation, but instead from chance observations. Mammalian cell culture began in the early 1900s when, in 1911, Alexis Carrel and Montrose Burrows first published an academic paper on the cultivation of mammalian tissues in vitro. They were studying the physiology and anatomy of tissues by cutting sections of mammalian tissues and placing them on microscope slides. They then noticed that some cells migrated out of the tissue onto the slide. They went on to describe techniques for culturing cells in perpetuity. It now appears that some of their observations may not have been valid, but their work paved the way for modern cell culture.

After the discovery of hematopoietic stem cells (HSCs), groups all over the world were studying (HSCs). During their culture (in suspension), it was observed that a sub-population of bone marrow cells stuck to the bottom of the plastic flasks and began to proliferate. These cells were later recognized to be distinct from HSCs, and were eventually dubbed mesenchymal stem cells (MSCs). Because of this chance observation that lead to the discovery of MSCs, plastic adherence is still widely used as a defining attribute of MSCs and many other mammalian cell types.

The practice of culturing cells on plastic substrates is problematic because there is substantial evidence, that is now widely accepted in the literature, demonstrating the critical role of the microenvironment in regulating cell function. The microenvironment has been shown to help direct the differentiation of stem and progenitor cells, and regulate the behavior of mature cell types.

When cells are removed from their native environment to be expanded in vitro they lose important cues from their surrounding extracellular matrix or microenvironment which relay important information to the cells regarding the composition and state of their surroundings. Changes to a cell's microenvironment have a profound effect on the behavior of those cells. The current standard for isolation and expansion of most adherent cells in vitro is to place the cells in culture vessels composed of polystyrene (plastic). The polystyrene may have been treated in some manner to facilitate cell attachment and growth but the surface is, in most cases, completely foreign to the cell. In other cases, the surface may be coated with individual matrix proteins (e.g. fibronectin or collagen) or some combination of proteins. These simple substrates disregard the complexity of the native microenvironment as well as the critical role of the microenvironment in normal cell function. The cell will immediately begin to respond to this foreign environment in a manner that is much different than when the cell is in its native environment.

Five major approaches are currently employed to address this issue of culturing cells on plastic substrates:
1. Ignore the problem.—Instead of trying to achieve a desired function that matches what would be expected in vivo, a multitude of cell types can be tested in various media in order to find cells that will exhibit a specific desired function without the appropriate matrix substrate. This approach is unsophisticated and often fails to produce desired results because of the complex interplay of variables and the breadth of interactions between cells and the extracellular matrix.
2. Identify key components.—Many academic laboratories and several companies have taken the approach of considering the tissue from which cells are isolated and looking for unique elements of that tissue that may be important for cell function. Cells are then cultured on simple substrates consisting of only one or a few matrix components. This approach often fails because matrices are naturally very complex environments including over one-hundred different proteins in some cases. Cells respond just as strongly to signals they need and fail to receive, as to signals they do not need and do receive.
3. Shotgun approach—The use of protein gels like MATRIGEL™ employs a sort of shotgun approach. A gel is created that contains many different matrix proteins with the hopes that it will contain the necessary binding motifs for many different cell types. This approach may fail by providing cues that push cells in a particular direction or by failing to provide all the cues that cells are expecting.
4. Tissue-derived matrices—This is a biomimetic approach that typically involves isolating a tissue of interest from a genetically similar animal, physically disrupting or chemically digesting the tissue to obtain a solution or uniform suspension, and then coating culture vessels with the deconstructed tissue. For example, someone who wishes to culture satellite cells, might collect muscle, homogenize the tissue, and then coat a culture vessel in homogenized muscle prior to seeding the cells. This method often fails for a few key reasons. Firstly, even within a specific tissue type, the stem cell/progenitor cell niche, may be distinct from the rest of the tissue. Simply homogenizing muscle does not guarantee that an appropriate niche is being created. Secondly, the niche consists of structural and physical cues, in addition to biochemical cues. Even if many/most of the biochemical cues are present in a tissue homogenate, the structure has been destroyed, and cells may sense very different mechanical cues. Lastly, manufacturability of tissue derived matrices is dependent on availability of tissues. This affects the total amount of cell culture possible and contributes to lot-to-lot variability.
5. Cell-derived matrices—Cells in culture can be induced to secrete a matrix in their culture vessel. This matrix is the best approximation available of the in vivo niche, and can be manufactured in vitro. Cells can be induced in vitro to elaborate a matrix and then the cells can subsequently be eliminated from the matrix, for example by using non-ionizing detergent to retain structure and chemistry of the matrix. This approach has several key advantages. (1) The matrix structure can be recreated and left undisturbed. (2) The matrix can be customized based on tissue/cell type of interest. (3) The matrix can be specific to stem and progenitor cell niche. (4) The matrix can be manufactured in large quantities.

With respect to cell-derived matrices, not all cell types can be efficiently isolated and expanded on any given cell-derived matrix. In fact, pluripotent stem cells (PSCs) appear to have much different requirements for a supportive growth substrate than do other types of cells. It is known that the specific cell type used to produce a matrix will have an effect on the composition of the matrix, and therefore, the reaction of various cell types to that matrix (see Marinkovic, M. et al., One size does not fit all: developing a cell-specific niche for in vitro study of cell behavior. Matrix Biol. 54-55, 426-441 (2016)). Prior work disclosed in U.S. Pat. No. 8,084,023 has described the production and composition of an extracellular matrix produced by bone marrow stromal or mesenchymal stem cells (see also Chen, X. et al., Extracellular Matrix Made by Bone Marrow Cells Facilitates Expansion of Marrow-Derived Mesenchymal Progenitor Cells and Prevents Their Differentiation into Osteoblasts. Journal of Bone and Mineral Research 22, 1943-1956 (2007) and Lai, Y. et al., Reconstitution of marrow-derived extracellular matrix ex vivo: a robust culture system for expanding large-scale highly functional human mesenchymal stem cells. Stem cells and development 19, 1095-107 (2010)). This bone marrow cell derived matrix has been shown to support the expansion of other MSCs but has not been effective for the attachment and growth of other types of stem cells, specifically, induced pluripotent stem cells (iPSCs). iPSCs have exhibited an expanded potential to form cells and tissues from a much broader category than MSCs. This represents a particularly interesting challenge, because the difficulty of growing a confluent monolayer of iPSCs in standard culture conditions makes it impractical to produce a cell-derived matrix from iPSCs. A major limitation of previous cell-derived matrices, is that in order to make a tissue-specific matrix (e.g., bone marrow matrix from bone marrow MSCs, adipose matrix from adipose MSCs, or endothelial matrix from hUVECs), it is necessary that the target population of cells already be capable of adhering to the starting substrate. A difficulty with iPSCs, embryonic stem cells(ES), and many other cell types is that they do not readily adhere to simple substrates.

### SUMMARY OF THE INVENTION

The present invention provides a solution to at least some of the aforementioned limitations and deficiencies in the art relating to cell-derived extracellular matrices (ECMs) to support the isolation, expansion and proliferation of pluripotent stem cells (PSCs), including but not limited to induced pluripotent stem cells (iPSCs) and embryonic stem cells(ES). The solution is premised on the use of an amniotic fluid cell-derived extracellular matrix. The use of uncommitted, readily adherent, and highly proliferative perinatal cells found in amniotic fluid allows for the creation of an extracellular matrix (ECM) that surprisingly, supports adhesion, isolation, expansion, and proliferation of these PSCs. This technical achievement was not possible with the cell-derived ECMs of the prior art. Additionally, the amniotic fluid cell-derived extracellular matrix (AFC-ECM) of the present invention is effective for the isolation, expansion, and proliferation of adherent cell types including but not limited to other stem cells such as mesenchymal stem cells, somatic cells, progenitor cells, mature cells, and cells from multiple germ layers. Surprisingly, the AFC-ECM of the invention supports increased proliferation of MSCs produced by bone marrow relative to a bone marrow cell-derived ECM. Furthermore, the AFC-ECM of the invention can support differentiation of stem cells into differentiated cell types.

In one aspect of the invention, disclosed is a cell-derived extracellular matrix (ECM) derived in vitro from cells isolated from amniotic fluid. In some embodiments, the ECM comprises laminin, collagen alpha-1 (XVIII), basement membrane-specific heparan sulfate proteoglycan core protein, agrin, vimentin, and collagen alpha-2 (IV), and/or isoforms thereof. In some embodiments, the isoform of collagen alpha-1 (XVIII) is isoform 2. In some embodiments, the isoform of agrin is isoform 6. In some embodiments, the cell-derived ECM further comprises fibronectin and/or an isoform thereof. In some embodiments, the cell-derived ECM does not contain any one of or all of decorin, perlecan, and collagen (III). In some embodiments, the cells isolated from amniotic fluid comprise stem cells. In some embodiments, the cell-derived ECM is decellularized. In some embodiments, the cell-derived ECM includes any one of, any combination of, or all of the components listed in Table 2 (see Example 2) and/or any variants, derivatives, or isoforms thereof listed in Table 2, preferably all of the components listed in Table 2 and/or any derivatives or isoforms thereof.

In another aspect of the invention, disclosed is method of proliferating adherent cells in culture, the method comprising culturing the adherent cells in the presence of a cell-derived extracellular matrix (ECM) in a culture media thereby proliferating the adherent cells, wherein the cell-derived ECM is derived in vitro from cells isolated from amniotic fluid. In some embodiments, the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. In some embodiments, the isoform of collagen alpha-1 (XVIII) is isoform 2. In some embodiments, the isoform of agrin is isoform 6. In some embodiments, the cell-derived ECM further comprises fibronectin and/or an isoform thereof. In some embodiments, the cell-derived ECM does not contain decorin, perlecan, or collagen (III). In some embodiments, the cells isolated from amniotic fluid comprise stem cells. In some embodiments, the cell-derived ECM is decellularized. In some embodiments, the adherent cells comprise mammalian adherent cells. In various embodiments, the adherent cells comprise stem cells, somatic cells, progenitor cells, mature cells, or cells from multiple germ layers. In some embodiments, the adherent cells comprise stem cells. In some embodiments, the stem cells are maintained in an undifferentiated state. In some embodiments, the stem cells comprise pluripotent stem cells (PSCs). In some embodiments, the PSCs comprise induced PSCs (iPSC) and/or embryonic stem cells(ES). In some embodiments, the stem cells comprise mesenchymal stem cells (MSCs). In some embodiments, the MSCs are obtained from bone marrow. In some embodiments, the adherent cells comprise progenitor cells. In some embodiments, the progenitor cells comprise endothelial progenitor cells. In some embodiments, the adherent cells comprise mature cells. In some embodiments, the mature cells comprise chondrocytes. In some embodiments, the cell-derived ECM includes any one of, any combination of, or all of the components listed in Table 2 and/or any variants, derivatives, or isoforms thereof listed in Table 2, preferably all of the components listed in Table 2 and/or any derivatives or isoforms thereof.

In another aspect of the invention, disclosed is a method of inducing differentiation of stem cells into differentiated cell types, the method comprising culturing the stem cells in the presence of a cell-derived extracellular matrix (ECM) in a differentiation media, wherein the cell-derived ECM is derived in vitro from cells isolated from amniotic fluid. In some embodiments, the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. In some embodiments, the isoform of collagen alpha-1 (XVIII) is isoform 2. In some embodiments, the isoform of agrin is isoform 6. In some embodiments, the cell-derived ECM further comprises fibronectin and/or an isoform thereof. In some embodiments, the cell-derived ECM does not contain decorin, perlecan, or collagen (III). In some embodiments, the cells isolated from amniotic fluid comprise stem cells. In some embodiments, the cell-derived ECM is decellularized. In some embodiments, the stem cells comprise pluripotent stem cells (PSCs). In some embodiments, the PSCs comprise induced PSCs (iPSC). In some embodiments, the PSCs comprise embryonic stem cells(ES). In some embodiments, the stem cells comprise mesenchymal stem cells (MSCs). In some embodiments, the MSCs are obtained from bone marrow. In some embodiments, the differentiated cell types comprise adipocytes, osteoblasts, chondrocytes, or myocytes. In some embodiments, the cell-derived ECM includes any one of, any combination of, or all of the components listed in Table 2 and/or any variants, derivatives, or isoforms thereof listed in Table 2, preferably all of the components listed in Table 2 and/or any derivatives or isoforms thereof.

In another aspect of the invention, disclosed is a method of producing a cell-derived extracellular matrix (ECM) in vitro, the method comprising:
(a) isolating cells from amniotic fluid,
(b) seeding the isolated cells onto a cell culture container or onto a cell culture container coated with a substrate,
(c) adding a culture media to the cell culture container, and
(d) culturing the cells, thereby producing a cell-derived ECM, and
(e) optionally decellularizing the cell-derived ECM.
   In some embodiments, the isolated cells from the amniotic fluid comprise stem cells. In some embodiments, the substrate is fibronectin. In some embodiments, the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. In some embodiments, the isoform of collagen alpha-1 (XVIII) is isoform 2. In some embodiments, the isoform of agrin is isoform 6. In some embodiments, the cell-derived ECM further comprises fibronectin and/or an isoform thereof. In some embodiments, the cell-derived ECM does not contain decorin, perlecan, or collagen (III). In some embodiments, the cell-derived ECM includes any one of, any combination of, or all of the components listed in Table 2 and/or any variants, derivatives, or isoforms thereof listed in Table 2.

In another aspect of the invention, disclosed is an amniotic fluid cell-derived extracellular matrix (AFC-ECM) produced in vitro by the method comprising:
(a) isolating cells from amniotic fluid,
(b) seeding the isolated cells onto a cell culture container or onto a cell culture container coated with a substrate,
(c) adding a culture media to the cell culture container, and
(d) culturing the cells, thereby producing the AFC-ECM, and
(e) optionally decellularizing the AFC-ECM.
   In some embodiments, the isolated cells from the amniotic fluid comprise stem cells. In some embodiments, the substrate is fibronectin. In some embodiments, the AFC-ECM comprises laminin, collagen alpha-1 (XVIII), basement membrane-specific heparan sulfate proteoglycan core protein, agrin, vimentin, and collagen alpha-2 (IV), and/or isoforms thereof. In some embodiments, the isoform of collagen alpha-1 (XVIII) is isoform 2. In some embodiments, the isoform of agrin is isoform 6. In some embodiments, the AFC-ECM further comprises fibronectin and/or an isoform thereof. In some embodiments, the AFC-ECM does not contain decorin, perlecan, or collagen (III). In some embodiments, the AFC-ECM includes any one of, any combination of, or all of the components listed in Table 2 and/or any variants, derivatives, or isoforms thereof listed in Table 2.

In another aspect of the invention, disclosed is a cell-derived extracellular matrix (ECM) derived in vitro from cells isolated from an umbilical cord. In some embodiments, the cells isolated from the umbilical cord are from the cord blood and/or the Wharton's jelly.

In another aspect of the invention, disclosed is a cell-derived extracellular matrix (ECM) derived in vitro from cells isolated from placenta tissue. In some embodiments, the cells isolated from the placenta tissue are from the membrane sheets (amnion and/or chorion), the villi, and/or the blood.

Also disclosed in the context of the present invention are embodiments 1 to 53. Embodiment 1 is a cell-derived extracellular matrix (ECM) derived in vitro from cells isolated from amniotic fluid. Embodiment 2 is the cell-derived ECM of embodiment 1, wherein the ECM comprises laminin, collagen alpha-1 (XVIII), basement membrane-specific heparan sulfate proteoglycan core protein, agrin, vimentin, and collagen alpha-2 (IV), and/or isoforms thereof. Embodiment 3 is the cell-derived ECM of embodiment 2, wherein the isoform of collagen alpha-1 (XVIII) is isoform 2, and/or wherein the isoform of agrin is isoform 6. Embodiment 4 is the cell derived ECM of any one of embodiments 2 or 3, wherein the cell-derived ECM further comprises fibronectin and/or an isoform thereof. Embodiment 5 is the cell-derived ECM of any one of embodiments 1 to 4, wherein the cell-derived ECM does not contain decorin, perlecan, and/or collagen (III). Embodiment 6 is the cell-derived ECM of any one of embodiments 1 to 5, wherein the cells isolated from amniotic fluid comprise stem cells. Embodiment 7 is the cell-derived ECM of any one of embodiments 1 to 6, wherein the cell-derived ECM is decellularized.

Embodiment 8 is a method of proliferating adherent cells in culture, the method comprising culturing the adherent cells in the presence of a cell-derived extracellular matrix (ECM) in a culture media thereby proliferating the adherent cells, wherein the cell-derived ECM is derived in vitro from cells isolated from amniotic fluid. Embodiment 9 is the method of embodiment 8, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. Embodiment 10 is the method of embodiment 9, wherein the isoform of collagen alpha-1 (XVIII) is isoform 2, and/or wherein the isoform of agrin is isoform 6. Embodiment 11 is the method of any one of embodiments 9 or 10, wherein the cell-derived ECM further comprises fibronectin and/or an isoform thereof. Embodiment 12 is the method of any one of embodiments 8 to 11, wherein the cell-derived ECM does not contain decorin, perlecan, and/or collagen (III). Embodiment 13 is the method of any one of embodiments 8 to 12, wherein the cells isolated from amniotic fluid comprise stem cells. Embodiment 14 is the method of any one of embodiments 8 to 13, wherein the cell-derived ECM is decellularized prior to contact with the adherent stem cells. Embodiment 15 is the method of any one of embodiments 8 to 14, wherein the adherent cells comprise mammalian adherent cells. Embodiment 16 is the method of any one of embodiments 8 to 15, wherein the adherent cells comprise stem cells, somatic cells, progenitor cells, mature cells, or cells from multiple germ layers. Embodiment 17 is the method of embodiment 16, wherein the adherent cells comprise stem cells. Embodiment 18 is the method of embodiment 17, wherein the stem cells are maintained in an undifferentiated state. Embodiment 19 is the method any one of embodiments 17 or 18, wherein the stem cells comprise pluripotent stem cells (PSCs). Embodiment 20 is the method of embodiment 19, wherein the PSCs comprise induced PSCs (iPSC). Embodiment 21 is the method of embodiment 19, wherein the PSCs comprisc embryonic stem cells(ES). Embodiment 22 is the method of any one of embodiments 17 or 18, wherein the stem cells comprise mesenchymal stem cells (MSCs). Embodiment 23 is the method of embodiment 22, wherein the MSCs are obtained from bone marrow. Embodiment 24 is the method of embodiment 16, wherein the adherent cells comprise progenitor cells. Embodiment 25 is the method of embodiment 24, wherein the progenitor cells comprise endothelial progenitor cells. Embodiment 26 is the method of embodiment 16, wherein the adherent cells comprise mature cells. Embodiment 27 is the method of embodiment 26, wherein the mature cells comprise chondrocytes.

Embodiment 28 is a method of inducing differentiation of stem cells into differentiated cell types, the method comprising culturing the stem cells in the presence of a cell-derived extracellular matrix (ECM) in a differentiation media, wherein the cell-derived ECM is derived in vitro from cells isolated from amniotic fluid. Embodiment 29 is the method of embodiment 28, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. Embodiment 30 is the method of embodiment 29, wherein the isoform of collagen alpha-1 (XVIII) is isoform 2, and/or wherein the isoform of agrin is isoform 6. Embodiment 31 is the method of any one of embodiments 29 or 30, wherein the cell-derived ECM further comprises fibronectin and/or an isoform thereof. Embodiment 32 is the method of any one of embodiments 28 to 31, wherein the cell-derived ECM does not contain decorin, perlecan, and/or collagen (III). Embodiment 33 is the method of any one of embodiments 28 to 32, wherein the cells isolated from amniotic fluid comprise stem cells. Embodiment 34 is the method of any one of embodiments 28 to 33, wherein the stem cells comprise pluripotent stem cells (PSCs). Embodiment 35 is the method of embodiment 34, wherein the PSCs comprise induced PSCs (iPSC). Embodiment 36 is the method of embodiment 34, wherein the PSCs comprise embryonic stem cells(ES). Embodiment 37 is the method of any one of embodiments 28 to 33, wherein the stem cells comprise mesenchymal stem cells (MSCs). Embodiment 38 is the method of embodiment 22, wherein the MSCs are obtained from bone marrow. Embodiment 39 is the method of any one of embodiments 37 or 38, wherein the differentiated cell types comprise adipocytes, osteoblasts, chondrocytes, or myocytes.

Embodiment 40 is a method of producing a cell-derived extracellular matrix (ECM) in vitro, the method comprising: (a) isolating cells from amniotic fluid; (b) seeding the isolated cells onto a cell culture container or onto a cell culture container coated with a substrate; (c) adding a culture media to the cell culture container; and (d) culturing the cells, thereby producing a cell-derived ECM; and (e) optionally decellularizing the cell-derived ECM. Embodiment 41 is the method of embodiment 40, wherein the isolated cells from the amniotic fluid comprise stem cells. Embodiment 42 is the method of embodiment 40 or 41, wherein the substrate is fibronectin. Embodiment 43 is the method of any one of embodiments 40 to 42, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. Embodiment 44 is the method of embodiment 43, wherein the isoform of collagen alpha-1 (XVIII) is isoform 2, and/or wherein the isoform of agrin is isoform 6. Embodiment 45 is the method of any one of embodiments 43 or 44, wherein the cell-derived ECM further comprises fibronectin and/or an isoform thereof. Embodiment 46 is the method of any one of embodiments 40 to 45, wherein the cell-derived ECM does not contain decorin, perlecan, and/or collagen (III).

Embodiment 47 is an amniotic fluid cell-derived extracellular matrix (AFC-ECM) produced in vitro by the method comprising: (a) isolating cells from amniotic fluid, (b) seeding the isolated cells onto a cell culture container or onto a cell culture container coated with a substrate, (c) adding a culture media to the cell culture container, and (d) culturing the cells, thereby producing the AFC-ECM, and (c) optionally decellularizing the AFC-ECM. Embodiment 48 is the method of embodiment 47, wherein the isolated cells from the amniotic fluid comprise stem cells. Embodiment 49 is the method of embodiment 47 or 48, wherein the substrate is fibronectin. Embodiment 50 is the method of any one of embodiments 47 to 49, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. Embodiment 51 is the method of any embodiment 50, wherein the isoform of collagen alpha-1 (XVIII) is isoform 2, and/or wherein the isoform of agrin is isoform 6. Embodiment 52 is the method of any one of embodiments 50 or 51, wherein the cell-derived ECM further comprises fibronectin and/or an isoform thereof. Embodiment 53 is the method of any one of embodiments 47 to 52, wherein the cell-derived ECM does not contain decorin, perlecan, and/or collagen (III).

The term “stem-like characteristic” or “stemness” refers a characteristic that can be observed in or associated with a stem cell. For example, one of the stem-like characteristics can be chosen from an ability of differentiating into different cell types, an ability of self-renewal, an ability of survival under certain conditions (including hypoxia conditions), an ability to resist chemotherapeutic agents, and an expression of a wide variety of markers.

The term “isoform” refers to different forms of a biomolecule or a protein. The different forms of the biomolecule or protein may be produced by a variety of processes or mechanisms. In embodiments in which the biomolecule is a protein, the isoforms may be proteins that differ in sequence by one or more amino acids. For example, the protein isoforms may be genetic alleles. Alternatively, the protein isoforms may be the products of alternate splicing, RNA editing, posttranslational processing, and the like.

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “wt. %,” “vol. %,” or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The use of the word “a” or “an” when used in conjunction with the terms “comprising,” “having,” “including,” or “containing” (or any variations of these words) may mean “one,” but it is also consistent with the meaning of “one or more,” “at least one,” and “one or more than one.”

The compositions and methods for their use can “comprise,” “consist essentially of,” or “consist of” any of the ingredients or steps disclosed throughout the specification. With respect to the transitional phrase “consisting essentially of,” in one non-limiting aspect, a basic and novel characteristic of the cell-derived extracellular matrix of the present invention is that it (1) is derived in vitro from cells isolated from amniotic fluid and (2) provides an environment that supports adhesion, isolation, expansion, and/or proliferation of PSCs.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: a photomicrograph of a Brightfield Image of the amniotic fluid cell-derived ECM at 100× power using a 10× objective lens

FIG. 2: an atomic force photomicrograph of 3 representative 40×40 um sections of the amniotic fluid cell-derived ECM and a bone marrow cell-derived ECM showing topography, adhesion, and stiffness.

FIG. 3a: a scatter plot showing quantification of adhesion of bone marrow- and amniotic fluid-cell-derived ECMs. Each point represents an independent point of measurement.

FIG. 3b: a scatter plot showing quantification of stiffness (elastic modulus) of bone marrow-a nd amniotic fluid-cell-derived ECMs. Each point represents an independent point of measurement.

FIG. 4: a photomicrograph showing Day 0 and Day 2 culture of iPSCs on amniotic fluid cell-derived ECM and a bone marrow cell-derived ECM.

FIG. 5: a plot of growth curves of iPSCs cultured in the presence of the amniotic fluid cell-derived ECM and a bone marrow cell-derived ECM.

FIG. 6: a bar graph of pluripotent transcription factor expression in iPSCs cultured on amniotic fluid cell-derived ECM.

FIG. 7: photomicrographs of Brightfield Images of MSCs, EPCs, and Chondrocytes in culture on amniotic fluid cell-derived ECM vs. bone marrow ECM vs. tissue culture.

FIG. 8a: a bar graph quantifying the proliferation of MSCs in culture after 4 days on amniotic fluid cell-derived ECM.

FIG. 8b: a bar graph quantifying the proliferation of EPCs in culture after 4 days on amniotic fluid cell-derived ECM.

FIG. 8c: a bar graph quantifying the proliferation of chondrocytes in culture after 4 days on amniotic fluid cell-derived ECM.

FIG. 9: a bar graph of the percentage of MSCs expressing SSEA-4 as measured by flow cytometry following culture on amniotic fluid cell-derived ECM vs. bone marrow cell ECM vs. tissue culture plastic for four days

FIG. 10: a photomicrograph of adipogenic differentiation of MSCs cultured on amniotic fluid cell-derived matrix for four days.

FIG. 11: a photomicrograph of osteogenic differentiation of MSCs cultured on amniotic fluid cell-derived matrix for four days.

### DETAILED DESCRIPTION OF THE INVENTION

The present invention discloses a cell-derived extracellular matrix (ECM) derived in vitro from cells isolated from amniotic fluid and methods of making the ECM. Also disclosed are methods of using the ECM for the isolation, maintenance, and expansion/proliferation of adherent cells which can be mammalian cells.

The function of mammalian cells is determined, largely, by the environment, e.g., an extracellular matrix, in which they reside. They react to signals that are present in their environment (positive signals) and also to signals that are required but are not present (negative signals). It is likely that uncommitted stem cells can produce a matrix that contains niche motifs necessary to maintain stem cell viability and stemness, but lack many lineage specific signals that more mature cells may secrete which would push a stem cell toward a particular fate. Without being bound by theory, it is suggested that a less mature cell, e.g., a perinatal cell or perinatal stem cell, may produce an ECM that is different from ECMs disclosed previously in the art, such as bone marrow stromal cell-derived ECMs, and may allow for better isolation and expansion/proliferation of stem cells with higher potential than mesenchymal stem cells (MSCs), such as pluripotent stem cells (PSCs). Mass spectrometry demonstrated, that compared to previously known cell-derived ECMs, the amniotic fluid cell-derived ECM of the invention contains matrix proteins found in all 3 germ layers and lacked specific proteins strongly associated with osteogenic lineages. Moreover, the ECM of the invention contained specific motifs, such as laminin, that are known to facilitate pluripotent cell adhesion and expansion.

### A. Amniotic Fluid Cell-Derived Extracellular Matrix (AFC-ECM)

Perinatal cells can be divided into three groups: cells from amniotic fluid; cells from the placenta; and cells from the umbilical cord. Amniotic fluid has several sources of cells including cells derived from the developing fetus sloughed from the fetal amnion membrane, skin, and alimentary, respiratory, and urogenital tracts. Placenta also has several sources of cells including the membrane sheets (amnion and chorion), the villi, and the blood. Umbilical cord cells generally come from two sources, cord blood and Wharton's jelly. The cells from these three perinatal sources can include stem cells. The cells used to produce the amniotic fluid cell-derived ECM of the invention are obtained from the amniotic fluid of a mammal including but not limited to a human (homo sapiens), murine, rabbit, cat, dog, pig, equine, or primate. In preferred embodiments, the cells are from the amniotic fluid of a human. The amniotic fluid can be sourced from humans at full-term births (greater than about 37 weeks gestational age) or pre-term births (less than about 37 weeks gestational age). Pre-term births include late pre-term births (about 33 to about 37 weeks gestational age) moderate pre-term births (about 29 to about 33 weeks gestational age), and extreme pre-term births (about 23 to about 29 weeks gestational age). The amniotic fluid can be sourced from humans prior to birth at any gestational age where amniotic fluid is present, and can be combined with sources of amniotic fluid from births. Generally, prior to birth, amniotic fluid is collected by an amniocentesis procedure. In some embodiments the amniotic fluid is sourced from humans at full-term births, at pre-term births, at late pre-term births, at moderate pre-term births, at extreme pre-term births, or prior to birth, or combinations thereof. In some embodiments, the amniotic fluid is sourced prior to birth and is collected from about 10 weeks gestational age up to birth, or from about 10 weeks to about 23 weeks gestational age, or from about 10 weeks to about 16 weeks gestational age, or from about 12 weeks gestational age up to birth, or from about 12 weeks to about 23 weeks gestational age, or from about 12 weeks to about 16 weeks gestational age. In some embodiments, the amniotic fluid sourced prior to birth is collected by an amniocentesis procedure. The cells can be obtained and isolated from amniotic fluid by techniques known in the art, such as those disclosed in Murphy et. al., Amniotic Fluid Stem Cells, Perinatal Stem Cells, Second Ed. 2013.

Amniotic fluid is comprised of cells having the ability to differentiate into cell types derived from all 3 embryonic germ layers (ectoderm, endoderm, mesoderm) spontaneously or as a result of treatment with specific growth factors or combinations of growth factors known to one of skill in the art. That is, a single cell has the capacity to be induced to express genes which are specific to any of the three germ layers. Amniotic fluid also contains a mixture of different cell types including cells derived from the developing fetus sloughed from the fetal amnion membrane, skin, and alimentary, respiratory, and urogenital tracts. Because of the origin of the amniotic fluid and placental membranes, these cells can maintain highly multipotent differentiation potential and comprise a cell population that contains cells of all three germ layers. The amniotic fluid cells can comprise stem cells. In some embodiments, the amniotic fluid cells are isolated stem cells. In some embodiments, the amniotic fluid cells comprise stem cells having the ability to differentiate into cell types derived from all 3 embryonic germ layers (ectoderm, endoderm, mesoderm) and/or multipotent stem cells, and/or pluripotent stem cells.

The amniotic fluid cell-derived ECM of the invention is comprised of various proteins. The proteins of the ECM can be identified by techniques known in the art and include mass spectroscopy and immunohistochemical staining. The ECM can include, but is not limited to the components listed in Table 2 (see Example 2 below) and any variants, derivatives, or isoforms thereof. The amniotic fluid-cell derived ECM can include any combination of any of the components and any variants, derivatives, or isoforms thereof from Table 2. In some embodiments, a combination can comprise, consist essentially of, or consist of: laminin, collagen alpha-1 (XVIII), basement membrane-specific heparan sulfate proteoglycan core protein, agrin, vimentin, and collagen alpha-2 (IV), and/or isoforms thereof. In some embodiments, the isoform of collagen alpha-1 (XVIII) is isoform 2. In some embodiments, the isoform of agrin is isoform 6. In some embodiments, the cell-derived ECM further comprises, consist essentially of, or consists of fibronectin and/or an isoform thereof. In some embodiments, the amniotic fluid cell-derived ECM does not contain any one of or all of decorin, perlecan, and collagen (III). The most abundant collagens in Table 2 are collagens I, IV, and XVIII. Some noteworthy differences in proteins between the amniotic fluid cell-derived ECM of the present inventions and a bone marrow cell-derived matrix are described in Table 1.

TABLE 1
Differences Between the Amniotic Fluid Cell-Derived ECM (AFC-
Matrix) and a Bone Marrow Cell-Derived Matrix (BM-Matrix)

| Protein/ | | |
|---|---|---|
| Gene | Difference between AFC- | |
| Code | Matrix & BM-Matrix | Physiologic Relevance |
| Laminin | 5 sub-units are abundant | Laminin is known to |
| | in AFC Matrix; Low | support adhesion and |
| | expression in BM-Matrix | expansion of pluripotent |
| | | cells. |
| Collagen | Abundant in AFC Matrix; | Important for ocular |
| XVIII | Absent in BM-Matrix | development |
| Agrin | Present in AFC Matrix; | produced by motoneurons |
| | Absent in BM-Matrix | to induce aggregation of |
| | | acetylcholine receptors |
| Biglycan | Abundant in BM-Matrix; | Regulates bone and muscle |
| | Low expression in AFC Matrix | development |
| Collagen I | Overexpressed in BM-Matrix | Key to fibrillar proteins; |
| | relative to AFC Matrix | highly abundant in bone |
| Periostin | Present in BM-Matrix; | Regulates mineralization; |
| | Absent in AFC Matrix | Marker of |
| | | noncardiomyocyte lineage |
| | | cells in heart |

The amniotic fluid cell-derived ECM (AFC-ECM) of the invention can be produced in vitro by the following process:
(a) isolating cells from amniotic fluid,
(b) seeding the isolated cells onto a cell culture container or onto a cell culture container coated with a substrate,
(c) adding a culture media to the cell culture container, and
(d) culturing the cells, thereby producing the AFC-ECM, and
(e) optionally decellularizing the AFC-ECM.

Disclosed herein is a method of producing in vitro a cell-derived extracellular matrix (ECM), the method comprising:
(a) isolating cells from amniotic fluid,
(b) seeding the isolated cells onto a cell culture container or onto a cell culture container coated with a substrate,
(c) adding a culture media to the cell culture container, and
(d) culturing the cells, thereby producing a cell-derived ECM, and
(e) optionally decellularizing the cell-derived ECM.

Disclosed herein is an amniotic fluid cell-derived extracellular matrix (AFC-ECM) produced in vitro by the method comprising:
(a) isolating cells from amniotic fluid,
(b) seeding the isolated cells onto a cell culture container or onto a cell culture container coated with a substrate,
(c) adding a culture media to the cell culture container, and
(d) culturing the cells, thereby producing the AFC-ECM, and
(e) optionally decellularizing the AFC-ECM.

Any seeding density may be used which allows cells to form a confluent monolayer immediately or after a period of time in culture. In some embodiments, the seeding density is about 10 cells/cm²-about 100,000 cells/cm², or about 100 cells/cm²-about 75,000 cells/cm², or about 500 cells/cm²-about 50,000 cells/cm², or about 500 cells/cm²-about 10,000 cells/cm², or about 500 cells/cm²-about 5,000 cells/cm², or about 500 cells/cm²-about 2,500 cells/cm², or about 1,000 cells/cm²-about 25,000 cells/cm², or about 2,000 cells/cm²-about 10,000 cells/cm², or about 3,000 cells/cm²-about 5000 cells/cm².

Any type of container suitable for cultivation of cells can be used for the present invention. Examples include, but are not limited to cell culture flasks, T-flasks, stirred flasks, spinner flasks, fermenters, and bioreactors. Rocking bottles, shaking flasks, tubes, and other containers are also suitable containers when placed on a rocking platform or shaker. The cell culture container can be coated with a substrate to allow for better cell adhesion. A non-limiting example of a suitable substrate for coating the cell container is fibronectin.

Various commercially available cell culture media, e.g., alpha Minimum Essential Media (α-MEM) culture media (Thermo Fisher Scientific, Grand Island, NY), are suitable for culturing amniotic fluid cells. The commercially available culture media can be modified by adding various supplemental substances to the media, e.g. sodium bicarbonate, L-glutamine, penicillin, streptomycin, Amphotericin B and/or serum. The serum can be fetal bovine serum. The media can also be serum free. Additionally, substances such as L-ascorbic acid can be added to the media or modified media to induce cell production of an ECM.

The initial culture media can be changed and/or replaced with another media at various times during the culturing process. For example, the initial media can be a “Complete Media” and then be replaced by an “Inducing Media” during the culturing process. A non-limiting example of a “Complete Media” contains (α-MEM) plus 2 mM L-Glutamine plus antibiotic-antimycotic plus 15% Fetal Bovine Serum. A non-limiting example of an “Inducing Media” contains the “Complete Media” plus 50 mM L-Ascorbic Acid.

The culturing of the amniotic fluid cells can take place in an incubator at 37° C., 5% CO₂, and 90% humidity. Culturing can take place under various environmental conditions including, but not limited to normoxic, i.e., 20-21% oxygen in the atmosphere, or hypoxic conditions.

Decellularizing the amniotic fluid cell-derived ECM of the amniotic fluid cells can include removing the viable amniotic fluid cells or rendering the amniotic fluid cells non-viable. The amniotic fluid cells can be decellularized from the ECM by using methods known in the art and can include, but are not limited to lysing the amniotic fluid cells and then removing the lysed amniotic fluid cells by washing. Various substances can be used to remove the amniotic fluid cells from the ECM. Non-limiting examples include an “Extraction Buffer” containing TRITON X-100 and ammonium hydroxide in PBS buffer. After the ECM has been decellularized of amniotic fluid cells, the resulting ECM is thereby essentially cell-free or free of viable amniotic fluid cells. If feeder cells are used, then the decellularizing methods also apply to any viable feeder cells present on the ECM, thereby resulting in the ECM being essentially free or free of viable feeder cells. The decellularizing methods also apply to any viable cells present on the ECM, thereby resulting in the ECM being essentially free or free of any viable cells. Thus, a decellularized ECM means that the ECM is acellular, meaning that the ECM is free of any viable cells.

In some embodiments, the amniotic fluid cell-derived ECM is a three-dimensional (3D) ECM.

The methods described supra also apply to producing cell-derived ECMs from other perinatal cells such as cells from the umbilical cord including the cord blood and Wharton's jelly; and cells from placenta tissue including the membrane sheets (amnion and chorion), the villi and the blood.

In one embodiment, a perinatal cell-derived ECM is produced in vitro by the following process:
(a) isolating cells from an umbilical cord,
(b) seeding the isolated cells onto a cell culture container or onto a cell culture container coated with a substrate,
(c) adding a culture media to the cell culture container, and
(d) culturing the cells, thereby producing a cell-derived ECM, and
(e) optionally decellularizing the cell-derived ECM.
   In some embodiments, the cells isolated from the umbilical cord are from the cord blood and/or the Wharton's jelly.

In another embodiment, a perinatal cell-derived ECM is produced in vitro by the following process:
(a) isolating cells from placenta tissue,
(b) seeding the isolated cells onto a cell culture container or onto a cell culture container coated with a substrate,
(c) adding a culture media to the cell culture container, and
(d) culturing the cells, thereby producing a cell-derived ECM, and
(e) optionally decellularizing the cell-derived ECM.
   In some embodiments, the cells isolated from the placenta tissue are from the membrane sheets (amnion and/or chorion), the villi, and/or the blood.

In one aspect of the invention, disclosed is a cell-derived extracellular matrix (ECM) derived in vitro from cells isolated from an umbilical cord. In some embodiments, the cells isolated from the umbilical cord are from the cord blood and/or the Wharton's jelly.

In another aspect of the invention, disclosed is a cell-derived extracellular matrix (ECM) derived in vitro from cells isolated from placenta tissue. In some embodiments, the cells isolated from the placenta tissue are from the membrane sheets (amnion and/or chorion), the villi, and/or the blood

### B. Methods to Expand/Proliferate Adherent Cells

Methods to expand/proliferate adherent cells include obtaining the adherent cells and culturing them in the presence of the amniotic fluid cell-derived ECM of the invention. In some embodiments, the adherent cells are mammalian adherent cells. Any seeding density may be used which allows cells to form a confluent monolayer immediately or after a period of time in culture. In some embodiments, the seeding density is about 10 cells/cm²-about 100,000 cells/cm², or about 100 cells/cm²-about 75,000 cells/cm², or about 500 cells/cm²-about 50,000 cells/cm², or about 500 cells/cm²-about 10,000 cells/cm², or about 500 cells/cm²-about 5,000 cells/cm², or about 500 cells/cm²-about 2,500 cells/cm², or about 1,000 cells/cm²-about 25,000 cells/cm², or about 2,000 cells/cm²-about 10,000 cells/cm², or about 3,000 cells/cm²-about 5000 cells/cm². In various embodiments, the adherent cells are stem cells, somatic cells, progenitor cells, mature cells, and/or cells from multiple germ layers.

In some embodiments, the adherent cells are stem cells. In some embodiments, the stem cells are pluripotent stem cells (PSCs) or mesenchymal stem cells (MSCs). Pluripotent stem cells (PSCs) can self-renew and differentiate into any of the three germ layers: ectoderm, endoderm, and mesoderm, from which all tissues and organs develop. Embryonic stem cells(ES) are currently the only known natural pluripotent stem cells. Induced pluripotent stem (iPSCs) cells also are PSCs. iPSCs are derived from cells generally taken from adult tissue or adult cells, and reprogrammed to the level of embryonic stem cells. Methods for producing iPSCs are known in the art. Mesenchymal stem cells (MSCs) are multipotent stromal cells. MSCs can be obtained from the following non-limiting sources: bone marrow, umbilical cord tissue, e.g. Wharton's jelly, umbilical cord blood, adipose tissue, and amniotic fluid. In some embodiments the stem cells are maintained in an undifferentiated state and maintain their stemness.

In some embodiments, the adherent cells are somatic cells also known as vegetal cells, which are any biological cells in a multicellular organism other than reproductive cells, gametes, germ cells, gametocytes, or undifferentiated stem cells.

In some embodiments, the adherent cells are cells from multiple germ layers including cells from the ectoderm, endoderm, and/or mesoderm.

In some embodiments, the adherent cells are progenitor cells including but not limited to endothelial progenitor cells (EPCs), angioblasts, pancreatic progenitor cells, progenitor cells from the periosteum, and bone marrow stromal cells.

In some embodiments, the adherent cells are mature cells including but not

limited to chondrocytes and osteoblasts.

Cell culture techniques suitable for proliferation of adherent cells in culture are known in the art and can be followed. Suitable commercially available culture media for cell proliferation includes, but is not limited to: StemMACS™ iPS-Brew XF available from Miltenyl Biotec; alpha minimum essential media (aMEM) which can be supplemented with fetal bovine serum, antibiotic-antimycotic (anti-anti), and/or GlutaMax™ available from Gibco; and Iscove's Modified Dulbecco's Medium (IMDM) which can be supplemented with fetal bovine serum, antibiotic-antimycotic (anti-anti), GlutaMax and/or growth factors such as EGF and FGF. In some embodiments, no Rock inhibitor is used. Once cells begin to approach confluence (e.g., as determined by brightfield microscopy), cells can be passaged manually, by cutting large colonies into smaller colonies and then re-plate those by physically lifting them off the dish and placing them on a fresh plate of amniotic fluid cell-derived ECM. This procedure can be repeated indefinitely. In some embodiments disclosed is a method of proliferating adherent cells in culture, the method comprising culturing the cells in the presence of a cell-derived extracellular matrix (ECM) in a culture media thereby proliferating the adherent cells, wherein the cell-derived ECM is derived in vitro from cells isolated from amniotic fluid.

The methods of expanding/proliferating adherent cells described supra also apply to the use of expanding/proliferating adherent cells in culture in the presence of other perinatal cell-derived ECMs. In some aspects, disclosed is a method of proliferating adherent cells in culture, the method comprising culturing the adherent cells in the presence of a cell-derived extracellular matrix (ECM) in a culture media thereby proliferating the adherent cells, wherein the cell-derived ECM is derived in vitro from cells isolated from an umbilical cord or placenta tissue. In some embodiments, the cells isolated from the umbilical cord are from the cord blood and/or the Wharton's jelly. In other embodiments, the cells isolated from the placenta tissue are from the membrane sheets (amnion and/or chorion), the villi, and/or the blood.

### C. Methods to Induce Differentiation of Stem Cells

Disclosed herein are methods of inducing differentiation of stem cells into differentiated cell types, the method comprising culturing the stem cells in the presence of a cell-derived extracellular matrix (ECM) in a differentiation media, wherein the cell-derived ECM is derived in vitro from cells isolated from amniotic fluid, i.e. the amniotic fluid cell-derived ECM of the invention. In some embodiments, the cell-derived ECM comprises laminin, collagen alpha-1 (XVIII), basement membrane-specific heparan sulfate proteoglycan core protein, agrin, vimentin, and collagen alpha-2 (IV), and/or isoforms thereof. In some embodiments, the isoform of collagen alpha-1 (XVIII) is isoform 2. In some embodiments, the isoform of agrin is isoform 6. In some embodiments, the cell-derived ECM further comprises fibronectin and/or an isoform thereof. In some embodiments, the cell-derived ECM does not contain decorin, perlecan, or collagen (III). In some embodiments, the cells isolated from amniotic fluid comprise stem cells. In some embodiments, the cell-derived ECM is decellularized. The stem cells can be pluripotent stem cells (PSCs), such as induced PSCs (iPSC) or embryonic stem cells(ES). The stem cells can be mesenchymal stem cells (MSCs). MSCs can be obtained from the following non-limiting sources: bone marrow, umbilical cord tissue, c.g. Wharton's jelly, umbilical cord blood, adipose tissue, and amniotic fluid. In some embodiments, the MSCs are obtained from bone marrow. In some embodiments, the differentiated cell types can be adipocytes, osteoblasts, chondrocytes, or myocytes.

Cell culture techniques for differentiation of stem cells into differentiated cell types are known in the art and can be followed. Various commercially available differentiation media are suitable for use and can be specific for a particular desired differentiated cell type. For example, an adipogenic differentiation media can comprise DMEM containing FBS, IBMX, dexamethasone, insulin, and/or indomethacin. Another example is an osteoblast differentiation media which can be a growth media supplemented with dexamethasone and/or L-ascorbate-2-phosphate.

The methods of inducing differentiation of stem cells described supra also apply to the use of culturing stem cells in the presence of other perinatal cell-derived ECMs in differentiation media. In some aspects, disclosed is a method of inducing differentiation of stem cells into differentiated cell types, the method comprising culturing the stem cells in the presence of a cell-derived extracellular matrix (ECM) in a differentiation media, wherein the cell-derived ECM is derived in vitro from cells isolated from an umbilical cord or placenta tissue. In some embodiments, the cells isolated from the umbilical cord are from the cord blood and/or the Wharton's jelly. In other embodiments, the cells isolated from the placenta tissue are from the membrane sheets (amnion and/or chorion), the villi, and/or the blood.

### EXAMPLES

The following examples are included to demonstrate certain non-limiting aspects of the invention. It should be appreciated by those of skill in the art that the techniques disclosed in the examples that follow represent techniques discovered by the applicants to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1—Production of an Amniotic Fluid Cell-Derived ECM

Four amniotic fluid cell-derived ECMs (Matrix A, Matrix B, Matrix C, and Matrix D) were made using the following procedure: cells aseptically isolated from amniotic fluid collected from full term birth (>37 weeks gestational age) from 4 donors were seeded onto fibronectin coated tissue-culture treated flasks and cultured in Complete Media at 37° C., 5% CO₂ and 90% RH in an incubator. The Complete Media was alpha Minimum Essential Media (aMEM) plus 2 mM L-Glutamine plus antibiotic-antimycotic plus 15% Fetal Bovine Serum.

At day 3-4, one-half of the complete medium was aspirated from the flasks and replaced with one-half of new Complete Media. The flasks were placed back into the incubator at the same conditions as stated above.

At day 7-8, the Complete Media was aspirated from the culture flasks and was replenished with Inducing Media. The flasks were placed back into the incubator at the same conditions as stated above. The Inducing Media was Complete Media plus 50 mM L-Ascorbic Acid.

At day 10-11, the Inducing Media was aspirated from the culture flasks and the ECM which had formed inside the flasks was washed one time with phosphate buffered saline (PBS). Then the PBS was aspirated from the flasks. An Extraction Buffer was added to the flasks and incubated for 7-10 minutes at RT to decellularize each ECM, then the Extraction Buffer was aspirated from the flasks. The Extraction Buffer was PBS containing 0.5% (v/v) TRITON-X100 and 20 mM ammonium hydroxide (NH₄OH).

Each of the decellularized ECMs in the flasks was washed three times with PBS followed by one wash with sterile water and then the sterile water was aspirated from the flasks. The four decellularized ECMs in the flasks were allowed to dry at RT and then stored at 4° C.

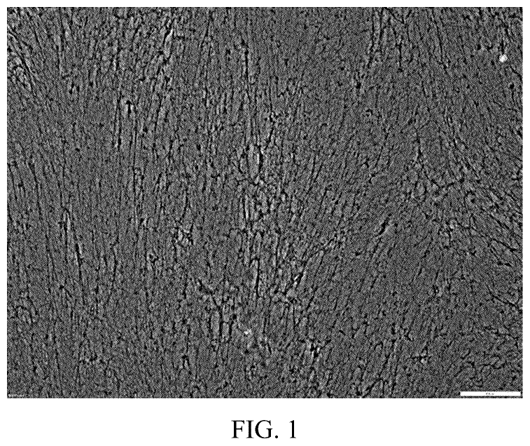

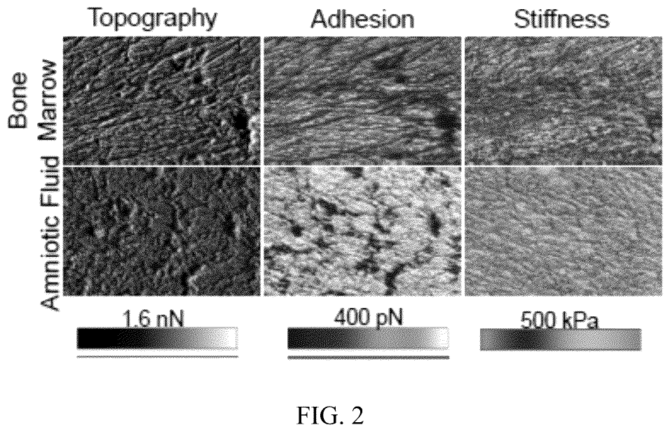

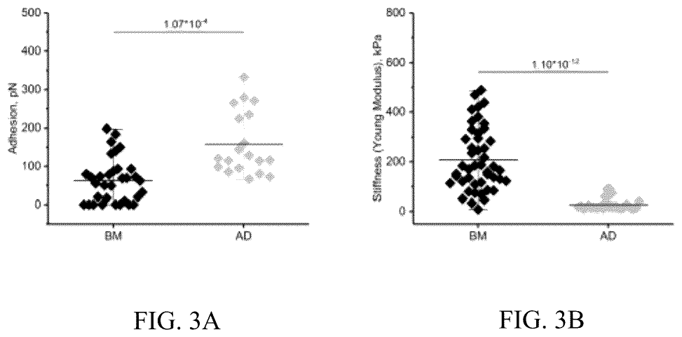

A photomicrograph of a Brightfield Image of an amniotic fluid cell-derived ECM (Matrix B) is shown in FIG. 1 at 100× power using a 10× objective lens. An atomic force photomicrograph of 3 representative 40×40 um sections of the amniotic fluid cell-derived ECM (Matrix B) and a bone marrow cell-derived ECM showing topography, adhesion, and stiffness is shown in FIG. 2. The bone marrow- and amniotic fluid-cell-derived ECMs are structurally and physically distinct. Quantification of adhesion and stiffness (elastic modulus) of bone marrow- and amniotic fluid-cell-derived ECMs show bone marrow ECM is 10-fold stiffer, and 3-fold less adhesive, relative to amniotic fluid ECM as shown in the scatter plots in FIG. 3a (Adhesion) and FIG. 3b (Stiffness) where BM=bone marrow cell-derive ECM, AD=amniotic fluid cell-derived ECM (Matrix B). Each point represents an independent point of measurement.

### Example 2—Composition of Amniotic Fluid Cell-Derived ECM

The composition of the each of the amniotic fluid cell-derived ECMs produced in Example 1 was determined by mass spectrometry. The components with their spectral count and molecular weight are listed in Table 2.

TABLE 2
Amniotic Fluid Cell-Derived ECM Components

| | | | Total Spectra Count | | | |
|---|---|---|---|---|---|---|
| Protein | MW | | Matrix A | Matrix B | Matrix C | Matrix D |
| Isoform 7 of Fibronectin OS = Homo sapiens OX = 9606 | 269 | kDa | 817 | 13965 | 1143 | 794 |
| GN = FN1 | | | | | | |
| Isoform 3 of Fibronectin OS = Homo sapiens OX = 9606 | 259 | kDa | 807 | 13836 | 1150 | 790 |
| GN = FN1 | | | | | | |
| Fibronectin OS = Homo sapiens OX = 9606 GN = FN1 PE = 1 | 263 | kDa | 802 | 13851 | 1138 | 781 |
| SV = 4 | | | | | | |
| Isoform 14 of Fibronectin OS = Homo sapiens OX = 9606 | 249 | kDa | 770 | 12625 | 1103 | 763 |
| GN = FN1 | | | | | | |
| Isoform 10 of Fibronectin OS = Homo sapiens OX = 9606 | 240 | kDa | 758 | 12506 | 1082 | 746 |
| GN = FN1 | | | | | | |
| Myosin-9 OS = Homo sapiens OX = 9606 GN = MYH9 | 227 | kDa | 564 | 3923 | 295 | 340 |
| PE = 1 SV = 4 | | | | | | |
| SWISS-PROT: P60712 (Bos taurus) Actin, cytoplasmic 1 | 42 | kDa | 293 | 2496 | 450 | 395 |
| Vimentin OS = Homo sapiens OX = 9606 GN = VIM PE = 1 | 54 | kDa | 263 | 1179 | 178 | 338 |
| SV = 4 | | | | | | |
| Neuroblast differentiation-associated protein AHNAK | 629 | kDa | 250 | 803 | 17 | 184 |
| OS = Homo sapiens OX = 9606 GN = AHNAK PE = 1 SV = 2 | | | | | | |
| Histone H2B type 1-D OS = Homo sapiens OX = 9606 | 14 | kDa | 212 | 1121 | 176 | 187 |
| GN = HIST1H2BD PE = 1 SV = 2 | | | | | | |
| Isoform 2 of Filamin-A OS = Homo sapiens OX = 9606 | 280 | kDa | 212 | 342 | 12 | 143 |
| GN = FLNA | | | | | | |
| Basement membrane-specific heparan sulfate | 469 | kDa | 202 | 0 | 402 | 338 |
| proteoglycan core protein OS = Homo sapiens OX = 9606 | | | | | | |
| GN = HSPG2 PE = 1 SV = 4 | | | | | | |
| Isoform 4 of Plectin OS = Homo sapiens OX = 9606 | 516 | kDa | 185 | 337 | 10 | 83 |
| GN = PLEC | | | | | | |
| SWISS-PROT: P02769 (Bos taurus) Bovine serum | 69 | kDa | 122 | 112 | 53 | 72 |
| albumin precursor | | | | | | |
| Tubulin beta chain OS = Homo sapiens OX = 9606 | 50 | kDa | 117 | 0 | 36 | 92 |
| GN = TUBB PE = 1 SV = 2 | | | | | | |
| Spectrin alpha chain, non-erythrocytic 1 OS = Homo | 285 | kDa | 107 | 342 | 10 | 78 |
| sapiens OX = 9606 GN = SPTAN1 PE = 1 SV = 3 | | | | | | |
| Tubulin beta-4B chain OS = Homo sapiens OX = 9606 | 50 | kDa | 107 | 0 | 34 | 84 |
| GN = TUBB4B PE = 1 SV = 1 | | | | | | |
| Isoform 3 of Spectrin alpha chain, non-erythrocytic 1 | 282 | kDa | 106 | 345 | 0 | 77 |
| OS = Homo sapiens GN = SPTAN1 | | | | | | |
| Histone H4 OS = Homo sapiens OX = 9606 | 11 | kDa | 100 | 1257 | 91 | 96 |
| GN = HIST1H4A PE = 1 SV = 2 | | | | | | |
| Tubulin beta-4A chain OS = Homo sapiens OX = 9606 | 50 | kDa | 99 | 0 | 31 | 76 |
| GN = TUBB4A PE = 1 SV = 2 | | | | | | |
| Protein-glutamine gamma-glutamyltransferase 2 | 77 | kDa | 94 | 188 | 73 | 25 |
| OS = Homo sapiens OX = 9606 GN = TGM2 PE = 1 SV = 2 | | | | | | |
| SWISS-PROT: P00761\|TRYP_PIG Trypsin - Sus scrofa | 24 | kDa | 94 | 362 | 121 | 96 |
| (Pig). | | | | | | |
| Tubulin alpha-1B chain OS = Homo sapiens OX = 9606 | 50 | kDa | 93 | 295 | 37 | 86 |
| GN = TUBA1B PE = 1 SV = 1 | | | | | | |
| Isoform 2 of Clathrin heavy chain 1 OS = Homo sapiens | 188 | kDa | 92 | 97 | 1 | 15 |
| OX = 9606 GN = CLTC | | | | | | |
| Tubulin beta-2A chain OS = Homo sapiens OX = 9606 | 50 | kDa | 92 | 0 | 32 | 73 |
| GN = TUBB2A PE = 1 SV = 1 | | | | | | |
| Elongation factor 1-alpha 1 OS = Homo sapiens OX = 9606 | 50 | kDa | 89 | 132 | 14 | 70 |
| GN = EEF1A1 PE = 1 SV = 1 | | | | | | |
| Isoform 2 of Tubulin alpha-1A chain OS = Homo sapiens | 46 | kDa | 88 | 305 | 34 | 82 |
| OX = 9606 GN = TUBA1A | | | | | | |
| Talin-1 OS = Homo sapiens OX = 9606 GN = TLN1 PE = 1 | 270 | kDa | 88 | 109 | 4 | 41 |
| SV = 3 | | | | | | |
| Myosin-10 OS = Homo sapiens GN = MYH10 PE = 1 SV = 3 | 229 | kDa | 84 | 417 | 56 | 46 |
| Spectrin beta chain, non-erythrocytic 1 OS = Homo | 275 | kDa | 84 | 260 | 7 | 61 |
| sapiens OX = 9606 GN = SPTBN1 PE = 1 SV = 2 | | | | | | |
| Pyruvate kinase PKM OS = Homo sapiens OX = 9606 | 58 | kDa | 83 | 73 | 21 | 46 |
| GN = PKM PE = 1 SV = 4 | | | | | | |
| Tubulin alpha-1C chain OS = Homo sapiens OX = 9606 | 50 | kDa | 83 | 0 | 0 | 78 |
| GN = TUBA1C PE = 1 SV = 1 | | | | | | |
| Major vault protein OS = Homo sapiens OX = 9606 | 99 | kDa | 82 | 360 | 68 | 108 |
| GN = MVP PE = 1 SV = 4 | | | | | | |
| Actin, aortic smooth muscle OS = Homo sapiens | 42 | kDa | 78 | 0 | 126 | 77 |
| OX = 9606 GN = ACTA2 PE = 1 SV = 1 | | | | | | |
| Actin, gamma-enteric smooth muscle OS = Homo sapiens | 42 | kDa | 76 | 313 | 122 | 73 |
| OX = 9606 GN = ACTG2 PE = 1 SV = 1 | | | | | | |
| Alpha-actinin-4 OS = Homo sapiens OX = 9606 | 105 | kDa | 75 | 199 | 11 | 76 |
| GN = ACTN4 PE = 1 SV = 2 | | | | | | |
| Isoform 8 of Filamin-B OS = Homo sapiens OX = 9606 | 282 | kDa | 75 | 123 | 2 | 94 |
| GN = FLNB | | | | | | |
| Tubulin alpha-4A chain OS = Homo sapiens OX = 9606 | 50 | kDa | 73 | 262 | 0 | 65 |
| GN = TUBA4A PE = 1 SV = 1 | | | | | | |
| Glyceraldehyde-3-phosphate dehydrogenase OS = Homo | 36 | kDa | 71 | 145 | 24 | 66 |
| sapiens OX = 9606 GN = GAPDH PE = 1 SV = 3 | | | | | | |
| Cytoplasmic dynein 1 heavy chain 1 OS = Homo sapiens | 532 | kDa | 65 | 49 | 0 | 5 |
| OX = 9606 GN = DYNC1H1 PE = 1 SV = 5 | | | | | | |
| Histone H2A.J OS = Homo sapiens OX = 9606 GN = H2AFJ | 14 | kDa | 65 | 216 | 62 | 79 |
| PE = 1 SV = 1 | | | | | | |
| Serpin H1 OS = Homo sapiens OX = 9606 GN = SERPINH1 | 46 | kDa | 64 | 731 | 128 | 97 |
| PE = 1 SV = 2 | | | | | | |
| Histone H2A type 2-C OS = Homo sapiens OX = 9606 | 14 | kDa | 63 | 0 | 61 | 82 |
| GN = HIST2H2AC PE = 1 SV = 4 | | | | | | |
| Alpha-actinin-1 OS = Homo sapiens GN = ACTN1 PE = 1 | 103 | kDa | 62 | 217 | 19 | 77 |
| SV = 2 | | | | | | |
| Histone H2AX OS = Homo sapiens OX = 9606 | 15 | kDa | 61 | 0 | 69 | 75 |
| GN = H2AFX PE = 1 SV = 2 | | | | | | |
| Tubulin beta-3 chain OS = Homo sapiens OX = 9606 | 50 | kDa | 58 | 194 | 28 | 53 |
| GN = TUBB3 PE = 1 SV = 2 | | | | | | |
| Endoplasmic reticulum chaperone BiP OS = Homo | 72 | kDa | 57 | 0 | 35 | 46 |
| sapiens OX = 9606 GN = HSPA5 PE = 1 SV = 2 | | | | | | |
| Myosin regulatory light chain 12A OS = Homo sapiens | 20 | kDa | 57 | 0 | 244 | 153 |
| OX = 9606 GN = MYL12A PE = 1 SV = 1 | | | | | | |
| Myosin regulatory light chain 12B OS = Homo sapiens | 20 | kDa | 57 | 0 | 240 | 149 |
| OX = 9606 GN = MYL12B PE = 1 SV = 2 | | | | | | |
| Elongation factor 2 OS = Homo sapiens OX = 9606 | 95 | kDa | 53 | 72 | 3 | 45 |
| GN = EEF2 PE = 1 SV = 4 | | | | | | |
| Filamin-C OS = Homo sapiens OX = 9606 GN = FLNC | 291 | kDa | 53 | 62 | 3 | 63 |
| PE = 1 SV = 3 | | | | | | |
| Histone H3.1 OS = Homo sapiens OX = 9606 | 15 | kDa | 53 | 0 | 24 | 33 |
| GN = HIST1H3A PE = 1 SV = 2 | | | | | | |
| 60 kDa heat shock protein, mitochondrial OS = Homo | 61 | kDa | 52 | 58 | 14 | 58 |
| sapiens OX = 9606 GN = HSPD1 PE = 1 SV = 2 | | | | | | |
| Tubulin beta-6 chain OS = Homo sapiens OX = 9606 | 50 | kDa | 52 | 0 | 23 | 52 |
| GN = TUBB6 PE = 1 SV = 1 | | | | | | |
| Isoform 4 of Collagen alpha-1(XII) chain OS = Homo | 325 | kDa | 51 | 35 | 36 | 27 |
| sapiens OX = 9606 GN = COL12A1 | | | | | | |
| Nucleophosmin OS = Homo sapiens OX = 9606 | 33 | kDa | 48 | 103 | 15 | 43 |
| GN = NPM1 PE = 1 SV = 2 | | | | | | |
| Prelamin-A/C OS = Homo sapiens OX = 9606 GN = LMNA | 74 | kDa | 48 | 149 | 20 | 39 |
| PE = 1 SV = 1 | | | | | | |
| Heat shock protein HSP 90-beta OS = Homo sapiens | 83 | kDa | 47 | 0 | 3 | 31 |
| OX = 9606 GN = HSP90AB1 PE = 1 SV = 4 | | | | | | |
| Ras GTPase-activating-like protein IQGAP1 OS = Homo | 189 | kDa | 47 | 0 | 11 | 36 |
| sapiens OX = 9606 GN = IQGAP1 PE = 1 SV = 1 | | | | | | |
| Heat shock cognate 71 kDa protein OS = Homo sapiens | 71 | kDa | 46 | 130 | 28 | 56 |
| OX = 9606 GN = HSPA8 PE = 1 SV = 1 | | | | | | |
| Annexin A2 OS = Homo sapiens OX = 9606 GN = ANXA2 | 39 | kDa | 43 | 111 | 5 | 40 |
| PE = 1 SV = 2 | | | | | | |
| Isoform 2 of Collagen alpha-1(XVIII) chain OS = Homo | 154 | kDa | 42 | 575 | 77 | 61 |
| sapiens OX = 9606 GN = COL18A1 | | | | | | |
| Myosin regulatory light polypeptide 9 OS = Homo sapiens | 20 | kDa | 42 | 380 | 131 | 81 |
| OX = 9606 GN = MYL9 PE = 1 SV = 4 | | | | | | |
| Isoform 6 of Agrin OS = Homo sapiens OX = 9606 | 215 | kDa | 41 | 168 | 42 | 70 |
| GN = AGRN | | | | | | |
| Histone H3.2 OS = Homo sapiens OX = 9606 | 15 | kDa | 40 | 0 | 17 | 25 |
| GN = HIST2H3A PE = 1 SV = 3 | | | | | | |
| Isoform 1 of Core histone macro-H2A.1 OS = Homo | 39 | kDa | 40 | 450 | 43 | 49 |
| sapiens OX = 9606 GN = H2AFY | | | | | | |
| ATP synthase subunit beta, mitochondrial OS = Homo | 57 | kDa | 39 | 94 | 14 | 45 |
| sapiens OX = 9606 GN = ATP5F1B PE = 1 SV = 3 | | | | | | |
| T-complex protein 1 subunit alpha OS = Homo sapiens | 60 | kDa | 39 | 0 | 25 | 36 |
| OX = 9606 GN = TCP1 PE = 1 SV = 1 | | | | | | |
| Isoform 2 of Heat shock protein HSP 90-alpha | 98 | kDa | 38 | 80 | 1 | 20 |
| OS = Homo sapiens GN = HSP90AA1 | | | | | | |
| Transitional endoplasmic reticulum ATPase OS = Homo | 89 | kDa | 38 | 86 | 1 | 37 |
| sapiens OX = 9606 GN = VCP PE = 1 SV = 4 | | | | | | |
| TREMBL: Q3KNV1; Q96GE1 Tax_Id = 9606 | 51 | kDa | 38 | 334 | 58 | 133 |
| Gene_Symbol = KRT7 keratin 7 | | | | | | |
| (Bos taurus) similar to alpha-2-macroglobulin isoform 1 | 164 | kDa | 37 | 14 | 9 | 13 |
| Heterogeneous nuclear ribonucleoprotein U OS = Homo | 91 | kDa | 35 | 64 | 12 | 29 |
| sapiens OX = 9606 GN = HNRNPU PE = 1 SV = 6 | | | | | | |
| Histone H3.3 OS = Homo sapiens OX = 9606 GN = H3F3A | 15 | kDa | 35 | 0 | 17 | 43 |
| PE = 1 SV = 2 | | | | | | |
| Microtubule-associated protein 4 OS = Homo sapiens | 121 | kDa | 35 | 73 | 8 | 26 |
| OX = 9606 GN = MAP4 PE = 1 SV = 3 | | | | | | |
| Beta-actin-like protein 2 OS = Homo sapiens OX = 9606 | 42 | kDa | 34 | 147 | 42 | 31 |
| GN = ACTBL2 PE = 1 SV = 2 | | | | | | |
| Keratin, type I cytoskeletal 10 OS = Homo sapiens | 59 | kDa | 34 | 464 | 32 | 37 |
| OX = 9606 GN = KRT10 PE = 1 SV = 6 | | | | | | |
| Ribosome-binding protein 1 OS = Homo sapiens | 152 | kDa | 34 | 80 | 17 | 22 |
| OX = 9606 GN = RRBP1 PE = 1 SV = 5 | | | | | | |
| Cytoskeleton-associated protein 4 OS = Homo sapiens | 66 | kDa | 33 | 139 | 30 | 35 |
| OX = 9606 GN = CKAP4 PE = 1 SV = 2 | | | | | | |
| DNA-dependent protein kinase catalytic subunit | 469 | kDa | 33 | 10 | 0 | 5 |
| OS = Homo sapiens OX = 9606 GN = PRKDC PE = 1 SV = 3 | | | | | | |
| Adenylyl cyclase-associated protein 1 OS = Homo sapiens | 52 | kDa | 32 | 33 | 2 | 37 |
| OX = 9606 GN = CAP1 PE = 1 SV = 5 | | | | | | |
| Keratin, type I cytoskeletal 9 OS = Homo sapiens | 62 | kDa | 31 | 643 | 61 | 50 |
| OX = 9606 GN = KRT9 PE = 1 SV = 3 | | | | | | |
| Laminin subunit alpha-5 OS = Homo sapiens OX = 9606 | 400 | kDa | 31 | 50 | 7 | 13 |
| GN = LAMA5 PE = 1 SV = 8 | | | | | | |
| (Bos taurus) similar to fibulin-1 C isoform 1 | 77 | kDa | 30 | 71 | 40 | 18 |
| 60S ribosomal protein L4 OS = Homo sapiens OX = 9606 | 48 | kDa | 30 | 75 | 26 | 25 |
| GN = RPL4 PE = 1 SV = 5 | | | | | | |
| Alpha-enolase OS = Homo sapiens OX = 9606 GN = ENO1 | 47 | kDa | 30 | 61 | 3 | 26 |
| PE = 1 SV = 2 | | | | | | |
| ATP synthase subunit alpha, mitochondrial OS = Homo | 60 | kDa | 30 | 52 | 12 | 34 |
| sapiens OX = 9606 GN = ATP5F1A PE = 1 SV = 1 | | | | | | |
| Heterogeneous nuclear ribonucleoprotein M OS = Homo | 78 | kDa | 30 | 114 | 12 | 30 |
| sapiens OX = 9606 GN = HNRNPM PE = 1 SV = 3 | | | | | | |
| Histone H2A.V OS = Homo sapiens OX = 9606 | 14 | kDa | 30 | 131 | 23 | 34 |
| GN = H2AFV PE = 1 SV = 3 | | | | | | |
| Lysyl oxidase homolog 2 OS = Homo sapiens OX = 9606 | 87 | kDa | 30 | 320 | 94 | 52 |
| GN = LOXL2 PE = 1 SV = 1 | | | | | | |
| MICOS complex subunit MIC60 OS = Homo sapiens | 84 | kDa | 30 | 98 | 20 | 36 |
| OX = 9606 GN = IMMT PE = 1 SV = 1 | | | | | | |
| SWISS-PROT: P12763 (Bos taurus) Alpha-2-HS- | 38 | kDa | 30 | 42 | 18 | 19 |
| glycoprotein precursor | | | | | | |
| Endoplasmin OS = Homo sapiens OX = 9606 | 92 | kDa | 29 | 71 | 3 | 22 |
| GN = HSP90B1 PE = 1 SV = 1 | | | | | | |
| TREMBL: Q0IIK2 (Bos taurus) Transferrin | 78 | kDa | 29 | 15 | 2 | 16 |
| Laminin subunit beta-1 OS = Homo sapiens OX = 9606 | 198 | kDa | 28 | 30 | 4 | 11 |
| GN = LAMB1 PE = 1 SV = 2 | | | | | | |
| T-complex protein 1 subunit beta OS = Homo sapiens | 57 | kDa | 27 | 117 | 19 | 31 |
| OX = 9606 GN = CCT2 PE = 1 SV = 4 | | | | | | |
| T-complex protein 1 subunit delta OS = Homo sapiens | 58 | kDa | 27 | 73 | 19 | 26 |
| OX = 9606 GN = CCT4 PE = 1 SV = 4 | | | | | | |
| Heterogeneous nuclear ribonucleoprotein K OS = Homo | 51 | kDa | 26 | 28 | 3 | 26 |
| sapiens OX = 9606 GN = HNRNPK PE = 1 SV = 1 | | | | | | |
| Bifunctional glutamate/proline--tRNA ligase OS = Homo | 171 | kDa | 25 | 20 | 0 | 1 |
| sapiens OX = 9606 GN = EPRS PE = 1 SV = 5 | | | | | | |
| Isoform 1 of Vinculin OS = Homo sapiens OX = 9606 | 117 | kDa | 25 | 25 | 1 | 26 |
| GN = VCL | | | | | | |
| Isoform 2 of MICOS complex subunit MIC60 OS = Homo | 83 | kDa | 25 | 99 | 17 | 34 |
| sapiens OX = 9606 GN = IMMT | | | | | | |
| T-complex protein 1 subunit theta OS = Homo sapiens | 60 | kDa | 25 | 100 | 21 | 30 |
| OX = 9606 GN = CCT8 PE = 1 SV = 4 | | | | | | |
| Isoform LCRMP-4 of Dihydropyrimidinase-related | 74 | kDa | 24 | 11 | 4 | 40 |
| protein 3 OS = Homo sapiens OX = 9606 GN = DPYSL3 | | | | | | |
| Thrombospondin-1 OS = Homo sapiens OX = 9606 | 129 | kDa | 24 | 103 | 3 | 1 |
| GN = THBS1 PE = 1 SV = 2 | | | | | | |
| Isoform Short of 14-3-3 protein beta/alpha OS = Homo | 28 | kDa | 23 | 18 | 2 | 14 |
| sapiens OX = 9606 GN = YWHAB | | | | | | |
| Isoform Smooth muscle of Myosin light polypeptide 6 | 17 | kDa | 23 | 204 | 37 | 28 |
| OS = Homo sapiens OX = 9606 GN = MYL6 | | | | | | |
| Keratin, type I cytoskeletal 18 OS = Homo sapiens | 48 | kDa | 23 | 376 | 99 | 120 |
| OX = 9606 GN = KRT18 PE = 1 SV = 2 | | | | | | |
| Keratin, type II cytoskeletal 8 OS = Homo sapiens | 54 | kDa | 23 | 1404 | 107 | 155 |
| OX = 9606 GN = KRT8 PE = 1 SV = 7 | | | | | | |
| Lamin-B1 OS = Homo sapiens OX = 9606 GN = LMNB1 | 66 | kDa | 23 | 67 | 7 | 21 |
| PE = 1 SV = 2 | | | | | | |
| L-lactate dehydrogenase A chain OS = Homo sapiens | 37 | kDa | 23 | 18 | 2 | 10 |
| GN = LDHA PE = 1 SV = 2 | | | | | | |
| Profilin-1 OS = Homo sapiens OX = 9606 GN = PFN1 PE = 1 | 15 | kDa | 23 | 0 | 6 | 17 |
| SV = 2 | | | | | | |
| Protein disulfide-isomerase OS = Homo sapiens OX = 9606 | 57 | kDa | 23 | 0 | 6 | 19 |
| GN = P4HB PE = 1 SV = 3 | | | | | | |
| T-complex protein 1 subunit gamma OS = Homo sapiens | 61 | kDa | 23 | 72 | 17 | 26 |
| GN = CCT3 PE = 1 SV = 4 | | | | | | |
| 40S ribosomal protein S15 OS = Homo sapiens OX = 9606 | 17 | kDa | 22 | 0 | 26 | 38 |
| GN = RPS15 PE = 1 SV = 2 | | | | | | |
| ATP-citrate synthase OS = Homo sapiens OX = 9606 | 121 | kDa | 22 | 20 | 2 | 18 |
| GN = ACLY PE = 1 SV = 3 | | | | | | |
| Isoform 2 of Transgelin-2 OS = Homo sapiens OX = 9606 | 24 | kDa | 22 | 47 | 1 | 20 |
| GN = TAGLN2 | | | | | | |
| Versican core protein OS = Homo sapiens OX = 9606 | 373 | kDa | 22 | 188 | 33 | 27 |
| GN = VCAN PE = 1 SV = 3 | | | | | | |
| 60S acidic ribosomal protein P0 OS = Homo sapiens | 34 | kDa | 21 | 56 | 14 | 19 |
| OX = 9606 GN = RPLP0 PE = 1 SV = 1 | | | | | | |
| Histone H1.5 OS = Homo sapiens OX = 9606 | 23 | kDa | 21 | 53 | 11 | 20 |
| GN = HIST1H1B PE = 1 SV = 3 | | | | | | |
| Importin subunit beta-1 OS = Homo sapiens OX = 9606 | 97 | kDa | 21 | 19 | 1 | 6 |
| GN = KPNB1 PE = 1 SV = 2 | | | | | | |
| Isoform 2 of Fructose-bisphosphate aldolase A | 45 | kDa | 21 | 37 | 0 | 21 |
| OS = Homo sapiens OX = 9606 GN = ALDOA | | | | | | |
| Microtubule-associated protein 1B OS = Homo sapiens | 271 | kDa | 21 | 104 | 0 | 22 |
| OX = 9606 GN = MAP1B PE = 1 SV = 2 | | | | | | |
| TREMBL: Q3SX09 (Bos taurus) similar to HBG protein | 22 | kDa | 21 | 26 | 11 | 9 |
| 5′-nucleotidase OS = Homo sapiens OX = 9606 GN = NT5E | 63 | kDa | 20 | 129 | 21 | 17 |
| PE = 1 SV = 1 | | | | | | |
| 60S ribosomal protein L3 OS = Homo sapiens OX = 9606 | 46 | kDa | 20 | 65 | 15 | 24 |
| GN = RPL3 PE = 1 SV = 2 | | | | | | |
| 60S ribosomal protein L6 OS = Homo sapiens OX = 9606 | 33 | kDa | 20 | 0 | 19 | 25 |
| GN = RPL6 PE = 1 SV = 3 | | | | | | |
| ATP-dependent 6-phosphofructokinase, platelet type | 86 | kDa | 20 | 9 | 0 | 18 |
| OS = Homo sapiens OX = 9606 GN = PFKP PE = 1 SV = 2 | | | | | | |
| Collagen alpha-1(I) chain OS = Homo sapiens OX = 9606 | 139 | kDa | 20 | 41 | 43 | 27 |
| GN = COL1A1 PE = 1 SV = 5 | | | | | | |
| T-complex protein 1 subunit eta OS = Homo sapiens | 59 | kDa | 20 | 72 | 15 | 22 |
| OX = 9606 GN = CCT7 PE = 1 SV = 2 | | | | | | |
| Trifunctional enzyme subunit alpha, mitochondrial | 83 | kDa | 20 | 78 | 16 | 13 |
| OS = Homo sapiens OX = 9606 GN = HADHA PE = 1 SV = 2 | | | | | | |
| 40S ribosomal protein S7 OS = Homo sapiens OX = 9606 | 22 | kDa | 19 | 104 | 25 | 28 |
| GN = RPS7 PE = 1 SV = 1 | | | | | | |
| 60S ribosomal protein L9 OS = Homo sapiens OX = 9606 | 22 | kDa | 19 | 0 | 14 | 21 |
| GN = RPL9 PE = 1 SV = 1 | | | | | | |
| Actin-related protein 3 OS = Homo sapiens OX = 9606 | 47 | kDa | 19 | 0 | 16 | 18 |
| GN = ACTR3 PE = 1 SV = 3 | | | | | | |
| Annexin A5 OS = Homo sapiens OX = 9606 GN = ANXA5 | 36 | kDa | 19 | 0 | 0 | 15 |
| PE = 1 SV = 2 | | | | | | |
| Calpain-2 catalytic subunit OS = Homo sapiens OX = 9606 | 80 | kDa | 19 | 4 | 0 | 8 |
| GN = CAPN2 PE = 1 SV = 6 | | | | | | |
| Heterogeneous nuclear ribonucleoprotein A1 OS = Homo | 39 | kDa | 19 | 55 | 10 | 23 |
| sapiens OX = 9606 GN = HNRNPA1 PE = 1 SV = 5 | | | | | | |
| Integrin beta-1 OS = Homo sapiens OX = 9606 GN = ITGB1 | 88 | kDa | 19 | 16 | 4 | 18 |
| PE = 1 SV = 2 | | | | | | |
| Interleukin enhancer-binding factor 2 OS = Homo sapiens | 43 | kDa | 19 | 0 | 4 | 15 |
| OX = 9606 GN = ILF2 PE = 1 SV = 2 | | | | | | |
| Isoform 2 of Nidogen-2 OS = Homo sapiens OX = 9606 | 141 | kDa | 19 | 55 | 0 | 3 |
| GN = NID2 | | | | | | |
| Matrin-3 OS = Homo sapiens OX = 9606 GN = MATR3 | 95 | kDa | 19 | 46 | 5 | 20 |
| PE = 1 SV = 2 | | | | | | |
| T-complex protein 1 subunit zeta OS = Homo sapiens | 58 | kDa | 19 | 64 | 23 | 28 |
| OX = 9606 GN = CCT6A PE = 1 SV = 3 | | | | | | |
| 40S ribosomal protein S2 OS = Homo sapiens OX = 9606 | 31 | kDa | 18 | 0 | 14 | 17 |
| GN = RPS2 PE = 1 SV = 2 | | | | | | |
| 40S ribosomal protein S3 OS = Homo sapiens OX = 9606 | 27 | kDa | 18 | 82 | 19 | 20 |
| GN = RPS3 PE = 1 SV = 2 | | | | | | |
| Collagen alpha-2(IV) chain OS = Homo sapiens OX = 9606 | 168 | kDa | 18 | 209 | 44 | 33 |
| GN = COL4A2 PE = 1 SV = 4 | | | | | | |
| Epiplakin OS = Homo sapiens OX = 9606 GN = EPPK1 | 556 | kDa | 18 | 22 | 1 | 39 |
| PE = 1 SV = 3 | | | | | | |
| Heat shock 70 kDa protein 1A OS = Homo sapiens | 70 | kDa | 18 | 31 | 0 | 23 |
| OX = 9606 GN = HSPA1A PE = 1 SV = 1 | | | | | | |
| Heterogeneous nuclear ribonucleoproteins C1/C2 | 34 | kDa | 18 | 52 | 6 | 22 |
| OS = Homo sapiens OX = 9606 GN = HNRNPC PE = 1 SV = 4 | | | | | | |
| Isoform 2 of Gelsolin OS = Homo sapiens OX = 9606 | 81 | kDa | 18 | 87 | 36 | 24 |
| GN = GSN | | | | | | |
| Isoform 5 of Septin-9 OS = Homo sapiens OX = 9606 | 65 | kDa | 18 | 42 | 1 | 16 |
| GN = SEPT9 | | | | | | |
| Laminin subunit gamma-1 OS = Homo sapiens OX = 9606 | 178 | kDa | 18 | 48 | 0 | 6 |
| GN = LAMC1 PE = 1 SV = 3 | | | | | | |
| Nucleolin OS = Homo sapiens OX = 9606 GN = NCL PE = 1 | 77 | kDa | 18 | 58 | 8 | 16 |
| SV = 3 | | | | | | |
| Voltage-dependent anion-selective channel protein 1 | 31 | kDa | 18 | 50 | 4 | 19 |
| OS = Homo sapiens OX = 9606 GN = VDAC1 PE = 1 SV = 2 | | | | | | |
| X-ray repair cross-complementing protein 6 OS = Homo | 70 | kDa | 18 | 33 | 2 | 14 |
| sapiens OX = 9606 GN = XRCC6 PE = 1 SV = 2 | | | | | | |
| Actin-related protein 2 OS = Homo sapiens OX = 9606 | 45 | kDa | 17 | 43 | 17 | 28 |
| GN = ACTR2 PE = 1 SV = 1 | | | | | | |
| Heat shock protein beta-1 OS = Homo sapiens OX = 9606 | 23 | kDa | 17 | 0 | 9 | 35 |
| GN = HSPB1 PE = 1 SV = 2 | | | | | | |
| Histone H1.4 OS = Homo sapiens OX = 9606 | 22 | kDa | 17 | 14 | 10 | 13 |
| GN = HIST1H1E PE = 1 SV = 2 | | | | | | |
| Isoform 2 of AP-2 complex subunit beta OS = Homo | 106 | kDa | 17 | 33 | 1 | 15 |
| sapiens OX = 9606 GN = AP2B1 | | | | | | |
| Isoform 2 of Calnexin OS = Homo sapiens OX = 9606 | 72 | kDa | 17 | 21 | 1 | 19 |
| GN = CANX | | | | | | |
| Isoform 2 of Dolichyl-diphosphooligosaccharide--protein | 68 | kDa | 17 | 35 | 8 | 25 |
| glycosyltransferase subunit 2 OS = Homo sapiens | | | | | | |
| OX = 9606 GN = RPN2 | | | | | | |
| Moesin OS = Homo sapiens OX = 9606 GN = MSN PE = 1 | 68 | kDa | 17 | 61 | 3 | 15 |
| SV = 3 | | | | | | |
| Protein disulfide-isomerase A3 OS = Homo sapiens | 57 | kDa | 17 | 26 | 0 | 14 |
| OX = 9606 GN = PDIA3 PE = 1 SV = 4 | | | | | | |
| Stress-70 protein, mitochondrial OS = Homo sapiens | 74 | kDa | 17 | 33 | 0 | 10 |
| OX = 9606 GN = HSPA9 PE = 1 SV = 2 | | | | | | |
| SWISS-PROT: P34955 (Bos taurus) Alpha-1- | 46 | kDa | 17 | 66 | 19 | 16 |
| antiproteinase precursor | | | | | | |
| T-complex protein 1 subunit epsilon OS = Homo sapiens | 60 | kDa | 17 | 110 | 18 | 22 |
| OX = 9606 GN = CCT5 PE = 1 SV = 1 | | | | | | |
| 14-3-3 protein zeta/delta OS = Homo sapiens OX = 9606 | 28 | kDa | 16 | 35 | 4 | 19 |
| GN = YWHAZ PE = 1 SV = 1 | | | | | | |
| 40S ribosomal protein S4, X isoform OS = Homo sapiens | 30 | kDa | 16 | 77 | 20 | 20 |
| OX = 9606 GN = RPS4X PE = 1 SV = 2 | | | | | | |
| 60S ribosomal protein L10 OS = Homo sapiens OX = 9606 | 25 | kDa | 16 | 27 | 7 | 12 |
| GN = RPL10 PE = 1 SV = 4 | | | | | | |
| Ezrin OS = Homo sapiens OX = 9606 GN = EZR PE = 1 | 69 | kDa | 16 | 43 | 4 | 23 |
| SV = 4 | | | | | | |
| High mobility group protein HMG-I/HMG-Y OS = Homo | 12 | kDa | 16 | 52 | 7 | 11 |
| sapiens OX = 9606 GN = HMGA1 PE = 1 SV = 3 | | | | | | |
| Hypoxia up-regulated protein 1 OS = Homo sapiens | 111 | kDa | 16 | 37 | 0 | 18 |
| OX = 9606 GN = HYOU1 PE = 1 SV = 1 | | | | | | |
| Isoform 2 of Ubiquitin-like modifier-activating enzyme 1 | 114 | kDa | 16 | 21 | 0 | 5 |
| OS = Homo sapiens OX = 9606 GN = UBA1 | | | | | | |
| Isoform 3 of Heterogeneous nuclear ribonucleoprotein Q | 63 | kDa | 16 | 37 | 5 | 20 |
| OS = Homo sapiens OX = 9606 GN = SYNCRIP | | | | | | |
| Nidogen-1 OS = Homo sapiens OX = 9606 GN = NID1 | 136 | kDa | 16 | 56 | 1 | 7 |
| PE = 1 SV = 3 | | | | | | |
| 14-3-3 protein epsilon OS = Homo sapiens OX = 9606 | 29 | kDa | 15 | 26 | 3 | 16 |
| GN = YWHAE PE = 1 SV = 1 | | | | | | |
| 40S ribosomal protein S5 OS = Homo sapiens OX = 9606 | 23 | kDa | 15 | 0 | 10 | 12 |
| GN = RPS5 PE = 1 SV = 4 | | | | | | |
| 60S ribosomal protein L5 OS = Homo sapiens OX = 9606 | 34 | kDa | 15 | 0 | 16 | 16 |
| GN = RPL5 PE = 1 SV = 3 | | | | | | |
| 60S ribosomal protein L7 OS = Homo sapiens OX = 9606 | 29 | kDa | 15 | 61 | 14 | 14 |
| GN = RPL7 PE = 1 SV = 1 | | | | | | |
| Caprin-1 OS = Homo sapiens OX = 9606 GN = CAPRIN1 | 78 | kDa | 15 | 18 | 0 | 14 |
| PE = 1 SV = 2 | | | | | | |
| Catenin alpha-1 OS = Homo sapiens OX = 9606 | 100 | kDa | 15 | 13 | 0 | 12 |
| GN = CTNNA1 PE = 1 SV = 1 | | | | | | |
| Eukaryotic initiation factor 4A-I OS = Homo sapiens | 46 | kDa | 15 | 28 | 2 | 8 |
| OX = 9606 GN = EIF4A1 PE = 1 SV = 1 | | | | | | |
| Heterogeneous nuclear ribonucleoproteins A2/B1 | 37 | kDa | 15 | 58 | 6 | 20 |
| OS = Homo sapiens OX = 9606 GN = HNRNPA2B1 PE = 1 | | | | | | |
| SV = 2 | | | | | | |
| Isoform 2 of Coatomer subunit alpha OS = Homo sapiens | 139 | kDa | 15 | 8 | 0 | 0 |
| OX = 9606 GN = COPA | | | | | | |
| Isoform 2 of Protein disulfide-isomerase A6 OS = Homo | 54 | kDa | 15 | 23 | 0 | 15 |
| sapiens OX = 9606 GN = PDIA6 | | | | | | |
| Isoform 3 of Septin-2 OS = Homo sapiens OX = 9606 | 43 | kDa | 15 | 55 | 6 | 16 |
| GN = SEPT2 | | | | | | |
| Staphylococcal nuclease domain-containing protein 1 | 102 | kDa | 15 | 33 | 0 | 6 |
| OS = Homo sapiens OX = 9606 GN = SND1 PE = 1 SV = 1 | | | | | | |
| Triosephosphate isomerase OS = Homo sapiens OX = 9606 | 31 | kDa | 15 | 15 | 0 | 11 |
| GN = TPI1 PE = 1 SV = 3 | | | | | | |
| UDP-glucose 6-dehydrogenase OS = Homo sapiens | 55 | kDa | 15 | 24 | 2 | 15 |
| OX = 9606 GN = UGDH PE = 1 SV = 1 | | | | | | |
| ADP/ATP translocase 2 OS = Homo sapiens OX = 9606 | 33 | kDa | 14 | 27 | 9 | 17 |
| GN = SLC25A5 PE = 1 SV = 7 | | | | | | |
| ATP-dependent RNA helicase A OS = Homo sapiens | 141 | kDa | 14 | 25 | 1 | 9 |
| OX = 9606 GN = DHX9 PE = 1 SV = 4 | | | | | | |
| Cofilin-1 OS = Homo sapiens OX = 9606 GN = CFL1 PE = 1 | 19 | kDa | 14 | 0 | 4 | 11 |
| SV = 3 | | | | | | |
| eIF-2-alpha kinase activator GCN1 OS = Homo sapiens | 293 | kDa | 14 | 0 | 0 | 6 |
| OX = 9606 GN = GCN1 PE = 1 SV = 6 | | | | | | |
| Isoform 2 of Kinectin OS = Homo sapiens OX = 9606 | 150 | kDa | 14 | 62 | 1 | 8 |
| GN = KTN1 | | | | | | |
| Isoform 2 of Spliceosome RNA helicase DDX39B | 51 | kDa | 14 | 25 | 1 | 12 |
| OS = Homo sapiens OX = 9606 GN = DDX39B | | | | | | |
| Isoleucine--tRNA ligase, cytoplasmic OS = Homo sapiens | 145 | kDa | 14 | 0 | 0 | 0 |
| OX = 9606 GN = IARS PE = 1 SV = 2 | | | | | | |
| Keratin, type II cytoskeletal 2 epidermal OS = Homo | 65 | kDa | 14 | 123 | 28 | 21 |
| sapiens OX = 9606 GN = KRT2 PE = 1 SV = 2 | | | | | | |
| Ribonuclease inhibitor OS = Homo sapiens OX = 9606 | 50 | kDa | 14 | 0 | 1 | 8 |
| GN = RNH1 PE = 1 SV = 2 | | | | | | |
| SWISS-PROT: P02070 (Bos taurus) Hemoglobin subunit | 16 | kDa | 14 | 0 | 0 | 0 |
| beta | | | | | | |
| SWISS-PROT: Q9DCV7 Tax_Id = 10090 | 51 | kDa | 14 | 0 | 14 | 18 |
| Gene_Symbol = Krt7 Keratin, type II cytoskeletal 7 | | | | | | |
| Tubulointerstitial nephritis antigen-like OS = Homo | 52 | kDa | 14 | 173 | 78 | 38 |
| sapiens OX = 9606 GN = TINAGL1 PE = 1 SV = 1 | | | | | | |
| 26S proteasome non-ATPase regulatory subunit 2 | 100 | kDa | 13 | 18 | 0 | 10 |
| OS = Homo sapiens OX = 9606 GN = PSMD2 PE = 1 SV = 3 | | | | | | |
| 40S ribosomal protein S8 OS = Homo sapiens OX = 9606 | 24 | kDa | 13 | 0 | 9 | 15 |
| GN = RPS8 PE = 1 SV = 2 | | | | | | |
| 60S ribosomal protein L23 OS = Homo sapiens OX = 9606 | 15 | kDa | 13 | 0 | 14 | 10 |
| GN = RPL23 PE = 1 SV = 1 | | | | | | |
| 60S ribosomal protein L7a OS = Homo sapiens OX = 9606 | 30 | kDa | 13 | 68 | 12 | 14 |
| GN = RPL7A PE = 1 SV = 2 | | | | | | |
| ADP/ATP translocase 3 OS = Homo sapiens OX = 9606 | 33 | kDa | 13 | 0 | 8 | 18 |
| GN = SLC25A6 PE = 1 SV = 4 | | | | | | |
| Arginine--tRNA ligase, cytoplasmic OS = Homo sapiens | 75 | kDa | 13 | 33 | 0 | 5 |
| OX = 9606 GN = RARS PE = 1 SV = 2 | | | | | | |
| Calpain small subunit 1 OS = Homo sapiens OX = 9606 | 28 | kDa | 13 | 0 | 0 | 22 |
| GN = CAPNS1 PE = 1 SV = 1 | | | | | | |
| Glycine--tRNA ligase OS = Homo sapiens OX = 9606 | 83 | kDa | 13 | 0 | 1 | 6 |
| GN = GARS PE = 1 SV = 3 | | | | | | |
| Isoform 2 of Extended synaptotagmin-1 OS = Homo | 124 | kDa | 13 | 21 | 1 | 17 |
| sapiens OX = 9606 GN = ESYT1 | | | | | | |
| Isoform 2 of Nuclear mitotic apparatus protein 1 | 237 | kDa | 13 | 18 | 0 | 2 |
| OS = Homo sapiens OX = 9606 GN = NUMA1 | | | | | | |
| Isoform 2 of Tropomyosin beta chain OS = Homo sapiens | 33 | kDa | 13 | 122 | 33 | 24 |
| OX = 9606 GN = TPM2 | | | | | | |
| Isoform 7 of Interleukin enhancer-binding factor 3 | 96 | kDa | 13 | 31 | 0 | 13 |
| OS = Homo sapiens GN = ILF3 | | | | | | |
| Isoform Beta-1 of DNA topoisomerase 2-beta OS = Homo | 183 | kDa | 13 | 97 | 3 | 3 |
| sapiens OX = 9606 GN = TOP2B | | | | | | |
| Lamin-B2 OS = Homo sapiens OX = 9606 GN = LMNB2 | 70 | kDa | 13 | 53 | 8 | 19 |
| PE = 1 SV = 4 | | | | | | |
| L-lactate dehydrogenase B chain OS = Homo sapiens | 37 | kDa | 13 | 0 | 0 | 6 |
| OX = 9606 GN = LDHB PE = 1 SV = 2 | | | | | | |
| Nucleoprotein TPR OS = Homo sapiens OX = 9606 | 267 | kDa | 13 | 15 | 1 | 3 |
| GN = TPR PE = 1 SV = 3 | | | | | | |
| Receptor of activated protein C kinase 1 OS = Homo | 35 | kDa | 13 | 0 | 1 | 12 |
| sapiens OX = 9606 GN = RACK1 PE = 1 SV = 3 | | | | | | |
| Serine protease HTRA1 OS = Homo sapiens OX = 9606 | 51 | kDa | 13 | 0 | 15 | 1 |
| GN = HTRA1 PE = 1 SV = 1 | | | | | | |
| Serine/threonine-protein phosphatase 2A 65 kDa | 65 | kDa | 13 | 0 | 0 | 10 |
| regulatory subunit A alpha isoform OS = Homo sapiens | | | | | | |
| OX = 9606 GN = PPP2R1A PE = 1 SV = 4 | | | | | | |
| X-ray repair cross-complementing protein 5 OS = Homo | 83 | kDa | 13 | 0 | 2 | 15 |
| sapiens OX = 9606 GN = XRCC5 PE = 1 SV = 3 | | | | | | |
| 14-3-3 protein theta OS = Homo sapiens OX = 9606 | 28 | kDa | 12 | 0 | 2 | 12 |
| GN = YWHAQ PE = 1 SV = 1 | | | | | | |
| 40S ribosomal protein S12 OS = Homo sapiens OX = 9606 | 15 | kDa | 12 | 0 | 1 | 9 |
| GN = RPS12 PE = 1 SV = 3 | | | | | | |
| Cytoplasmic dynein 1 light intermediate chain 2 | 54 | kDa | 12 | 12 | 3 | 19 |
| OS = Homo sapiens OX = 9606 GN = DYNC1LI2 PE = 1 | | | | | | |
| SV = 1 | | | | | | |
| Glutathione S-transferase P OS = Homo sapiens OX = 9606 | 23 | kDa | 12 | 0 | 2 | 13 |
| GN = GSTP1 PE = 1 SV = 2 | | | | | | |
| Isoform 2 of Septin-7 OS = Homo sapiens OX = 9606 | 51 | kDa | 12 | 31 | 0 | 12 |
| GN = SEPT7 | | | | | | |
| Isoform 3 of Unconventional myosin-Ic OS = Homo | 120 | kDa | 12 | 64 | 9 | 8 |
| sapiens OX = 9606 GN = MYO1C | | | | | | |
| Peptidyl-prolyl cis-trans isomerase A OS = Homo sapiens | 18 | kDa | 12 | 36 | 3 | 11 |
| OX = 9606 GN = PPIA PE = 1 SV = 2 | | | | | | |
| Polyadenylate-binding protein 1 OS = Homo sapiens | 71 | kDa | 12 | 22 | 0 | 15 |
| OX = 9606 GN = PABPC1 PE = 1 SV = 2 | | | | | | |
| Rho-related GTP-binding protein RhoC OS = Homo | 22 | kDa | 12 | 0 | 5 | 12 |
| sapiens OX = 9606 GN = RHOC PE = 1 SV = 1 | | | | | | |
| Ribosomal L1 domain-containing protein 1 OS = Homo | 55 | kDa | 12 | 18 | 8 | 12 |
| sapiens OX = 9606 GN = RSL1D1 PE = 1 SV = 3 | | | | | | |
| Transgelin OS = Homo sapiens OX = 9606 GN = TAGLN | 23 | kDa | 12 | 41 | 10 | 23 |
| PE = 1 SV = 4 | | | | | | |
| 14-3-3 protein gamma OS = Homo sapiens OX = 9606 | 28 | kDa | 11 | 0 | 1 | 14 |
| GN = YWHAG PE = 1 SV = 2 | | | | | | |
| 26S proteasome regulatory subunit 6A OS = Homo | 47 | kDa | 11 | 0 | 0 | 17 |
| sapiens OX = 9606 GN = PSMC3 PE = 1 SV = 1 | | | | | | |
| 40S ribosomal protein S17 OS = Homo sapiens OX = 9606 | 16 | kDa | 11 | 67 | 20 | 16 |
| GN = RPS17 PE = 1 SV = 2 | | | | | | |
| 60S ribosomal protein L10a OS = Homo sapiens | 25 | kDa | 11 | 0 | 11 | 17 |
| OX = 9606 GN = RPL10A PE = 1 SV = 2 | | | | | | |
| 60S ribosomal protein L12 OS = Homo sapiens OX = 9606 | 18 | kDa | 11 | 36 | 10 | 9 |
| GN = RPL12 PE = 1 SV = 1 | | | | | | |
| ADP-ribosylation factor 4 OS = Homo sapiens OX = 9606 | 21 | kDa | 11 | 0 | 8 | 14 |
| GN = ARF4 PE = 1 SV = 3 | | | | | | |
| Annexin A6 OS = Homo sapiens OX = 9606 GN = ANXA6 | 76 | kDa | 11 | 15 | 0 | 9 |
| PE = 1 SV = 3 | | | | | | |
| ATP synthase subunit O, mitochondrial OS = Homo | 23 | kDa | 11 | 0 | 5 | 10 |
| sapiens OX = 9606 GN = ATP5PO PE = 1 SV = 1 | | | | | | |
| Core histone macro-H2A.2 OS = Homo sapiens OX = 9606 | 40 | kDa | 11 | 226 | 28 | 21 |
| GN = H2AFY2 PE = 1 SV = 3 | | | | | | |
| Dolichyl-diphosphooligosaccharide--protein | 69 | kDa | 11 | 43 | 7 | 18 |
| glycosyltransferase subunit 1 OS = Homo sapiens | | | | | | |
| OX = 9606 GN = RPN1 PE = 1 SV = 1 | | | | | | |
| Elongation factor 1-gamma OS = Homo sapiens OX = 9606 | 50 | kDa | 11 | 29 | 0 | 8 |
| GN = EEF1G PE = 1 SV = 3 | | | | | | |
| Guanine nucleotide-binding protein G(i) subunit alpha-2 | 40 | kDa | 11 | 45 | 6 | 8 |
| OS = Homo sapiens OX = 9606 GN = GNAI2 PE = 1 SV = 3 | | | | | | |
| Heterogeneous nuclear ribonucleoprotein R OS = Homo | 71 | kDa | 11 | 17 | 4 | 12 |
| sapiens OX = 9606 GN = HNRNPR PE = 1 SV = 1 | | | | | | |
| Isoform 2 of Probable ATP-dependent RNA helicase | 72 | kDa | 11 | 17 | 0 | 9 |
| DDX17 OS = Homo sapiens OX = 9606 GN = DDX17 | | | | | | |
| Isoform 3 of Tropomyosin alpha-1 chain OS = Homo | 33 | kDa | 11 | 181 | 46 | 37 |
| sapiens OX = 9606 GN = TPM1 | | | | | | |
| Isoform 4 of Caldesmon OS = Homo sapiens OX = 9606 | 63 | kDa | 11 | 51 | 10 | 32 |
| GN = CALD1 | | | | | | |
| Leucine--tRNA ligase, cytoplasmic OS = Homo sapiens | 134 | kDa | 11 | 3 | 0 | 0 |
| GN = LARS PE = 1 SV = 2 | | | | | | |
| Phosphoglycerate kinase 1 OS = Homo sapiens OX = 9606 | 45 | kDa | 11 | 10 | 0 | 7 |
| GN = PGK1 PE = 1 SV = 3 | | | | | | |
| Polypyrimidine tract-binding protein 1 OS = Homo | 57 | kDa | 11 | 33 | 1 | 20 |
| sapiens OX = 9606 GN = PTBP1 PE = 1 SV = 1 | | | | | | |
| Rab GDP dissociation inhibitor beta OS = Homo sapiens | 51 | kDa | 11 | 17 | 0 | 10 |
| OX = 9606 GN = GDI2 PE = 1 SV = 2 | | | | | | |
| Reticulon-4 OS = Homo sapiens GN = RTN4 PE = 1 SV = 2 | 130 | kDa | 11 | 15 | 0 | 9 |
| Serine hydroxymethyltransferase, mitochondrial | 56 | kDa | 11 | 6 | 0 | 3 |
| OS = Homo sapiens GN = SHMT2 PE = 1 SV = 3 | | | | | | |
| Serine/threonine-protein phosphatase PP1-alpha catalytic | 38 | kDa | 11 | 23 | 3 | 10 |
| subunit OS = Homo sapiens OX = 9606 GN = PPP1CA | | | | | | |
| PE = 1 SV = 1 | | | | | | |
| Splicing factor, proline- and glutamine-rich OS = Homo | 76 | kDa | 11 | 43 | 5 | 18 |
| sapiens OX = 9606 GN = SFPQ PE = 1 SV = 2 | | | | | | |
| SWISS-PROT: Q3MHN5 (Bos taurus) Vitamin D- | 53 | kDa | 11 | 15 | 2 | 4 |
| binding protein precursor | | | | | | |
| Tenascin OS = Homo sapiens GN = TNC PE = 1 SV = 3 | 241 | kDa | 11 | 409 | 60 | 72 |
| Threonine--tRNA ligase, cytoplasmic OS = Homo sapiens | 83 | kDa | 11 | 10 | 0 | 3 |
| OX = 9606 GN = TARS PE = 1 SV = 3 | | | | | | |
| Tropomyosin alpha-4 chain OS = Homo sapiens OX = 9606 | 29 | kDa | 11 | 89 | 21 | 20 |
| GN = TPM4 PE = 1 SV = 3 | | | | | | |
| 14-3-3 protein eta OS = Homo sapiens OX = 9606 | 28 | kDa | 10 | 0 | 1 | 10 |
| GN = YWHAH PE = 1 SV = 4 | | | | | | |
| 26S proteasome regulatory subunit 6B OS = Homo sapiens | 47 | kDa | 10 | 13 | 3 | 10 |
| OX = 9606 GN = PSMC4 PE = 1 SV = 2 | | | | | | |
| 60S ribosomal protein L18 OS = Homo sapiens | 19 | kDa | 10 | 26 | 7 | 9 |
| GN = RPL18 PE = 1 SV = 1 | | | | | | |
| Actin-related protein 2/3 complex subunit 2 OS = Homo | 34 | kDa | 10 | 53 | 14 | 11 |
| sapiens OX = 9606 GN = ARPC2 PE = 1 SV = 1 | | | | | | |
| A-kinase anchor protein 12 OS = Homo sapiens OX = 9606 | 191 | kDa | 10 | 46 | 0 | 5 |
| GN = AKAP12 PE = 1 SV = 4 | | | | | | |
| Asparagine--tRNA ligase, cytoplasmic OS = Homo | 63 | kDa | 10 | 10 | 0 | 1 |
| sapiens OX = 9606 GN = NARS PE = 1 SV = 1 | | | | | | |
| Aspartate--tRNA ligase, cytoplasmic OS = Homo sapiens | 57 | kDa | 10 | 22 | 3 | 9 |
| OX = 9606 GN = DARS PE = 1 SV = 2 | | | | | | |
| Dolichyl-diphosphooligosaccharide--protein | 51 | kDa | 10 | 13 | 6 | 17 |
| glycosyltransferase 48 kDa subunit OS = Homo sapiens | | | | | | |
| OX = 9606 GN = DDOST PE = 1 SV = 4 | | | | | | |
| Erlin-2 OS = Homo sapiens OX = 9606 GN = ERLIN2 PE = 1 | 38 | kDa | 10 | 39 | 4 | 11 |
| SV = 1 | | | | | | |
| Fatty acid synthase OS = Homo sapiens OX = 9606 | 273 | kDa | 10 | 0 | 0 | 2 |
| GN = FASN PE = 1 SV = 3 | | | | | | |
| Heterogeneous nuclear ribonucleoprotein A3 OS = Homo | 40 | kDa | 10 | 25 | 7 | 8 |
| sapiens OX = 9606 GN = HNRNPA3 PE = 1 SV = 2 | | | | | | |
| Isoform 3 of Exportin-2 OS = Homo sapiens OX = 9606 | 108 | kDa | 10 | 2 | 2 | 3 |
| GN = CSE1L | | | | | | |
| Isoform B of AP-1 complex subunit beta-1 OS = Homo | 104 | kDa | 10 | 0 | 0 | 11 |
| sapiens OX = 9606 GN = AP1B1 | | | | | | |
| Leucine-rich PPR motif-containing protein, | 158 | kDa | 10 | 5 | 0 | 0 |
| mitochondrial OS = Homo sapiens OX = 9606 | | | | | | |
| GN = LRPPRC PE = 1 SV = 3 | | | | | | |
| Non-POU domain-containing octamer-binding protein | 54 | kDa | 10 | 44 | 3 | 20 |
| OS = Homo sapiens OX = 9606 GN = NONO PE = 1 SV = 4 | | | | | | |
| Nucleolar protein 56 OS = Homo sapiens OX = 9606 | 66 | kDa | 10 | 18 | 1 | 2 |
| GN = NOP56 PE = 1 SV = 4 | | | | | | |
| Peroxidasin homolog OS = Homo sapiens OX = 9606 | 165 | kDa | 10 | 146 | 36 | 28 |
| GN = PXDN PE = 1 SV = 2 | | | | | | |
| Stomatin-like protein 2, mitochondrial OS = Homo sapiens | 39 | kDa | 10 | 18 | 3 | 10 |
| OX = 9606 GN = STOML2 PE = 1 SV = 1 | | | | | | |
| SWISS-PROT: P01966 (Bos taurus) Hemoglobin subunit | 15 | kDa | 10 | 34 | 12 | 10 |
| alpha | | | | | | |
| SWISS-PROT: Q3SZ57 (Bos taurus) Alpha-fetoprotein | 69 | kDa | 10 | 11 | 6 | 7 |
| precursor | | | | | | |
| Transforming protein RhoA OS = Homo sapiens | 22 | kDa | 10 | 0 | 4 | 10 |
| OX = 9606 GN = RHOA PE = 1 SV = 1 | | | | | | |
| 40S ribosomal protein SA OS = Homo sapiens OX = 9606 | 33 | kDa | 9 | 0 | 1 | 10 |
| GN = RPSA PE = 1 SV = 1 | | | | | | |
| 60S ribosomal protein L13 OS = Homo sapiens OX = 9606 | 24 | kDa | 9 | 32 | 12 | 12 |
| GN = RPL13 PE = 1 SV = 4 | | | | | | |
| 60S ribosomal protein L8 OS = Homo sapiens OX = 9606 | 28 | kDa | 9 | 0 | 7 | 9 |
| GN = RPL8 PE = 1 SV = 2 | | | | | | |
| Aldehyde dehydrogenase X, mitochondrial OS = Homo | 57 | kDa | 9 | 14 | 4 | 11 |
| sapiens OX = 9606 GN = ALDH1B1 PE = 1 SV = 3 | | | | | | |
| ATP-dependent RNA helicase DDX3X OS = Homo | 73 | kDa | 9 | 18 | 5 | 11 |
| sapiens OX = 9606 GN = DDX3X PE = 1 SV = 3 | | | | | | |
| Calreticulin OS = Homo sapiens OX = 9606 GN = CALR | 48 | kDa | 9 | 44 | 4 | 11 |
| PE = 1 SV = 1 | | | | | | |
| D-3-phosphoglycerate dehydrogenase OS = Homo sapiens | 57 | kDa | 9 | 0 | 0 | 2 |
| OX = 9606 GN = PHGDH PE = 1 SV = 4 | | | | | | |
| F-actin-capping protein subunit alpha-1 OS = Homo | 33 | kDa | 9 | 0 | 18 | 15 |
| sapiens OX = 9606 GN = CAPZA1 PE = 1 SV = 3 | | | | | | |
| Galectin-1 OS = Homo sapiens OX = 9606 GN = LGALS1 | 15 | kDa | 9 | 0 | 3 | 6 |
| PE = 1 SV = 2 | | | | | | |
| GTP-binding nuclear protein Ran OS = Homo sapiens | 24 | kDa | 9 | 0 | 6 | 11 |
| OX = 9606 GN = RAN PE = 1 SV = 3 | | | | | | |
| Heterochromatin protein 1-binding protein 3 OS = Homo | 61 | kDa | 9 | 24 | 4 | 7 |
| sapiens OX = 9606 GN = HP1BP3 PE = 1 SV = 1 | | | | | | |
| Isoform 1 of Voltage-dependent anion-selective channel | 33 | kDa | 9 | 19 | 1 | 5 |
| protein 2 OS = Homo sapiens OX = 9606 GN = VDAC2 | | | | | | |
| Isoform 2 of Eukaryotic translation initiation factor 3 | 163 | kDa | 9 | 10 | 0 | 11 |
| subunit A OS = Homo sapiens OX = 9606 GN = EIF3A | | | | | | |
| Isoform 2 of Glutamine--tRNA ligase OS = Homo sapiens | 87 | kDa | 9 | 16 | 0 | 4 |
| OX = 9606 GN = QARS | | | | | | |
| Isoform 2 of Tropomyosin alpha-3 chain OS = Homo | 29 | kDa | 9 | 99 | 16 | 17 |
| sapiens OX = 9606 GN = TPM3 | | | | | | |
| Isoform 3 of Protein AHNAK2 OS = Homo sapiens | 606 | kDa | 9 | 0 | 0 | 1 |
| OX = 9606 GN = AHNAK2 | | | | | | |
| Isoform D of Eukaryotic translation initiation factor 4 | 159 | kDa | 9 | 7 | 0 | 0 |
| gamma 1 OS = Homo sapiens OX = 9606 GN = EIF4G1 | | | | | | |
| KH domain-containing, RNA-binding, signal | 48 | kDa | 9 | 24 | 5 | 9 |
| transduction-associated protein 1 OS = Homo sapiens | | | | | | |
| OX = 9606 GN = KHDRBS1 PE = 1 SV = 1 | | | | | | |
| Methionine--tRNA ligase, cytoplasmic OS = Homo | 101 | kDa | 9 | 1 | 0 | 0 |
| sapiens OX = 9606 GN = MARS PE = 1 SV = 2 | | | | | | |
| Myeloid-associated differentiation marker OS = Homo | 35 | kDa | 9 | 0 | 16 | 12 |
| sapiens OX = 9606 GN = MYADM PE = 1 SV = 2 | | | | | | |
| Neutral alpha-glucosidase AB OS = Homo sapiens | 107 | kDa | 9 | 21 | 0 | 13 |
| OX = 9606 GN = GANAB PE = 1 SV = 3 | | | | | | |
| Peroxiredoxin-5, mitochondrial OS = Homo sapiens | 22 | kDa | 9 | 6 | 0 | 5 |
| OX = 9606 GN = PRDX5 PE = 1 SV = 4 | | | | | | |
| Serine--tRNA ligase, cytoplasmic OS = Homo sapiens | 59 | kDa | 9 | 0 | 0 | 9 |
| OX = 9606 GN = SARS PE = 1 SV = 3 | | | | | | |
| Tryptophan--tRNA ligase, cytoplasmic OS = Homo | 53 | kDa | 9 | 6 | 1 | 7 |
| sapiens OX = 9606 GN = WARS PE = 1 SV = 2 | | | | | | |
| Vacuolar protein sorting-associated protein 35 OS = Homo | 92 | kDa | 9 | 5 | 0 | 4 |
| sapiens OX = 9606 GN = VPS35 PE = 1 SV = 2 | | | | | | |
| 26S proteasome non-ATPase regulatory subunit 3 | 61 | kDa | 8 | 21 | 0 | 6 |
| OS = Homo sapiens OX = 9606 GN = PSMD3 PE = 1 SV = 2 | | | | | | |
| 40S ribosomal protein S16 OS = Homo sapiens OX = 9606 | 16 | kDa | 8 | 0 | 9 | 7 |
| GN = RPS16 PE = 1 SV = 2 | | | | | | |
| 40S ribosomal protein S6 OS = Homo sapiens OX = 9606 | 29 | kDa | 8 | 0 | 5 | 11 |
| GN = RPS6 PE = 1 SV = 1 | | | | | | |
| 40S ribosomal protein S9 OS = Homo sapiens OX = 9606 | 23 | kDa | 8 | 54 | 11 | 10 |
| GN = RPS9 PE = 1 SV = 3 | | | | | | |
| ADP/ATP translocase 1 OS = Homo sapiens OX = 9606 | 33 | kDa | 8 | 0 | 0 | 12 |
| GN = SLC25A4 PE = 1 SV = 4 | | | | | | |
| Annexin A1 OS = Homo sapiens OX = 9606 GN = ANXA1 | 39 | kDa | 8 | 0 | 0 | 8 |
| PE = 1 SV = 2 | | | | | | |
| ATP-dependent RNA helicase DDX1 OS = Homo sapiens | 82 | kDa | 8 | 15 | 2 | 10 |
| OX = 9606 GN = DDX1 PE = 1 SV = 2 | | | | | | |
| Coatomer subunit gamma-1 OS = Homo sapiens | 98 | kDa | 8 | 9 | 1 | 6 |
| OX = 9606 GN = COPG1 PE = 1 SV = 1 | | | | | | |
| Elongation factor Tu, mitochondrial OS = Homo sapiens | 50 | kDa | 8 | 0 | 0 | 0 |
| OX = 9606 GN = TUFM PE = 1 SV = 2 | | | | | | |
| Erythrocyte band 7 integral membrane protein OS = Homo | 32 | kDa | 8 | 6 | 2 | 3 |
| sapiens OX = 9606 GN = STOM PE = 1 SV = 3 | | | | | | |
| Eukaryotic translation initiation factor 2 subunit 3 | 51 | kDa | 8 | 0 | 3 | 12 |
| OS = Homo sapiens OX = 9606 GN = EIF2S3 PE = 1 SV = 3 | | | | | | |
| Flotillin-1 OS = Homo sapiens OX = 9606 GN = FLOT1 | 47 | kDa | 8 | 25 | 1 | 4 |
| PE = 1 SV = 3 | | | | | | |
| Heterogeneous nuclear ribonucleoprotein L OS = Homo | 64 | kDa | 8 | 11 | 0 | 5 |
| sapiens OX = 9606 GN = HNRNPL PE = 1 SV = 2 | | | | | | |
| Isoform 2 of Ankycorbin OS = Homo sapiens OX = 9606 | 110 | kDa | 8 | 38 | 2 | 5 |
| GN = RAI14 | | | | | | |
| Isoform 2 of Bifunctional purine biosynthesis protein | 65 | kDa | 8 | 5 | 0 | 4 |
| PURH OS = Homo sapiens OX = 9606 GN = ATIC | | | | | | |
| Isoform 2 of Eukaryotic translation initiation factor 5A-1 | 20 | kDa | 8 | 14 | 1 | 7 |
| OS = Homo sapiens OX = 9606 GN = EIF5A | | | | | | |
| Isoform 2 of Golgi apparatus protein 1 OS = Homo | 137 | kDa | 8 | 4 | 9 | 1 |
| sapiens OX = 9606 GN = GLG1 | | | | | | |
| Isoform 2 of Poly(rC)-binding protein 2 OS = Homo | 39 | kDa | 8 | 4 | 0 | 4 |
| sapiens OX = 9606 GN = PCBP2 | | | | | | |
| Isoform 3 of Heterogeneous nuclear ribonucleoprotein | 33 | kDa | 8 | 20 | 6 | 11 |
| DO OS = Homo sapiens OX = 9606 GN = HNRNPD | | | | | | |
| Isoform B of Phosphate carrier protein, mitochondrial | 40 | kDa | 8 | 7 | 5 | 8 |
| OS = Homo sapiens OX = 9606 GN = SLC25A3 | | | | | | |
| Kinesin-1 heavy chain OS = Homo sapiens OX = 9606 | 110 | kDa | 8 | 5 | 0 | 3 |
| GN = KIF5B PE = 1 SV = 1 | | | | | | |
| Malate dehydrogenase, mitochondrial OS = Homo sapiens | 36 | kDa | 8 | 17 | 0 | 13 |
| OX = 9606 GN = MDH2 PE = 1 SV = 3 | | | | | | |
| Mitochondrial carrier homolog 2 OS = Homo sapiens | 33 | kDa | 8 | 0 | 2 | 4 |
| OX = 9606 GN = MTCH2 PE = 1 SV = 1 | | | | | | |
| Pre-mRNA-processing factor 19 OS = Homo sapiens | 55 | kDa | 8 | 0 | 0 | 14 |
| OX = 9606 GN = PRPF19 PE = 1 SV = 1 | | | | | | |
| Prohibitin OS = Homo sapiens OX = 9606 GN = PHB PE = 1 | 30 | kDa | 8 | 14 | 2 | 15 |
| SV = 1 | | | | | | |
| Protein transport protein Sec23A OS = Homo sapiens | 86 | kDa | 8 | 9 | 1 | 10 |
| OX = 9606 GN = SEC23A PE = 1 SV = 2 | | | | | | |
| Protein transport protein Sec61 subunit alpha isoform 1 | 52 | kDa | 8 | 2 | 1 | 5 |
| OS = Homo sapiens OX = 9606 GN = SEC61A1 PE = 1 SV = 2 | | | | | | |
| Septin-11 OS = Homo sapiens OX = 9606 GN = SEPT11 | 49 | kDa | 8 | 27 | 0 | 9 |
| PE = 1 SV = 3 | | | | | | |
| Small nuclear ribonucleoprotein Sm D1 OS = Homo | 13 | kDa | 8 | 0 | 6 | 7 |
| sapiens OX = 9606 GN = SNRPD1 PE = 1 SV = 1 | | | | | | |
| SWISS-PROT: P15497 (Bos taurus) Apolipoprotein A-I | 30 | kDa | 8 | 21 | 6 | 4 |
| precursor | | | | | | |
| THO complex subunit 4 OS = Homo sapiens OX = 9606 | 27 | kDa | 8 | 37 | 7 | 11 |
| GN = ALYREF PE = 1 SV = 3 | | | | | | |
| TREMBL: Q3ZBS7 (Bos taurus) Vitronectin | 54 | kDa | 8 | 26 | 12 | 11 |
| Ubiquitin-40S ribosomal protein S27a OS = Homo sapiens | 18 | kDa | 8 | 0 | 13 | 10 |
| OX = 9606 GN = RPS27A PE = 1 SV = 2 | | | | | | |
| 26S proteasome non-ATPase regulatory subunit 12 | 53 | kDa | 7 | 6 | 0 | 2 |
| OS = Homo sapiens OX = 9606 GN = PSMD12 PE = 1 SV = 3 | | | | | | |
| 26S proteasome regulatory subunit 8 OS = Homo sapiens | 46 | kDa | 7 | 6 | 0 | 12 |
| OX = 9606 GN = PSMC5 PE = 1 SV = 1 | | | | | | |
| 40S ribosomal protein S14 OS = Homo sapiens OX = 9606 | 16 | kDa | 7 | 40 | 13 | 9 |
| GN = RPS14 PE = 1 SV = 3 | | | | | | |
| 40S ribosomal protein S19 OS = Homo sapiens OX = 9606 | 16 | kDa | 7 | 0 | 12 | 15 |
| GN = RPS19 PE = 1 SV = 2 | | | | | | |
| 40S ribosomal protein S23 OS = Homo sapiens OX = 9606 | 16 | kDa | 7 | 0 | 4 | 12 |
| GN = RPS23 PE = 1 SV = 3 | | | | | | |
| 60S ribosomal protein L30 OS = Homo sapiens OX = 9606 | 13 | kDa | 7 | 0 | 8 | 8 |
| GN = RPL30 PE = 1 SV = 2 | | | | | | |
| ADP-ribosylation factor 3 OS = Homo sapiens OX = 9606 | 21 | kDa | 7 | 11 | 7 | 7 |
| GN = ARF3 PE = 1 SV = 2 | | | | | | |
| Aminoacyl tRNA synthase complex-interacting | 35 | kDa | 7 | 0 | 0 | 6 |
| multifunctional protein 2 OS = Homo sapiens OX = 9606 | | | | | | |
| GN = AIMP2 PE = 1 SV = 2 | | | | | | |
| CAD protein OS = Homo sapiens OX = 9606 GN = CAD | 243 | kDa | 7 | 0 | 0 | 0 |
| PE = 1 SV = 3 | | | | | | |
| Catenin beta-1 OS = Homo sapiens OX = 9606 | 85 | kDa | 7 | 3 | 0 | 6 |
| GN = CTNNB1 PE = 1 SV = 1 | | | | | | |
| Cell division control protein 42 homolog OS = Homo | 21 | kDa | 7 | 0 | 2 | 11 |
| sapiens OX = 9606 GN = CDC42 PE = 1 SV = 2 | | | | | | |
| Chloride intracellular channel protein 1 OS = Homo | 27 | kDa | 7 | 0 | 0 | 5 |
| sapiens OX = 9606 GN = CLIC1 PE = 1 SV = 4 | | | | | | |
| Coatomer subunit beta OS = Homo sapiens OX = 9606 | 107 | kDa | 7 | 5 | 0 | 1 |
| GN = COPB1 PE = 1 SV = 3 | | | | | | |
| Eukaryotic initiation factor 4A-III OS = Homo sapiens | 47 | kDa | 7 | 20 | 4 | 8 |
| OX = 9606 GN = EIF4A3 PE = 1 SV = 4 | | | | | | |
| Heterogeneous nuclear ribonucleoprotein H OS = Homo | 51 | kDa | 7 | 23 | 1 | 9 |
| sapiens OX = 9606 GN = HNRNPH1 PE = 1 SV = 1 | | | | | | |
| Importin-5 OS = Homo sapiens OX = 9606 GN = IPO5 | 124 | kDa | 7 | 5 | 0 | 3 |
| PE = 1 SV = 4 | | | | | | |
| Inhibitor of nuclear factor kappa-B kinase-interacting | 39 | kDa | 7 | 7 | 6 | 7 |
| protein OS = Homo sapiens OX = 9606 GN = IKBIP PE = 1 | | | | | | |
| SV = 1 | | | | | | |
| Isoform 2 of 26S proteasome non-ATPase regulatory | 48 | kDa | 7 | 12 | 0 | 5 |
| subunit 11 OS = Homo sapiens OX = 9606 GN = PSMD11 | | | | | | |
| Isoform 2 of F-actin-capping protein subunit beta | 31 | kDa | 7 | 46 | 10 | 5 |
| OS = Homo sapiens OX = 9606 GN = CAPZB | | | | | | |
| Isoform 2 of Heterogeneous nuclear ribonucleoprotein D- | 34 | kDa | 7 | 8 | 0 | 7 |
| like OS = Homo sapiens OX = 9606 GN = HNRNPDL | | | | | | |
| Isoform 2 of Integrin alpha-3 OS = Homo sapiens | 119 | kDa | 7 | 2 | 0 | 5 |
| OX = 9606 GN = ITGA3 | | | | | | |
| Isoform 2 of Nucleolar RNA helicase 2 OS = Homo | 80 | kDa | 7 | 16 | 2 | 7 |
| sapiens OX = 9606 GN = DDX21 | | | | | | |
| Isoform 2 of PDZ and LIM domain protein 7 OS = Homo | 47 | kDa | 7 | 13 | 0 | 5 |
| sapiens OX = 9606 GN = PDLIM7 | | | | | | |
| Isoform 2 of Spermine synthase OS = Homo sapiens | 35 | kDa | 7 | 2 | 0 | 6 |
| OX = 9606 GN = SMS | | | | | | |
| Isoform 2 of Transketolase OS = Homo sapiens OX = 9606 | 69 | kDa | 7 | 29 | 0 | 9 |
| GN = TKT | | | | | | |
| Isoform 3 of 116 kDa U5 small nuclear ribonucleoprotein | 108 | kDa | 7 | 6 | 0 | 9 |
| component OS = Homo sapiens OX = 9606 GN = EFTUD2 | | | | | | |
| Isoform 3 of 60S ribosomal protein L17 OS = Homo | 26 | kDa | 7 | 54 | 5 | 8 |
| sapiens OX = 9606 GN = RPL17 | | | | | | |
| Isoform 3 of Integrin alpha-V OS = Homo sapiens | 111 | kDa | 7 | 22 | 2 | 12 |
| OX = 9606 GN = ITGAV | | | | | | |
| Isoform SV3 of Supervillin OS = Homo sapiens OX = 9606 | 245 | kDa | 7 | 46 | 0 | 1 |
| GN = SVIL | | | | | | |
| NADH dehydrogenase [ubiquinone] 1 alpha subcomplex | 43 | kDa | 7 | 12 | 1 | 7 |
| subunit 9, mitochondrial OS = Homo sapiens OX = 9606 | | | | | | |
| GN = NDUFA9 PE = 1 SV = 2 | | | | | | |
| Peroxiredoxin-1 OS = Homo sapiens OX = 9606 | 22 | kDa | 7 | 0 | 0 | 7 |
| GN = PRDX1 PE = 1 SV = 1 | | | | | | |
| Poly(rC)-binding protein 1 OS = Homo sapiens OX = 9606 | 37 | kDa | 7 | 0 | 0 | 2 |
| GN = PCBP1 PE = 1 SV = 2 | | | | | | |
| Pre-mRNA-processing-splicing factor 8 OS = Homo | 274 | kDa | 7 | 6 | 1 | 3 |
| sapiens OX = 9606 GN = PRPF8 PE = 1 SV = 2 | | | | | | |
| Probable ATP-dependent RNA helicase DDX5 | 69 | kDa | 7 | 25 | 7 | 19 |
| OS = Homo sapiens OX = 9606 GN = DDX5 PE = 1 SV = 1 | | | | | | |
| Prohibitin-2 OS = Homo sapiens OX = 9606 GN = PHB2 | 33 | kDa | 7 | 25 | 5 | 12 |
| PE = 1 SV = 2 | | | | | | |
| Protein disulfide-isomerase A4 OS = Homo sapiens | 73 | kDa | 7 | 4 | 0 | 4 |
| OX = 9606 GN = PDIA4 PE = 1 SV = 2 | | | | | | |
| Ras GTPase-activating protein-binding protein 1 | 52 | kDa | 7 | 19 | 2 | 8 |
| OS = Homo sapiens OX = 9606 GN = G3BP1 PE = 1 SV = 1 | | | | | | |
| Ras-related protein Rab-1B OS = Homo sapiens OX = 9606 | 22 | kDa | 7 | 0 | 0 | 8 |
| GN = RAB1B PE = 1 SV = 1 | | | | | | |
| RNA-binding motif protein, X chromosome OS = Homo | 42 | kDa | 7 | 22 | 5 | 13 |
| sapiens OX = 9606 GN = RBMX PE = 1 SV = 3 | | | | | | |
| Splicing factor 3B subunit 1 OS = Homo sapiens | 146 | kDa | 7 | 16 | 1 | 3 |
| OX = 9606 GN = SF3B1 PE = 1 SV = 3 | | | | | | |
| SWISS-PROT: P02535-1 Tax_Id = 10090 | 58 | kDa | 7 | 39 | 7 | 0 |
| Gene_Symbol = Krt10 Isoform 1 of Keratin, type I | | | | | | |
| cytoskeletal 10 | | | | | | |
| SWISS-PROT: P08730-1 Tax_Id = 10090 | 48 | kDa | 7 | 451 | 17 | 31 |
| Gene_Symbol = Krt13 Isoform 1 of Keratin, type I | | | | | | |
| cytoskeletal 13 | | | | | | |
| SWISS-PROT: Q6IFZ6 Tax_Id = 10090 | 61 | kDa | 7 | 32 | 10 | 5 |
| Gene_Symbol = Krt77 Keratin, type II cytoskeletal 1b | | | | | | |
| Talin-2 OS = Homo sapiens OX = 9606 GN = TLN2 PE = 1 | 272 | kDa | 7 | 0 | 0 | 1 |
| SV = 4 | | | | | | |
| Transcription intermediary factor 1-beta OS = Homo | 89 | kDa | 7 | 5 | 0 | 0 |
| sapiens OX = 9606 GN = TRIM28 PE = 1 SV = 5 | | | | | | |
| TREMBL: Q9TRI1 (Bos taurus) similar to inter-alpha- | 106 | kDa | 7 | 57 | 7 | 8 |
| trypsin inhibitor heavy chain2 | | | | | | |
| U5 small nuclear ribonucleoprotein 200 kDa helicase | 245 | kDa | 7 | 1 | 0 | 0 |
| OS = Homo sapiens OX = 9606 GN = SNRNP200 PE = 1 | | | | | | |
| SV = 2 | | | | | | |
| Valine--tRNA ligase OS = Homo sapiens OX = 9606 | 140 | kDa | 7 | 8 | 0 | 4 |
| GN = VARS PE = 1 SV = 4 | | | | | | |
| 26S proteasome regulatory subunit 4 OS = Homo sapiens | 49 | kDa | 6 | 25 | 1 | 6 |
| OX = 9606 GN = PSMC1 PE = 1 SV = 1 | | | | | | |
| 40S ribosomal protein S26 OS = Homo sapiens OX = 9606 | 13 | kDa | 6 | 0 | 6 | 6 |
| GN = RPS26 PE = 1 SV = 3 | | | | | | |
| 60S ribosomal protein L24 OS = Homo sapiens OX = 9606 | 18 | kDa | 6 | 0 | 5 | 6 |
| GN = RPL24 PE = 1 SV = 1 | | | | | | |
| 60S ribosomal protein L27a OS = Homo sapiens | 17 | kDa | 6 | 0 | 6 | 4 |
| OX = 9606 GN = RPL27A PE = 1 SV = 2 | | | | | | |
| Alpha-centractin OS = Homo sapiens OX = 9606 | 43 | kDa | 6 | 26 | 3 | 11 |
| GN = ACTR1A PE = 1 SV = 1 | | | | | | |
| ATP-dependent RNA helicase DDX18 OS = Homo | 75 | kDa | 6 | 5 | 3 | 8 |
| sapiens OX = 9606 GN = DDX18 PE = 1 SV = 2 | | | | | | |
| Collagen alpha-1(IV) chain OS = Homo sapiens OX = 9606 | 161 | kDa | 6 | 69 | 21 | 7 |
| GN = COL4A1 PE = 1 SV = 4 | | | | | | |
| Collagen alpha-1(VIII) chain OS = Homo sapiens | 73 | kDa | 6 | 0 | 29 | 22 |
| OX = 9606 GN = COL8A1 PE = 1 SV = 2 | | | | | | |
| Cullin-associated NEDD8-dissociated protein 1 | 136 | kDa | 6 | 0 | 0 | 3 |
| OS = Homo sapiens GN = CAND1 PE = 1 SV = 2 | | | | | | |
| Dihydrolipoyllysine-residue succinyltransferase | 49 | kDa | 6 | 17 | 0 | 8 |
| component of 2-oxoglutarate dehydrogenase complex, | | | | | | |
| mitochondrial OS = Homo sapiens OX = 9606 GN = DLST | | | | | | |
| PE = 1 SV = 4 | | | | | | |
| Dihydropyrimidinase-related protein 2 OS = Homo | 62 | kDa | 6 | 3 | 0 | 5 |
| sapiens OX = 9606 GN = DPYSL2 PE = 1 SV = 1 | | | | | | |
| Eukaryotic translation initiation factor 3 subunit M | 43 | kDa | 6 | 11 | 0 | 5 |
| OS = Homo sapiens OX = 9606 GN = EIF3M PE = 1 SV = 1 | | | | | | |
| Exportin-1 OS = Homo sapiens OX = 9606 GN = XPO1 | 123 | kDa | 6 | 0 | 0 | 0 |
| PE = 1 SV = 1 | | | | | | |
| F-actin-capping protein subunit alpha-2 OS = Homo | 33 | kDa | 6 | 33 | 8 | 9 |
| sapiens OX = 9606 GN = CAPZA2 PE = 1 SV = 3 | | | | | | |
| Glycogen phosphorylase, brain form OS = Homo sapiens | 97 | kDa | 6 | 0 | 0 | 1 |
| OX = 9606 GN = PYGB PE = 1 SV = 5 | | | | | | |
| Importin-7 OS = Homo sapiens OX = 9606 GN = IPO7 | 120 | kDa | 6 | 0 | 0 | 0 |
| PE = 1 SV = 1 | | | | | | |
| Isoform 2 of 6-phosphogluconate dehydrogenase, | 52 | kDa | 6 | 4 | 0 | 3 |
| decarboxylating OS = Homo sapiens OX = 9606 GN = PGD | | | | | | |
| Isoform 2 of Coatomer subunit beta′ OS = Homo sapiens | 99 | kDa | 6 | 10 | 0 | 4 |
| OX = 9606 GN = COPB2 | | | | | | |
| Isoform 2 of Coronin-1C OS = Homo sapiens OX = 9606 | 54 | kDa | 6 | 36 | 5 | 7 |
| GN = CORO1C | | | | | | |
| Isoform 2 of Elongation factor 1-delta OS = Homo sapiens | 71 | kDa | 6 | 32 | 3 | 4 |
| OX = 9606 GN = EEF1D | | | | | | |
| Isoform 2 of Inverted formin-2 OS = Homo sapiens | 135 | kDa | 6 | 4 | 0 | 1 |
| OX = 9606 GN = INF2 | | | | | | |
| Isoform 2 of Programmed cell death 6-interacting protein | 97 | kDa | 6 | 3 | 0 | 4 |
| OS = Homo sapiens OX = 9606 GN = PDCD6IP | | | | | | |
| Isoform 2 of Surfeit locus protein 4 OS = Homo sapiens | 18 | kDa | 6 | 10 | 3 | 7 |
| OX = 9606 GN = SURF4 | | | | | | |
| Isoform 3 of Plasminogen activator inhibitor 1 RNA- | 43 | kDa | 6 | 11 | 6 | 7 |
| binding protein OS = Homo sapiens OX = 9606 | | | | | | |
| GN = SERBP1 | | | | | | |
| Isoform 5 of Phosphatidylinositol-binding clathrin | 70 | kDa | 6 | 5 | 0 | 7 |
| assembly protein OS = Homo sapiens GN = PICALM | | | | | | |
| Junction plakoglobin OS = Homo sapiens OX = 9606 | 82 | kDa | 6 | 38 | 0 | 6 |
| GN = JUP PE = 1 SV = 3 | | | | | | |
| Lamina-associated polypeptide 2, isoforms beta/gamma | 51 | kDa | 6 | 26 | 7 | 7 |
| OS = Homo sapiens OX = 9606 GN = TMPO PE = 1 SV = 2 | | | | | | |
| Leucine-rich repeat-containing protein 59 OS = Homo | 35 | kDa | 6 | 0 | 7 | 10 |
| sapiens OX = 9606 GN = LRRC59 PE = 1 SV = 1 | | | | | | |
| Multifunctional protein ADE2 OS = Homo sapiens | 47 | kDa | 6 | 3 | 0 | 2 |
| OX = 9606 GN = PAICS PE = 1 SV = 3 | | | | | | |
| Nicotinamide N-methyltransferase OS = Homo sapiens | 30 | kDa | 6 | 0 | 0 | 1 |
| OX = 9606 GN = NNMT PE = 1 SV = 1 | | | | | | |
| Poly [ADP-ribose] polymerase 4 OS = Homo sapiens | 193 | kDa | 6 | 19 | 0 | 5 |
| OX = 9606 GN = PARP4 PE = 1 SV = 3 | | | | | | |
| Protein S100-A6 OS = Homo sapiens OX = 9606 | 10 | kDa | 6 | 0 | 1 | 4 |
| GN = S100A6 PE = 1 SV = 1 | | | | | | |
| RuvB-like 2 OS = Homo sapiens OX = 9606 GN = RUVBL2 | 51 | kDa | 6 | 19 | 0 | 5 |
| PE = 1 SV = 3 | | | | | | |
| Sarcoplasmic/endoplasmic reticulum calcium ATPase 2 | 115 | kDa | 6 | 6 | 0 | 0 |
| OS = Homo sapiens GN = ATP2A2 PE = 1 SV = 1 | | | | | | |
| Signal transducer and activator of transcription 1- | 87 | kDa | 6 | 15 | 1 | 5 |
| alpha/beta OS = Homo sapiens OX = 9606 GN = STAT1 | | | | | | |
| PE = 1 SV = 2 | | | | | | |
| Synaptic vesicle membrane protein VAT-1 homolog | 42 | kDa | 6 | 6 | 0 | 7 |
| OS = Homo sapiens OX = 9606 GN = VAT1 PE = 1 SV = 2 | | | | | | |
| Ubiquitin carboxyl-terminal hydrolase OS = Homo sapiens | 27 | kDa | 6 | 0 | 0 | 4 |
| OX = 9606 GN = UCHL1 PE = 1 SV = 1 | | | | | | |
| Very-long-chain 3-oxoacyl-CoA reductase OS = Homo | 34 | kDa | 6 | 0 | 2 | 6 |
| sapiens OX = 9606 GN = HSD17B12 PE = 1 SV = 2 | | | | | | |
| 26S proteasome non-ATPase regulatory subunit 13 | 43 | kDa | 5 | 9 | 0 | 4 |
| OS = Homo sapiens OX = 9606 GN = PSMD13 PE = 1 SV = 2 | | | | | | |
| 26S proteasome regulatory subunit 10B OS = Homo | 44 | kDa | 5 | 0 | 0 | 5 |
| sapiens OX = 9606 GN = PSMC6 PE = 1 SV = 1 | | | | | | |
| 40S ribosomal protein S10 OS = Homo sapiens OX = 9606 | 19 | kDa | 5 | 38 | 13 | 12 |
| GN = RPS10 PE = 1 SV = 1 | | | | | | |
| 40S ribosomal protein S11 OS = Homo sapiens OX = 9606 | 18 | kDa | 5 | 26 | 3 | 7 |
| GN = RPS11 PE = 1 SV = 3 | | | | | | |
| 60S ribosomal protein L21 OS = Homo sapiens OX = 9606 | 19 | kDa | 5 | 34 | 8 | 7 |
| GN = RPL21 PE = 1 SV = 2 | | | | | | |
| 60S ribosomal protein L22 OS = Homo sapiens OX = 9606 | 15 | kDa | 5 | 0 | 6 | 8 |
| GN = RPL22 PE = 1 SV = 2 | | | | | | |
| 60S ribosomal protein L23a (Fragment) OS = Homo | 19 | kDa | 5 | 0 | 9 | 9 |
| sapiens OX = 9606 GN = RPL23A PE = 1 SV = 1 | | | | | | |
| 60S ribosomal protein L27 OS = Homo sapiens OX = 9606 | 16 | kDa | 5 | 0 | 6 | 11 |
| GN = RPL27 PE = 1 SV = 2 | | | | | | |
| 60S ribosomal protein L28 OS = Homo sapiens OX = 9606 | 16 | kDa | 5 | 13 | 4 | 4 |
| GN = RPL28 PE = 1 SV = 3 | | | | | | |
| 60S ribosomal protein L31 OS = Homo sapiens OX = 9606 | 14 | kDa | 5 | 33 | 8 | 6 |
| GN = RPL31 PE = 1 SV = 1 | | | | | | |
| 60S ribosomal protein L36 OS = Homo sapiens OX = 9606 | 12 | kDa | 5 | 0 | 7 | 7 |
| GN = RPL36 PE = 1 SV = 3 | | | | | | |
| Actin-related protein 2/3 complex subunit 3 OS = Homo | 21 | kDa | 5 | 0 | 4 | 7 |
| sapiens OX = 9606 GN = ARPC3 PE = 1 SV = 3 | | | | | | |
| Actin-related protein 2/3 complex subunit 4 OS = Homo | 20 | kDa | 5 | 19 | 2 | 6 |
| sapiens OX = 9606 GN = ARPC4 PE = 1 SV = 3 | | | | | | |
| Calpain-1 catalytic subunit OS = Homo sapiens OX = 9606 | 82 | kDa | 5 | 27 | 0 | 6 |
| GN = CAPN1 PE = 1 SV = 1 | | | | | | |
| Calponin-2 OS = Homo sapiens OX = 9606 GN = CNN2 | 34 | kDa | 5 | 5 | 1 | 6 |
| PE = 1 SV = 4 | | | | | | |
| Cathepsin D OS = Homo sapiens OX = 9606 GN = CTSD | 45 | kDa | 5 | 0 | 0 | 3 |
| PE = 1 SV = 1 | | | | | | |
| Cleavage and polyadenylation specificity factor subunit 5 | 26 | kDa | 5 | 0 | 0 | 6 |
| OS = Homo sapiens OX = 9606 GN = NUDT21 PE = 1 SV = 1 | | | | | | |
| Coatomer subunit epsilon OS = Homo sapiens GN = COPE | 34 | kDa | 5 | 14 | 2 | 8 |
| PE = 1 SV = 3 | | | | | | |
| Copine-3 OS = Homo sapiens OX = 9606 GN = CPNE3 | 60 | kDa | 5 | 0 | 0 | 1 |
| PE = 1 SV = 1 | | | | | | |
| Cytochrome c oxidase subunit 2 OS = Homo sapiens | 26 | kDa | 5 | 0 | 0 | 6 |
| OX = 9606 GN = MT-CO2 PE = 1 SV = 1 | | | | | | |
| DNA-(apurinic or apyrimidinic site) lyase OS = Homo | 36 | kDa | 5 | 8 | 0 | 3 |
| sapiens OX = 9606 GN = APEX1 PE = 1 SV = 2 | | | | | | |
| Erlin-1 OS = Homo sapiens OX = 9606 GN = ERLIN1 PE = 1 | 39 | kDa | 5 | 0 | 0 | 4 |
| SV = 1 | | | | | | |
| Eukaryotic translation elongation factor 1 epsilon-1 | 20 | kDa | 5 | 7 | 2 | 7 |
| OS = Homo sapiens GN = EEF1E1 PE = 1 SV = 1 | | | | | | |
| Eukaryotic translation initiation factor 2 subunit 1 | 36 | kDa | 5 | 9 | 1 | 7 |
| OS = Homo sapiens OX = 9606 GN = EIF2S1 PE = 1 SV = 3 | | | | | | |
| Eukaryotic translation initiation factor 3 subunit H | 42 | kDa | 5 | 0 | 0 | 3 |
| OS = Homo sapiens OX = 9606 GN = EIF3H PE = 1 SV = 1 | | | | | | |
| Heat shock 70 kDa protein 4 OS = Homo sapiens | 94 | kDa | 5 | 4 | 0 | 1 |
| OX = 9606 GN = HSPA4 PE = 1 SV = 4 | | | | | | |
| High mobility group protein B1 OS = Homo sapiens | 25 | kDa | 5 | 0 | 0 | 3 |
| OX = 9606 GN = HMGB1 PE = 1 SV = 3 | | | | | | |
| Importin-9 OS = Homo sapiens OX = 9606 GN = IPO9 | 116 | kDa | 5 | 0 | 0 | 4 |
| PE = 1 SV = 3 | | | | | | |
| Isoform 2 of 26S proteasome non-ATPase regulatory | 102 | kDa | 5 | 10 | 0 | 3 |
| subunit 1 OS = Homo sapiens OX = 9606 GN = PSMD1 | | | | | | |
| Isoform 2 of Calcium-binding mitochondrial carrier | 74 | kDa | 5 | 10 | 0 | 9 |
| protein Aralar2 OS = Homo sapiens OX = 9606 | | | | | | |
| GN = SLC25A13 | | | | | | |
| Isoform 2 of Collagen alpha-3(VI) chain OS = Homo | 321 | kDa | 5 | 0 | 1 | 1 |
| sapiens OX = 9606 GN = COL6A3 | | | | | | |
| Isoform 2 of Eukaryotic translation initiation factor 3 | 99 | kDa | 5 | 5 | 0 | 6 |
| subunit B OS = Homo sapiens OX = 9606 GN = EIF3B | | | | | | |
| Isoform 2 of Glucose-6-phosphate isomerase OS = Homo | 64 | kDa | 5 | 4 | 0 | 2 |
| sapiens OX = 9606 GN = GPI | | | | | | |
| Isoform 2 of HLA class I histocompatibility antigen, A- | 41 | kDa | 5 | 3 | 0 | 3 |
| 11 alpha chain OS = Homo sapiens OX = 9606 GN = HLA- | | | | | | |
| A | | | | | | |
| Isoform 2 of Myb-binding protein 1A OS = Homo sapiens | 149 | kDa | 5 | 1 | 0 | 0 |
| OX = 9606 GN = MYBBP1A | | | | | | |
| Isoform 2 of Serine/arginine-rich splicing factor 2 | 24 | kDa | 5 | 2 | 0 | 2 |
| OS = Homo sapiens OX = 9606 GN = SRSF2 | | | | | | |
| Isoform 2 of U1 small nuclear ribonucleoprotein 70 kDa | 51 | kDa | 5 | 0 | 0 | 5 |
| OS = Homo sapiens OX = 9606 GN = SNRNP70 | | | | | | |
| Isoform 3 of Glutaminase kidney isoform, mitochondrial | 65 | kDa | 5 | 7 | 0 | 3 |
| OS = Homo sapiens OX = 9606 GN = GLS | | | | | | |
| Isoform 4 of AP-3 complex subunit delta-1 OS = Homo | 115 | kDa | 5 | 5 | 0 | 2 |
| sapiens GN = AP3D1 | | | | | | |
| Isoform 4 of Protein phosphatase 1 regulatory subunit | 109 | kDa | 5 | 10 | 3 | 7 |
| 12A OS = Homo sapiens OX = 9606 GN = PPP1R12A | | | | | | |
| Isoform Short of Eukaryotic translation initiation factor | 25 | kDa | 5 | 5 | 1 | 4 |
| 4H OS = Homo sapiens OX = 9606 GN = EIF4H | | | | | | |
| Lon protease homolog, mitochondrial OS = Homo sapiens | 106 | kDa | 5 | 0 | 0 | 6 |
| GN = LONP1 PE = 1 SV = 2 | | | | | | |
| Mannosyl-oligosaccharide glucosidase OS = Homo | 92 | kDa | 5 | 22 | 4 | 11 |
| sapiens GN = MOGS PE = 1 SV = 5 | | | | | | |
| Prolyl 3-hydroxylase 1 OS = Homo sapiens OX = 9606 | 83 | kDa | 5 | 6 | 0 | 3 |
| GN = P3H1 PE = 1 SV = 2 | | | | | | |
| Proteasome subunit alpha type-4 OS = Homo sapiens | 29 | kDa | 5 | 0 | 0 | 7 |
| OX = 9606 GN = PSMA4 PE = 1 SV = 1 | | | | | | |
| Protein LYRIC OS = Homo sapiens OX = 9606 | 64 | kDa | 5 | 10 | 6 | 9 |
| GN = MTDH PE = 1 SV = 2 | | | | | | |
| Protein/nucleic acid deglycase DJ-1 OS = Homo sapiens | 20 | kDa | 5 | 0 | 0 | 3 |
| OX = 9606 GN = PARK7 PE = 1 SV = 2 | | | | | | |
| Ras-related protein Rab-1A OS = Homo sapiens OX = 9606 | 23 | kDa | 5 | 13 | 2 | 8 |
| GN = RAB1A PE = 1 SV = 3 | | | | | | |
| Ras-related protein Rab-3B OS = Homo sapiens OX = 9606 | 25 | kDa | 5 | 0 | 2 | 7 |
| GN = RAB3B PE = 1 SV = 2 | | | | | | |
| Rho GDP-dissociation inhibitor 1 OS = Homo sapiens | 23 | kDa | 5 | 0 | 0 | 4 |
| OX = 9606 GN = ARHGDIA PE = 1 SV = 3 | | | | | | |
| Signal recognition particle subunit SRP72 OS = Homo | 75 | kDa | 5 | 8 | 1 | 8 |
| sapiens OX = 9606 GN = SRP72 PE = 1 SV = 3 | | | | | | |
| Small nuclear ribonucleoprotein Sm D2 OS = Homo | 14 | kDa | 5 | 11 | 0 | 5 |
| sapiens OX = 9606 GN = SNRPD2 PE = 1 SV = 1 | | | | | | |
| Sorting and assembly machinery component 50 homolog | 52 | kDa | 5 | 19 | 4 | 12 |
| OS = Homo sapiens OX = 9606 GN = SAMM50 PE = 1 SV = 3 | | | | | | |
| Src substrate cortactin OS = Homo sapiens OX = 9606 | 62 | kDa | 5 | 38 | 12 | 8 |
| GN = CTTN PE = 1 SV = 2 | | | | | | |
| SWI/SNF complex subunit SMARCC1 OS = Homo | 123 | kDa | 5 | 0 | 0 | 2 |
| sapiens OX = 9606 GN = SMARCC1 PE = 1 SV = 3 | | | | | | |
| Transcription factor BTF3 OS = Homo sapiens OX = 9606 | 22 | kDa | 5 | 8 | 0 | 7 |
| GN = BTF3 PE = 1 SV = 1 | | | | | | |
| Translocon-associated protein subunit delta OS = Homo | 19 | kDa | 5 | 0 | 2 | 4 |
| sapiens OX = 9606 GN = SSR4 PE = 1 SV = 1 | | | | | | |
| TREMBL: Q1RMK2 (Bos taurus) IGHM protein | 65 | kDa | 5 | 0 | 0 | 0 |
| WD repeat-containing protein 1 OS = Homo sapiens | 66 | kDa | 5 | 11 | 0 | 5 |
| OX = 9606 GN = WDR1 PE = 1 SV = 4 | | | | | | |
| Zyxin OS = Homo sapiens OX = 9606 GN = ZYX PE = 1 | 61 | kDa | 5 | 13 | 0 | 4 |
| SV = 1 | | | | | | |
| 2-oxoglutarate dehydrogenase, mitochondrial OS = Homo | 116 | kDa | 4 | 2 | 0 | 6 |
| sapiens OX = 9606 GN = OGDH PE = 1 SV = 3 | | | | | | |
| 40S ribosomal protein S27-like OS = Homo sapiens | 9 | kDa | 4 | 0 | 1 | 1 |
| OX = 9606 GN = RPS27L PE = 1 SV = 3 | | | | | | |
| Adenosylhomocysteinase OS = Homo sapiens OX = 9606 | 48 | kDa | 4 | 4 | 0 | 2 |
| GN = AHCY PE = 1 SV = 4 | | | | | | |
| ADP-ribosylation factor 6 OS = Homo sapiens OX = 9606 | 20 | kDa | 4 | 0 | 0 | 2 |
| GN = ARF6 PE = 1 SV = 2 | | | | | | |
| Alanine--tRNA ligase, cytoplasmic OS = Homo sapiens | 107 | kDa | 4 | 0 | 0 | 2 |
| OX = 9606 GN = AARS PE = 1 SV = 2 | | | | | | |
| ATPase family AAA domain-containing protein 3A | 71 | kDa | 4 | 22 | 5 | 9 |
| OS = Homo sapiens OX = 9606 GN = ATAD3A PE = 1 SV = 2 | | | | | | |
| Coatomer subunit delta OS = Homo sapiens OX = 9606 | 57 | kDa | 4 | 10 | 0 | 5 |
| GN = ARCN1 PE = 1 SV = 1 | | | | | | |
| Cold-inducible RNA-binding protein OS = Homo sapiens | 19 | kDa | 4 | 3 | 0 | 4 |
| GN = CIRBP PE = 1 SV = 1 | | | | | | |
| Copine-1 OS = Homo sapiens OX = 9606 GN = CPNE1 | 59 | kDa | 4 | 0 | 0 | 2 |
| PE = 1 SV = 1 | | | | | | |
| Cytochrome b-c1 complex subunit 2, mitochondrial | 48 | kDa | 4 | 0 | 0 | 8 |
| OS = Homo sapiens OX = 9606 GN = UQCRC2 PE = 1 SV = 3 | | | | | | |
| DBH-like monooxygenase protein 1 OS = Homo sapiens | 70 | kDa | 4 | 17 | 5 | 4 |
| OX = 9606 GN = MOXD1 PE = 1 SV = 1 | | | | | | |
| EH domain-containing protein 1 OS = Homo sapiens | 61 | kDa | 4 | 11 | 0 | 9 |
| OX = 9606 GN = EHD1 PE = 1 SV = 2 | | | | | | |
| Eukaryotic translation initiation factor 3 subunit C | 105 | kDa | 4 | 4 | 0 | 5 |
| OS = Homo sapiens OX = 9606 GN = EIF3C PE = 1 SV = 1 | | | | | | |
| Eukaryotic translation initiation factor 4 gamma 2 | 102 | kDa | 4 | 8 | 1 | 3 |
| OS = Homo sapiens GN = EIF4G2 PE = 1 SV = 1 | | | | | | |
| Eukaryotic translation initiation factor 6 OS = Homo | 27 | kDa | 4 | 10 | 2 | 5 |
| sapiens OX = 9606 GN = EIF6 PE = 1 SV = 1 | | | | | | |
| Guanine nucleotide-binding protein G(I)/G(S)/G(T) | 37 | kDa | 4 | 12 | 3 | 4 |
| subunit beta-1 OS = Homo sapiens OX = 9606 GN = GNB1 | | | | | | |
| PE = 1 SV = 3 | | | | | | |
| Isoform 10 of Calpastatin OS = Homo sapiens OX = 9606 | 82 | kDa | 4 | 4 | 0 | 1 |
| GN = CAST | | | | | | |
| Isoform 2 of 40S ribosomal protein S24 OS = Homo | 15 | kDa | 4 | 46 | 10 | 8 |
| sapiens OX = 9606 GN = RPS24 | | | | | | |
| Isoform 2 of Chromodomain-helicase-DNA-binding | 221 | kDa | 4 | 5 | 0 | 1 |
| protein 4 OS = Homo sapiens GN = CHD4 | | | | | | |
| Isoform 2 of Eukaryotic translation initiation factor 3 | 61 | kDa | 4 | 5 | 0 | 6 |
| subunit L OS = Homo sapiens OX = 9606 GN = EIF3L | | | | | | |
| Isoform 2 of Glucosidase 2 subunit beta OS = Homo | 59 | kDa | 4 | 5 | 0 | 4 |
| sapiens OX = 9606 GN = PRKCSH | | | | | | |
| Isoform 2 of Hexokinase-1 OS = Homo sapiens OX = 9606 | 102 | kDa | 4 | 3 | 0 | 1 |
| GN = HK1 | | | | | | |
| Isoform 2 of Isocitrate dehydrogenase [NADP], | 45 | kDa | 4 | 19 | 1 | 4 |
| mitochondrial OS = Homo sapiens OX = 9606 GN = IDH2 | | | | | | |
| Isoform 2 of Nodal modulator 2 OS = Homo sapiens | 134 | kDa | 4 | 4 | 0 | 3 |
| OX = 9606 GN = NOMO2 | | | | | | |
| Isoform 2 of Plastin-3 OS = Homo sapiens OX = 9606 | 69 | kDa | 4 | 0 | 0 | 1 |
| GN = PLS3 | | | | | | |
| Isoform 2 of Proteasome subunit alpha type-3 OS = Homo | 28 | kDa | 4 | 6 | 0 | 4 |
| sapiens OX = 9606 GN = PSMA3 | | | | | | |
| Isoform 2 of Protein SET OS = Homo sapiens GN = SET | 32 | kDa | 4 | 7 | 0 | 4 |
| Isoform 2 of Splicing factor U2AF 65 kDa subunit | 53 | kDa | 4 | 1 | 0 | 5 |
| OS = Homo sapiens OX = 9606 GN = U2AF2 | | | | | | |
| Isoform 2 of SWI/SNF complex subunit SMARCC2 | 125 | kDa | 4 | 9 | 0 | 3 |
| OS = Homo sapiens OX = 9606 GN = SMARCC2 | | | | | | |
| Isoform 2 of Unconventional myosin-Ib OS = Homo | 125 | kDa | 4 | 16 | 0 | 0 |
| sapiens OX = 9606 GN = MYO1B | | | | | | |
| Isoform 3 of Dynactin subunit 1 OS = Homo sapiens | 137 | kDa | 4 | 8 | 0 | 2 |
| OX = 9606 GN = DCTN1 | | | | | | |
| Isoform 3 of Heterogeneous nuclear ribonucleoprotein | 31 | kDa | 4 | 10 | 2 | 6 |
| A/B OS = Homo sapiens GN = HNRNPAB | | | | | | |
| Isoform 3 of Myoferlin OS = Homo sapiens OX = 9606 | 233 | kDa | 4 | 6 | 0 | 1 |
| GN = MYOF | | | | | | |
| Isoform 3 of Nucleolar and coiled-body phosphoprotein 1 | 74 | kDa | 4 | 7 | 1 | 3 |
| OS = Homo sapiens OX = 9606 GN = NOLC1 | | | | | | |
| Isoform 4 of Inhibitor of nuclear factor kappa-B kinase- | 43 | kDa | 4 | 11 | 2 | 5 |
| interacting protein OS = Homo sapiens OX = 9606 | | | | | | |
| GN = IKBIP | | | | | | |
| Isoform 6 of MMS19 nucleotide excision repair protein | 108 | kDa | 4 | 0 | 0 | 1 |
| homolog OS = Homo sapiens GN = MMS19 | | | | | | |
| Isoform Short of RNA-binding protein FUS OS = Homo | 53 | kDa | 4 | 9 | 0 | 4 |
| sapiens OX = 9606 GN = FUS | | | | | | |
| Microtubule-actin cross-linking factor 1, isoforms 1/2/3/5 | 838 | kDa | 4 | 2 | 0 | 0 |
| OS = Homo sapiens OX = 9606 GN = MACF1 PE = 1 SV = 4 | | | | | | |
| Mitochondrial carrier homolog 1 (Fragment) OS = Homo | 43 | kDa | 4 | 0 | 0 | 5 |
| sapiens OX = 9606 GN = MTCH1 PE = 1 SV = 1 | | | | | | |
| Nuclease-sensitive element-binding protein 1 OS = Homo | 36 | kDa | 4 | 0 | 7 | 9 |
| sapiens OX = 9606 GN = YBX1 PE = 1 SV = 3 | | | | | | |
| Nucleolar GTP-binding protein 1 OS = Homo sapiens | 74 | kDa | 4 | 5 | 0 | 1 |
| OX = 9606 GN = GTPBP4 PE = 1 SV = 3 | | | | | | |
| OCIA domain-containing protein 2 OS = Homo sapiens | 17 | kDa | 4 | 0 | 0 | 0 |
| OX = 9606 GN = OCIAD2 PE = 1 SV = 1 | | | | | | |
| Peroxisomal multifunctional enzyme type 2 OS = Homo | 80 | kDa | 4 | 10 | 0 | 9 |
| sapiens GN = HSD17B4 PE = 1 SV = 3 | | | | | | |
| Probable ATP-dependent RNA helicase DDX6 | 54 | kDa | 4 | 4 | 0 | 2 |
| OS = Homo sapiens OX = 9606 GN = DDX6 PE = 1 SV = 2 | | | | | | |
| Proteasome subunit alpha type-2 OS = Homo sapiens | 26 | kDa | 4 | 0 | 0 | 8 |
| OX = 9606 GN = PSMA2 PE = 1 SV = 2 | | | | | | |
| Proteasome subunit beta type-1 OS = Homo sapiens | 26 | kDa | 4 | 0 | 0 | 6 |
| OX = 9606 GN = PSMB1 PE = 1 SV = 2 | | | | | | |
| Proteasome subunit beta type-3 OS = Homo sapiens | 23 | kDa | 4 | 0 | 0 | 6 |
| OX = 9606 GN = PSMB3 PE = 1 SV = 2 | | | | | | |
| Proteasome subunit beta type-7 OS = Homo sapiens | 30 | kDa | 4 | 0 | 0 | 6 |
| OX = 9606 GN = PSMB7 PE = 1 SV = 1 | | | | | | |
| Protein arginine N-methyltransferase 1 OS = Homo | 42 | kDa | 4 | 3 | 0 | 1 |
| sapiens GN = PRMT1 PE = 1 SV = 2 | | | | | | |
| Ras GTPase-activating protein-binding protein 2 | 54 | kDa | 4 | 14 | 2 | 5 |
| OS = Homo sapiens OX = 9606 GN = G3BP2 PE = 1 SV = 2 | | | | | | |
| Ras suppressor protein 1 OS = Homo sapiens OX = 9606 | 32 | kDa | 4 | 16 | 1 | 8 |
| GN = RSU1 PE = 1 SV = 3 | | | | | | |
| RNA-binding protein Raly OS = Homo sapiens OX = 9606 | 32 | kDa | 4 | 14 | 2 | 7 |
| GN = RALY PE = 1 SV = 1 | | | | | | |
| Sideroflexin-1 OS = Homo sapiens OX = 9606 GN = SFXN1 | 36 | kDa | 4 | 3 | 1 | 4 |
| PE = 1 SV = 4 | | | | | | |
| Sideroflexin-3 OS = Homo sapiens OX = 9606 GN = SFXN3 | 36 | kDa | 4 | 0 | 0 | 4 |
| PE = 1 SV = 3 | | | | | | |
| Signal recognition particle 9 kDa protein OS = Homo | 10 | kDa | 4 | 7 | 1 | 3 |
| sapiens OX = 9606 GN = SRP9 PE = 1 SV = 2 | | | | | | |
| Splicing factor 3A subunit 1 OS = Homo sapiens | 89 | kDa | 4 | 6 | 0 | 5 |
| OX = 9606 GN = SF3A1 PE = 1 SV = 1 | | | | | | |
| SWISS-PROT: Q9TTE1 (Bos taurus) Endopin-1 | 46 | kDa | 4 | 4 | 2 | 1 |
| precursor | | | | | | |
| Thy-1 membrane glycoprotein OS = Homo sapiens | 18 | kDa | 4 | 0 | 6 | 3 |
| OX = 9606 GN = THY1 PE = 1 SV = 2 | | | | | | |
| Transmembrane protein 43 OS = Homo sapiens OX = 9606 | 45 | kDa | 4 | 0 | 5 | 6 |
| GN = TMEM43 PE = 1 SV = 1 | | | | | | |
| TREMBL: Q1A7A4 (Bos taurus) similar to complement | 189 | kDa | 4 | 0 | 0 | 0 |
| component C5 | | | | | | |
| Tricarboxylate transport protein, mitochondrial | 34 | kDa | 4 | 2 | 0 | 3 |
| OS = Homo sapiens OX = 9606 GN = SLC25A1 PE = 1 SV = 2 | | | | | | |
| U2 small nuclear ribonucleoprotein A′ OS = Homo sapiens | 28 | kDa | 4 | 0 | 0 | 4 |
| OX = 9606 GN = SNRPA1 PE = 1 SV = 2 | | | | | | |
| Vasodilator-stimulated phosphoprotein OS = Homo | 40 | kDa | 4 | 10 | 0 | 10 |
| sapiens OX = 9606 GN = VASP PE = 1 SV = 3 | | | | | | |
| (Bos taurus) 47 kDa protein | 47 | kDa | 3 | 0 | 0 | 0 |
| (Bos taurus) similar to Complement C4-A precursor | 193 | kDa | 3 | 7 | 4 | 4 |
| 26S proteasome non-ATPase regulatory subunit 5 | 56 | kDa | 3 | 4 | 0 | 1 |
| OS = Homo sapiens OX = 9606 GN = PSMD5 PE = 1 SV = 3 | | | | | | |
| 26S proteasome non-ATPase regulatory subunit 7 | 37 | kDa | 3 | 10 | 0 | 6 |
| OS = Homo sapiens OX = 9606 GN = PSMD7 PE = 1 SV = 2 | | | | | | |
| 26S proteasome regulatory subunit 7 OS = Homo sapiens | 49 | kDa | 3 | 4 | 0 | 4 |
| OX = 9606 GN = PSMC2 PE = 1 SV = 3 | | | | | | |
| 39S ribosomal protein L28, mitochondrial OS = Homo | 30 | kDa | 3 | 0 | 0 | 1 |
| sapiens OX = 9606 GN = MRPL28 PE = 1 SV = 4 | | | | | | |
| 40S ribosomal protein S15a OS = Homo sapiens OX = 9606 | 15 | kDa | 3 | 0 | 2 | 3 |
| GN = RPS15A PE = 1 SV = 2 | | | | | | |
| 60S ribosomal protein L15 OS = Homo sapiens OX = 9606 | 24 | kDa | 3 | 27 | 3 | 11 |
| GN = RPL15 PE = 1 SV = 2 | | | | | | |
| Acetyl-CoA acetyltransferase, mitochondrial OS = Homo | 45 | kDa | 3 | 11 | 0 | 5 |
| sapiens OX = 9606 GN = ACAT1 PE = 1 SV = 1 | | | | | | |
| ATP synthase F(0) complex subunit B1, mitochondrial | 29 | kDa | 3 | 0 | 0 | 2 |
| OS = Homo sapiens OX = 9606 GN = ATP5PB PE = 1 SV = 2 | | | | | | |
| ATP-binding cassette sub-family D member 3 OS = Homo | 75 | kDa | 3 | 8 | 2 | 2 |
| sapiens OX = 9606 GN = ABCD3 PE = 1 SV = 1 | | | | | | |
| Calponin-3 OS = Homo sapiens OX = 9606 GN = CNN3 | 36 | kDa | 3 | 4 | 1 | 6 |
| PE = 1 SV = 1 | | | | | | |
| Cell surface glycoprotein MUC18 OS = Homo sapiens | 72 | kDa | 3 | 3 | 0 | 6 |
| OX = 9606 GN = MCAM PE = 1 SV = 2 | | | | | | |
| CTP synthase 1 OS = Homo sapiens OX = 9606 | 67 | kDa | 3 | 0 | 0 | 0 |
| GN = CTPS1 PE = 1 SV = 2 | | | | | | |
| Cytoplasmic dynein 1 light intermediate chain 1 | 57 | kDa | 3 | 11 | 0 | 4 |
| OS = Homo sapiens OX = 9606 GN = DYNC1LI1 PE = 1 | | | | | | |
| SV = 3 | | | | | | |
| Cytoplasmic FMR1-interacting protein 1 OS = Homo | 145 | kDa | 3 | 2 | 0 | 0 |
| sapiens OX = 9606 GN = CYFIP1 PE = 1 SV = 1 | | | | | | |
| Desmoglein-2 OS = Homo sapiens OX = 9606 GN = DSG2 | 122 | kDa | 3 | 17 | 5 | 7 |
| PE = 1 SV = 2 | | | | | | |
| Desmoplakin OS = Homo sapiens OX = 9606 GN = DSP | 332 | kDa | 3 | 35 | 0 | 0 |
| PE = 1 SV = 3 | | | | | | |
| Dihydrolipoyllysine-residue acetyltransferase component | 69 | kDa | 3 | 0 | 2 | 10 |
| of pyruvate dehydrogenase complex, mitochondrial | | | | | | |
| OS = Homo sapiens OX = 9606 GN = DLAT PE = 1 SV = 3 | | | | | | |
| DnaJ homolog subfamily A member 2 OS = Homo sapiens | 46 | kDa | 3 | 0 | 1 | 6 |
| OX = 9606 GN = DNAJA2 PE = 1 SV = 1 | | | | | | |
| Dynein light chain Tctex-type 1 OS = Homo sapiens | 12 | kDa | 3 | 13 | 3 | 5 |
| OX = 9606 GN = DYNLT1 PE = 1 SV = 1 | | | | | | |
| E3 ubiquitin/ISG15 ligase TRIM25 OS = Homo sapiens | 71 | kDa | 3 | 0 | 0 | 1 |
| OX = 9606 GN = TRIM25 PE = 1 SV = 2 | | | | | | |
| EGF-like repeat and discoidin I-like domain-containing | 54 | kDa | 3 | 26 | 1 | 7 |
| protein 3 OS = Homo sapiens OX = 9606 GN = EDIL3 PE = 1 | | | | | | |
| SV = 1 | | | | | | |
| Enoyl-CoA hydratase, mitochondrial OS = Homo sapiens | 31 | kDa | 3 | 0 | 0 | 2 |
| OX = 9606 GN = ECHS1 PE = 1 SV = 4 | | | | | | |
| Epidermal growth factor receptor kinase substrate 8-like | 81 | kDa | 3 | 4 | 0 | 5 |
| protein 2 OS = Homo sapiens OX = 9606 GN = EPS8L2 | | | | | | |
| PE = 1 SV = 2 | | | | | | |
| ER lumen protein-retaining receptor 1 OS = Homo sapiens | 25 | kDa | 3 | 0 | 2 | 1 |
| OX = 9606 GN = KDELR1 PE = 1 SV = 1 | | | | | | |
| Eukaryotic translation initiation factor 2 subunit 2 | 38 | kDa | 3 | 0 | 7 | 10 |
| OS = Homo sapiens OX = 9606 GN = EIF2S2 PE = 1 SV = 2 | | | | | | |
| General transcription factor II-I OS = Homo sapiens | 112 | kDa | 3 | 27 | 3 | 3 |
| OX = 9606 GN = GTF2I PE = 1 SV = 2 | | | | | | |
| High mobility group protein HMGI-C OS = Homo sapiens | 12 | kDa | 3 | 48 | 3 | 0 |
| GN = HMGA2 PE = 1 SV = 1 | | | | | | |
| Inosine-5′-monophosphate dehydrogenase 2 OS = Homo | 56 | kDa | 3 | 0 | 0 | 2 |
| sapiens OX = 9606 GN = IMPDH2 PE = 1 SV = 2 | | | | | | |
| Isoform 1 of Apoptosis inhibitor 5 OS = Homo sapiens | 49 | kDa | 3 | 5 | 0 | 3 |
| OX = 9606 GN = API5 | | | | | | |
| Isoform 12 of Titin OS = Homo sapiens OX = 9606 | 3994 | kDa | 3 | 1 | 3 | 3 |
| GN = TTN | | | | | | |
| Isoform 2 of 3-hydroxyacyl-CoA dehydrogenase type-2 | 26 | kDa | 3 | 2 | 0 | 1 |
| OS = Homo sapiens OX = 9606 GN = HSD17B10 | | | | | | |
| Isoform 2 of ATP-dependent RNA helicase DDX54 | 99 | kDa | 3 | 1 | 0 | 3 |
| OS = Homo sapiens OX = 9606 GN = DDX54 | | | | | | |
| Isoform 2 of B-cell receptor-associated protein 31 | 35 | kDa | 3 | 1 | 4 | 7 |
| OS = Homo sapiens OX = 9606 GN = BCAP31 | | | | | | |
| Isoform 2 of Calcium-binding mitochondrial carrier | 51 | kDa | 3 | 1 | 0 | 5 |
| protein SCaMC-1 OS = Homo sapiens OX = 9606 | | | | | | |
| GN = SLC25A24 | | | | | | |
| Isoform 2 of Collagen alpha-1(V) chain OS = Homo | 184 | kDa | 3 | 0 | 13 | 6 |
| sapiens OX = 9606 GN = COL5A1 | | | | | | |
| Isoform 2 of E3 ubiquitin-protein ligase UBR4 | 576 | kDa | 3 | 0 | 0 | 1 |
| OS = Homo sapiens OX = 9606 GN = UBR4 | | | | | | |
| Isoform 2 of Importin-4 OS = Homo sapiens GN = IPO4 | 119 | kDa | 3 | 0 | 0 | 0 |
| Isoform 2 of Insulin-like growth factor 2 mRNA-binding | 62 | kDa | 3 | 19 | 2 | 5 |
| protein 2 OS = Homo sapiens GN = IGF2BP2 | | | | | | |
| Isoform 2 of NADH dehydrogenase [ubiquinone] 1 alpha | 49 | kDa | 3 | 0 | 0 | 2 |
| subcomplex subunit 10, mitochondrial OS = Homo sapiens | | | | | | |
| OX = 9606 GN = NDUFA10 | | | | | | |
| Isoform 2 of NADH-cytochrome b5 reductase 3 | 32 | kDa | 3 | 6 | 0 | 3 |
| OS = Homo sapiens OX = 9606 GN = CYB5R3 | | | | | | |
| Isoform 2 of Nuclear pore complex protein Nup107 | 103 | kDa | 3 | 1 | 0 | 0 |
| OS = Homo sapiens OX = 9606 GN = NUP107 | | | | | | |
| Isoform 2 of Protein Dok-7 OS = Homo sapiens OX = 9606 | 37 | kDa | 3 | 0 | 2 | 5 |
| GN = DOK7 | | | | | | |
| Isoform 2 of Protein FAM98B OS = Homo sapiens | 46 | kDa | 3 | 9 | 0 | 4 |
| OX = 9606 GN = FAM98B | | | | | | |
| Isoform 2 of Sacsin OS = Homo sapiens OX = 9606 | 437 | kDa | 3 | 0 | 0 | 0 |
| GN = SACS | | | | | | |
| Isoform 2 of Serine/arginine-rich splicing factor 3 | 14 | kDa | 3 | 8 | 0 | 4 |
| OS = Homo sapiens OX = 9606 GN = SRSF3 | | | | | | |
| Isoform 2 of Small nuclear ribonucleoprotein Sm D3 | 13 | kDa | 3 | 10 | 2 | 3 |
| OS = Homo sapiens OX = 9606 GN = SNRPD3 | | | | | | |
| Isoform 2 of Stathmin OS = Homo sapiens OX = 9606 | 20 | kDa | 3 | 0 | 0 | 3 |
| GN = STMN1 | | | | | | |
| Isoform 2 of TP53-binding protein 1 OS = Homo sapiens | 214 | kDa | 3 | 4 | 0 | 0 |
| OX = 9606 GN = TP53BP1 | | | | | | |
| Isoform 2 of Very long-chain specific acyl-CoA | 68 | kDa | 3 | 10 | 0 | 5 |
| dehydrogenase, mitochondrial OS = Homo sapiens | | | | | | |
| OX = 9606 GN = ACADVL | | | | | | |
| Isoform 2 of Voltage-dependent anion-selective channel | 31 | kDa | 3 | 15 | 0 | 5 |
| protein 3 OS = Homo sapiens OX = 9606 GN = VDAC3 | | | | | | |
| Isoform 2 of V-type proton ATPase catalytic subunit A | 65 | kDa | 3 | 15 | 1 | 2 |
| OS = Homo sapiens OX = 9606 GN = ATP6V1A | | | | | | |
| Isoform 3 of 4F2 cell-surface antigen heavy chain | 62 | kDa | 3 | 1 | 1 | 1 |
| OS = Homo sapiens GN = SLC3A2 | | | | | | |
| Isoform 3 of Aldehyde dehydrogenase family 16 member | 80 | kDa | 3 | 0 | 0 | 1 |
| A1 OS = Homo sapiens OX = 9606 GN = ALDH16A1 | | | | | | |
| Isoform 3 of Drebrin OS = Homo sapiens GN = DBN1 | 76 | kDa | 3 | 31 | 4 | 3 |
| Isoform 3 of Erbin OS = Homo sapiens OX = 9606 | 153 | kDa | 3 | 0 | 0 | 0 |
| GN = ERBIN | | | | | | |
| Isoform 3 of Nucleoside diphosphate kinase B OS = Homo | 30 | kDa | 3 | 12 | 0 | 7 |
| sapiens GN = NME2 | | | | | | |
| Isoform 3 of Perilipin-3 OS = Homo sapiens OX = 9606 | 47 | kDa | 3 | 1 | 0 | 3 |
| GN = PLIN3 | | | | | | |
| Isoform 3 of SUN domain-containing protein 2 | 80 | kDa | 3 | 15 | 4 | 12 |
| OS = Homo sapiens OX = 9606 GN = SUN2 | | | | | | |
| Isoform 3 of Transportin-3 OS = Homo sapiens OX = 9606 | 103 | kDa | 3 | 0 | 0 | 0 |
| GN = TNPO3 | | | | | | |
| Isoform 4 of 26S proteasome non-ATPase regulatory | 52 | kDa | 3 | 17 | 0 | 2 |
| subunit 6 OS = Homo sapiens OX = 9606 GN = PSMD6 | | | | | | |
| Isoform 9 of Protein transport protein Sec31A OS = Homo | 131 | kDa | 3 | 3 | 0 | 1 |
| sapiens GN = SEC31A | | | | | | |
| Isoform K of Kinesin light chain 1 OS = Homo sapiens | 70 | kDa | 3 | 2 | 0 | 2 |
| OX = 9606 GN = KLC1 | | | | | | |
| MICOS complex subunit MIC19 OS = Homo sapiens | 26 | kDa | 3 | 0 | 3 | 6 |
| OX = 9606 GN = CHCHD3 PE = 1 SV = 1 | | | | | | |
| Nuclear pore complex protein Nup205 OS = Homo | 228 | kDa | 3 | 0 | 0 | 0 |
| sapiens OX = 9606 GN = NUP205 PE = 1 SV = 3 | | | | | | |
| Nuclear pore complex protein Nup93 OS = Homo sapiens | 93 | kDa | 3 | 6 | 0 | 3 |
| OX = 9606 GN = NUP93 PE = 1 SV = 2 | | | | | | |
| PC4 and SFRS1-interacting protein OS = Homo sapiens | 60 | kDa | 3 | 16 | 1 | 5 |
| OX = 9606 GN = PSIP1 PE = 1 SV = 1 | | | | | | |
| Peroxiredoxin-6 OS = Homo sapiens OX = 9606 | 25 | kDa | 3 | 0 | 0 | 0 |
| GN = PRDX6 PE = 1 SV = 3 | | | | | | |
| PRA1 family protein 3 OS = Homo sapiens OX = 9606 | 22 | kDa | 3 | 7 | 1 | 4 |
| GN = ARL6IP5 PE = 1 SV = 1 | | | | | | |
| Proliferating cell nuclear antigen OS = Homo sapiens | 29 | kDa | 3 | 0 | 0 | 0 |
| OX = 9606 GN = PCNA PE = 1 SV = 1 | | | | | | |
| Proliferation-associated protein 2G4 OS = Homo sapiens | 44 | kDa | 3 | 16 | 0 | 6 |
| OX = 9606 GN = PA2G4 PE = 1 SV = 3 | | | | | | |
| Proteasome adapter and scaffold protein ECM29 | 204 | kDa | 3 | 0 | 0 | 2 |
| OS = Homo sapiens OX = 9606 GN = ECPAS PE = 1 SV = 2 | | | | | | |
| Proteasome subunit beta type-4 OS = Homo sapiens | 29 | kDa | 3 | 0 | 0 | 4 |
| OX = 9606 GN = PSMB4 PE = 1 SV = 4 | | | | | | |
| Proteasome subunit beta type-6 OS = Homo sapiens | 25 | kDa | 3 | 0 | 0 | 3 |
| OX = 9606 GN = PSMB6 PE = 1 SV = 4 | | | | | | |
| Protein DEK OS = Homo sapiens OX = 9606 GN = DEK | 43 | kDa | 3 | 2 | 0 | 2 |
| PE = 1 SV = 1 | | | | | | |
| Protein S100-A11 OS = Homo sapiens OX = 9606 | 12 | kDa | 3 | 0 | 0 | 2 |
| GN = S100A11 PE = 1 SV = 2 | | | | | | |
| Protein transport protein Sec61 subunit beta OS = Homo | 10 | kDa | 3 | 1 | 2 | 2 |
| sapiens OX = 9606 GN = SEC61B PE = 1 SV = 2 | | | | | | |
| Puromycin-sensitive aminopeptidase OS = Homo sapiens | 103 | kDa | 3 | 0 | 0 | 2 |
| GN = NPEPPS PE = 1 SV = 2 | | | | | | |
| Ras-related protein Rab-11B OS = Homo sapiens | 24 | kDa | 3 | 9 | 1 | 6 |
| OX = 9606 GN = RAB11B PE = 1 SV = 4 | | | | | | |
| Ras-related protein Rab-14 OS = Homo sapiens OX = 9606 | 24 | kDa | 3 | 0 | 0 | 6 |
| GN = RAB14 PE = 1 SV = 4 | | | | | | |
| Ras-related protein Rap-1b OS = Homo sapiens OX = 9606 | 21 | kDa | 3 | 7 | 2 | 3 |
| GN = RAP1B PE = 1 SV = 1 | | | | | | |
| Ras-related protein R-Ras OS = Homo sapiens OX = 9606 | 23 | kDa | 3 | 0 | 0 | 2 |
| GN = RRAS PE = 1 SV = 1 | | | | | | |
| RNA transcription, translation and transport factor | 28 | kDa | 3 | 0 | 4 | 6 |
| protein OS = Homo sapiens OX = 9606 GN = RTRAF PE = 1 | | | | | | |
| SV = 1 | | | | | | |
| SAP domain-containing ribonucleoprotein OS = Homo | 24 | kDa | 3 | 0 | 0 | 2 |
| sapiens OX = 9606 GN = SARNP PE = 1 SV = 3 | | | | | | |
| SWISS-PROT: P06868 (Bos taurus) Plasminogen | 91 | kDa | 3 | 1 | 5 | 0 |
| precursor | | | | | | |
| SWISS-PROT: P41361 (Bos taurus) Antithrombin-III | 52 | kDa | 3 | 3 | 0 | 0 |
| precursor | | | | | | |
| TAR DNA-binding protein 43 OS = Homo sapiens | 45 | kDa | 3 | 2 | 0 | 3 |
| OX = 9606 GN = TARDBP PE = 1 SV = 1 | | | | | | |
| Thioredoxin reductase 1, cytoplasmic OS = Homo sapiens | 71 | kDa | 3 | 0 | 0 | 1 |
| GN = TXNRD1 PE = 1 SV = 3 | | | | | | |
| Thrombospondin type-1 domain-containing protein 4 | 112 | kDa | 3 | 22 | 10 | 1 |
| OS = Homo sapiens OX = 9606 GN = THSD4 PE = 2 SV = 2 | | | | | | |
| TREMBL: Q2KJF1 (Bos taurus) Alpha-1-B glycoprotein | 54 | kDa | 3 | 11 | 1 | 3 |
| tRNA-splicing ligase RtcB homolog OS = Homo sapiens | 55 | kDa | 3 | 19 | 3 | 9 |
| OX = 9606 GN = RTCB PE = 1 SV = 1 | | | | | | |
| Tyrosine--tRNA ligase, cytoplasmic OS = Homo sapiens | 59 | kDa | 3 | 0 | 0 | 2 |
| OX = 9606 GN = YARS PE = 1 SV = 4 | | | | | | |
| Ubiquitin thioesterase OTUB1 OS = Homo sapiens | 31 | kDa | 3 | 0 | 0 | 1 |
| OX = 9606 GN = OTUB1 PE = 1 SV = 2 | | | | | | |
| Vigilin OS = Homo sapiens OX = 9606 GN = HDLBP PE = 1 | 141 | kDa | 3 | 9 | 0 | 2 |
| SV = 2 | | | | | | |
| V-type proton ATPase 116 kDa subunit a isoform 3 | 93 | kDa | 3 | 16 | 1 | 2 |
| OS = Homo sapiens OX = 9606 GN = TCIRG1 PE = 1 SV = 3 | | | | | | |
| WD repeat-containing protein 61 OS = Homo sapiens | 34 | kDa | 3 | 0 | 1 | 4 |
| OX = 9606 GN = WDR61 PE = 1 SV = 1 | | | | | | |
| 39S ribosomal protein L41, mitochondrial OS = Homo | 15 | kDa | 2 | 0 | 0 | 5 |
| sapiens OX = 9606 GN = MRPL41 PE = 1 SV = 1 | | | | | | |
| 40S ribosomal protein S25 OS = Homo sapiens OX = 9606 | 14 | kDa | 2 | 0 | 8 | 7 |
| GN = RPS25 PE = 1 SV = 1 | | | | | | |
| 60S acidic ribosomal protein P1 OS = Homo sapiens | 12 | kDa | 2 | 13 | 3 | 5 |
| OX = 9606 GN = RPLP1 PE = 1 SV = 1 | | | | | | |
| Actin-like protein 6A OS = Homo sapiens OX = 9606 | 47 | kDa | 2 | 12 | 0 | 2 |
| GN = ACTL6A PE = 1 SV = 1 | | | | | | |
| Actin-related protein 2/3 complex subunit 5 OS = Homo | 16 | kDa | 2 | 15 | 5 | 7 |
| sapiens OX = 9606 GN = ARPC5 PE = 1 SV = 3 | | | | | | |
| Actin-related protein 2/3 complex subunit 5-like protein | 17 | kDa | 2 | 0 | 6 | 5 |
| OS = Homo sapiens OX = 9606 GN = ARPC5L PE = 1 SV = 1 | | | | | | |
| Activated RNA polymerase II transcriptional coactivator | 14 | kDa | 2 | 1 | 0 | 2 |
| p15 OS = Homo sapiens OX = 9606 GN = SUB1 PE = 1 | | | | | | |
| SV = 3 | | | | | | |
| Alcohol dehydrogenase [NADP(+)] OS = Homo sapiens | 37 | kDa | 2 | 0 | 0 | 1 |
| OX = 9606 GN = AKR1A1 PE = 1 SV = 3 | | | | | | |
| Aspartyl/asparaginyl beta-hydroxylase OS = Homo | 86 | kDa | 2 | 4 | 1 | 3 |
| sapiens OX = 9606 GN = ASPH PE = 1 SV = 3 | | | | | | |
| ATP synthase subunit f, mitochondrial OS = Homo | 11 | kDa | 2 | 0 | 2 | 2 |
| sapiens OX = 9606 GN = ATP5MF PE = 1 SV = 1 | | | | | | |
| ATP-binding cassette sub-family B member 6, | 78 | kDa | 2 | 0 | 0 | 0 |
| mitochondrial (Fragment) OS = Homo sapiens OX = 9606 | | | | | | |
| GN = ABCB6 PE = 1 SV = 1 | | | | | | |
| Catenin delta-1 OS = Homo sapiens OX = 9606 | 108 | kDa | 2 | 13 | 0 | 1 |
| GN = CTNND1 PE = 1 SV = 1 | | | | | | |
| Cathepsin B OS = Homo sapiens OX = 9606 GN = CTSB | 38 | kDa | 2 | 0 | 0 | 0 |
| PE = 1 SV = 3 | | | | | | |
| Caveolin-1 OS = Homo sapiens OX = 9606 GN = CAV1 | 20 | kDa | 2 | 11 | 2 | 4 |
| PE = 1 SV = 4 | | | | | | |
| Cell cycle and apoptosis regulator protein 2 OS = Homo | 103 | kDa | 2 | 1 | 0 | 4 |
| sapiens GN = CCAR2 PE = 1 SV = 2 | | | | | | |
| Centromere protein V OS = Homo sapiens OX = 9606 | 30 | kDa | 2 | 3 | 0 | 1 |
| GN = CENPV PE = 1 SV = 1 | | | | | | |
| Citrate synthase OS = Homo sapiens OX = 9606 GN = CS | 50 | kDa | 2 | 0 | 0 | 3 |
| PE = 1 SV = 1 | | | | | | |
| Cytochrome b-c1 complex subunit 1, mitochondrial | 53 | kDa | 2 | 0 | 0 | 1 |
| OS = Homo sapiens OX = 9606 GN = UQCRC1 PE = 1 SV = 3 | | | | | | |
| Cytochrome c oxidase subunit 7A2, mitochondrial | 9 | kDa | 2 | 0 | 0 | 1 |
| OS = Homo sapiens OX = 9606 GN = COX7A2 PE = 1 SV = 1 | | | | | | |
| Dystonin OS = Homo sapiens OX = 9606 GN = DST PE = 1 | 861 | kDa | 2 | 0 | 0 | 0 |
| SV = 4 | | | | | | |
| EH domain-containing protein 2 OS = Homo sapiens | 61 | kDa | 2 | 2 | 0 | 3 |
| OX = 9606 GN = EHD2 PE = 1 SV = 2 | | | | | | |
| Enhancer of mRNA-decapping protein 4 OS = Homo | 152 | kDa | 2 | 0 | 0 | 0 |
| sapiens GN = EDC4 PE = 1 SV = 1 | | | | | | |
| Eukaryotic translation initiation factor 5B OS = Homo | 139 | kDa | 2 | 0 | 0 | 2 |
| sapiens OX = 9606 GN = EIF5B PE = 1 SV = 4 | | | | | | |
| Exosome RNA helicase MTR4 OS = Homo sapiens | 118 | kDa | 2 | 0 | 0 | 1 |
| OX = 9606 GN = MTREX PE = 1 SV = 3 | | | | | | |
| Far upstream element-binding protein 2 OS = Homo | 73 | kDa | 2 | 0 | 0 | 0 |
| sapiens OX = 9606 GN = KHSRP PE = 1 SV = 4 | | | | | | |
| Ferritin heavy chain OS = Homo sapiens OX = 9606 | 21 | kDa | 2 | 0 | 0 | 1 |
| GN = FTH1 PE = 1 SV = 2 | | | | | | |
| FH2 domain-containing protein 1 OS = Homo sapiens | 125 | kDa | 2 | 0 | 0 | 0 |
| OX = 9606 GN = FHDC1 PE = 1 SV = 2 | | | | | | |
| Glutaredoxin-3 OS = Homo sapiens OX = 9606 | 37 | kDa | 2 | 0 | 0 | 0 |
| GN = GLRX3 PE = 1 SV = 2 | | | | | | |
| GTP-binding protein SAR1a OS = Homo sapiens | 22 | kDa | 2 | 0 | 0 | 3 |
| OX = 9606 GN = SARIA PE = 1 SV = 1 | | | | | | |
| Guanine nucleotide-binding protein G(I)/G(S)/G(T) | 37 | kDa | 2 | 14 | 0 | 0 |
| subunit beta-2 OS = Homo sapiens OX = 9606 GN = GNB2 | | | | | | |
| PE = 1 SV = 3 | | | | | | |
| Heterogeneous nuclear ribonucleoprotein U-like protein 2 | 85 | kDa | 2 | 8 | 0 | 2 |
| OS = Homo sapiens OX = 9606 GN = HNRNPUL2 PE = 1 | | | | | | |
| SV = 1 | | | | | | |
| High mobility group protein B3 OS = Homo sapiens | 23 | kDa | 2 | 0 | 0 | 3 |
| OX = 9606 GN = HMGB3 PE = 1 SV = 4 | | | | | | |
| Inversin OS = Homo sapiens OX = 9606 GN = INVS PE = 1 | 118 | kDa | 2 | 0 | 0 | 0 |
| SV = 2 | | | | | | |
| Isoform 10 of CD44 antigen OS = Homo sapiens | 53 | kDa | 2 | 27 | 5 | 2 |
| OX = 9606 GN = CD44 | | | | | | |
| Isoform 2 of 40S ribosomal protein S20 OS = Homo | 16 | kDa | 2 | 7 | 1 | 3 |
| sapiens OX = 9606 GN = RPS20 | | | | | | |
| Isoform 2 of AP-2 complex subunit alpha-2 OS = Homo | 104 | kDa | 2 | 8 | 0 | 3 |
| sapiens OX = 9606 GN = AP2A2 | | | | | | |
| Isoform 2 of ATP-binding cassette sub-family F member | 92 | kDa | 2 | 6 | 2 | 2 |
| 1 OS = Homo sapiens OX = 9606 GN = ABCF1 | | | | | | |
| Isoform 2 of cAMP-dependent protein kinase type II- | 43 | kDa | 2 | 11 | 0 | 3 |
| alpha regulatory subunit OS = Homo sapiens OX = 9606 | | | | | | |
| GN = PRKAR2A | | | | | | |
| Isoform 2 of Chromatin target of PRMT1 protein | 27 | kDa | 2 | 4 | 2 | 2 |
| OS = Homo sapiens OX = 9606 GN = CHTOP | | | | | | |
| Isoform 2 of Collagen alpha-1(XXII) chain OS = Homo | 159 | kDa | 2 | 0 | 0 | 1 |
| sapiens OX = 9606 GN = COL22A1 | | | | | | |
| Isoform 2 of DNA repair protein RAD50 OS = Homo | 155 | kDa | 2 | 0 | 0 | 1 |
| sapiens OX = 9606 GN = RAD50 | | | | | | |
| Isoform 2 of E3 ubiquitin-protein ligase RNF213 | 596 | kDa | 2 | 0 | 0 | 1 |
| OS = Homo sapiens OX = 9606 GN = RNF213 | | | | | | |
| Isoform 2 of Electron transfer flavoprotein subunit alpha, | 30 | kDa | 2 | 1 | 0 | 5 |
| mitochondrial OS = Homo sapiens OX = 9606 GN = ETFA | | | | | | |
| Isoform 2 of Enoyl-CoA delta isomerase 2, mitochondrial | 40 | kDa | 2 | 0 | 0 | 0 |
| OS = Homo sapiens OX = 9606 GN = ECI2 | | | | | | |
| Isoform 2 of Eukaryotic peptide chain release factor | 45 | kDa | 2 | 2 | 0 | 2 |
| subunit 1 OS = Homo sapiens OX = 9606 GN = ETF1 | | | | | | |
| Isoform 2 of Eukaryotic translation initiation factor 3 | 58 | kDa | 2 | 2 | 0 | 0 |
| subunit D OS = Homo sapiens OX = 9606 GN = EIF3D | | | | | | |
| Isoform 2 of Fermitin family homolog 2 OS = Homo | 72 | kDa | 2 | 0 | 0 | 6 |
| sapiens OX = 9606 GN = FERMT2 | | | | | | |
| Isoform 2 of Glutamine--fructose-6-phosphate | 77 | kDa | 2 | 5 | 0 | 1 |
| aminotransferase [isomerizing] 1 OS = Homo sapiens | | | | | | |
| OX = 9606 GN = GFPT1 | | | | | | |
| Isoform 2 of Guanine nucleotide-binding protein-like 3 | 61 | kDa | 2 | 3 | 0 | 0 |
| OS = Homo sapiens OX = 9606 GN = GNL3 | | | | | | |
| Isoform 2 of Histidine--tRNA ligase, cytoplasmic | 53 | kDa | 2 | 3 | 0 | 4 |
| OS = Homo sapiens OX = 9606 GN = HARS | | | | | | |
| Isoform 2 of Histone H1.0 OS = Homo sapiens OX = 9606 | 19 | kDa | 2 | 1 | 3 | 3 |
| GN = H1F0 | | | | | | |
| Isoform 2 of Interferon-induced, double-stranded RNA- | 57 | kDa | 2 | 1 | 0 | 1 |
| activated protein kinase OS = Homo sapiens OX = 9606 | | | | | | |
| GN = EIF2AK2 | | | | | | |
| Isoform 2 of Neurotrimin OS = Homo sapiens OX = 9606 | 38 | kDa | 2 | 0 | 0 | 0 |
| GN = NTM | | | | | | |
| Isoform 2 of Neutral cholesterol ester hydrolase 1 | 47 | kDa | 2 | 1 | 0 | 3 |
| OS = Homo sapiens OX = 9606 GN = NCEH1 | | | | | | |
| Isoform 2 of Nucleosome assembly protein 1-like 1 | 43 | kDa | 2 | 10 | 1 | 3 |
| OS = Homo sapiens OX = 9606 GN = NAP1L1 | | | | | | |
| Isoform 2 of Nucleosome assembly protein 1-like 4 | 44 | kDa | 2 | 0 | 0 | 3 |
| OS = Homo sapiens OX = 9606 GN = NAP1L4 | | | | | | |
| Isoform 2 of Polyadenylate-binding protein 2 OS = Homo | 31 | kDa | 2 | 7 | 0 | 2 |
| sapiens OX = 9606 GN = PABPN1 | | | | | | |
| Isoform 2 of Pre-mRNA-splicing factor SYF2 OS = Homo | 24 | kDa | 2 | 0 | 0 | 3 |
| sapiens OX = 9606 GN = SYF2 | | | | | | |
| Isoform 2 of Procollagen-lysine, 2-oxoglutarate 5- | 88 | kDa | 2 | 12 | 0 | 1 |
| dioxygenase 1 OS = Homo sapiens OX = 9606 GN = PLOD1 | | | | | | |
| Isoform 2 of Procollagen-lysine, 2-oxoglutarate 5- | 87 | kDa | 2 | 2 | 0 | 0 |
| dioxygenase 2 OS = Homo sapiens OX = 9606 GN = PLOD2 | | | | | | |
| Isoform 2 of Protein enabled homolog OS = Homo sapiens | 64 | kDa | 2 | 26 | 1 | 18 |
| OX = 9606 GN = ENAH | | | | | | |
| Isoform 2 of Protein SGT1 homolog OS = Homo sapiens | 38 | kDa | 2 | 0 | 0 | 0 |
| OX = 9606 GN = SUGT1 | | | | | | |
| Isoform 2 of Protein transport protein Sec16A OS = Homo | 229 | kDa | 2 | 0 | 0 | 1 |
| sapiens OX = 9606 GN = SEC16A | | | | | | |
| Isoform 2 of RNA-binding protein with serine-rich | 32 | kDa | 2 | 3 | 1 | 2 |
| domain 1 OS = Homo sapiens OX = 9606 GN = RNPS1 | | | | | | |
| Isoform 2 of Signal recognition particle subunit SRP68 | 67 | kDa | 2 | 9 | 0 | 0 |
| OS = Homo sapiens OX = 9606 GN = SRP68 | | | | | | |
| Isoform 2 of Syntenin-1 OS = Homo sapiens OX = 9606 | 32 | kDa | 2 | 6 | 0 | 0 |
| GN = SDCBP | | | | | | |
| Isoform 2 of Translocating chain-associated membrane | 40 | kDa | 2 | 6 | 4 | 5 |
| protein 1 OS = Homo sapiens OX = 9606 GN = TRAM1 | | | | | | |
| Isoform 2 of Trifunctional enzyme subunit beta, | 49 | kDa | 2 | 51 | 9 | 10 |
| mitochondrial OS = Homo sapiens OX = 9606 | | | | | | |
| GN = HADHB | | | | | | |
| Isoform 2 of UDP-glucose: glycoprotein | 175 | kDa | 2 | 2 | 0 | 1 |
| glucosyltransferase 1 OS = Homo sapiens OX = 9606 | | | | | | |
| GN = UGGT1 | | | | | | |
| Isoform 3 of 28S ribosomal protein S29, mitochondrial | 42 | kDa | 2 | 5 | 2 | 1 |
| OS = Homo sapiens GN = DAP3 | | | | | | |
| Isoform 3 of Basic leucine zipper and W2 domain- | 51 | kDa | 2 | 1 | 0 | 4 |
| containing protein 1 OS = Homo sapiens OX = 9606 | | | | | | |
| GN = BZW1 | | | | | | |
| Isoform 3 of E3 ubiquitin-protein ligase HUWE1 | 481 | kDa | 2 | 4 | 0 | 0 |
| OS = Homo sapiens GN = HUWE1 | | | | | | |
| Isoform 3 of Heterogeneous nuclear ribonucleoprotein | 32 | kDa | 2 | 8 | 0 | 3 |
| H3 OS = Homo sapiens GN = HNRNPH3 | | | | | | |
| Isoform 3 of Integrin-linked protein kinase OS = Homo | 36 | kDa | 2 | 0 | 0 | 2 |
| sapiens OX = 9606 GN = ILK | | | | | | |
| Isoform 3 of Protein virilizer homolog OS = Homo sapiens | 201 | kDa | 2 | 0 | 0 | 0 |
| OX = 9606 GN = VIRMA | | | | | | |
| Isoform 3 of Ubiquitin-associated protein 2-like | 113 | kDa | 2 | 7 | 0 | 7 |
| OS = Homo sapiens OX = 9606 GN = UBAP2L | | | | | | |
| Isoform 4 of LIM domain and actin-binding protein 1 | 85 | kDa | 2 | 49 | 6 | 4 |
| OS = Homo sapiens OX = 9606 GN = LIMA1 | | | | | | |
| Isoform 4 of WASH complex subunit 2C OS = Homo | 145 | kDa | 2 | 0 | 0 | 0 |
| sapiens OX = 9606 GN = WASHC2C | | | | | | |
| Isoform Delta 10 of Calcium/calmodulin-dependent | 56 | kDa | 2 | 5 | 0 | 0 |
| protein kinase type II subunit delta OS = Homo sapiens | | | | | | |
| OX = 9606 GN = CAMK2D | | | | | | |
| Isoform LAMP-2B of Lysosome-associated membrane | 45 | kDa | 2 | 0 | 0 | 2 |
| glycoprotein 2 OS = Homo sapiens OX = 9606 | | | | | | |
| GN = LAMP2 | | | | | | |
| Leucine-rich repeat flightless-interacting protein 1 | 89 | kDa | 2 | 0 | 0 | 1 |
| OS = Homo sapiens OX = 9606 GN = LRRFIP1 PE = 1 SV = 2 | | | | | | |
| Lysophospholipid acyltransferase 7 OS = Homo sapiens | 53 | kDa | 2 | 2 | 0 | 0 |
| GN = MBOAT7 PE = 1 SV = 2 | | | | | | |
| Microsomal glutathione S-transferase 3 OS = Homo | 17 | kDa | 2 | 1 | 1 | 2 |
| sapiens OX = 9606 GN = MGST3 PE = 1 SV = 1 | | | | | | |
| Mitochondrial import receptor subunit TOM40 homolog | 38 | kDa | 2 | 0 | 0 | 0 |
| OS = Homo sapiens OX = 9606 GN = TOMM40 PE = 1 | | | | | | |
| SV = 1 | | | | | | |
| Mucin-19 OS = Homo sapiens OX = 9606 GN = MUC19 | 805 | kDa | 2 | 0 | 0 | 0 |
| PE = 1 SV = 3 | | | | | | |
| NADH dehydrogenase [ubiquinone] iron-sulfur protein 3, | 30 | kDa | 2 | 4 | 0 | 4 |
| mitochondrial OS = Homo sapiens OX = 9606 | | | | | | |
| GN = NDUFS3 PE = 1 SV = 1 | | | | | | |
| Nascent polypeptide-associated complex subunit alpha, | 205 | kDa | 2 | 6 | 0 | 4 |
| muscle-specific form OS = Homo sapiens OX = 9606 | | | | | | |
| GN = NACA PE = 1 SV = 1 | | | | | | |
| Nicotinamide phosphoribosyltransferase OS = Homo | 56 | kDa | 2 | 2 | 0 | 3 |
| sapiens OX = 9606 GN = NAMPT PE = 1 SV = 1 | | | | | | |
| Non-histone chromosomal protein HMG-14 OS = Homo | 12 | kDa | 2 | 11 | 3 | 6 |
| sapiens OX = 9606 GN = HMGN1 PE = 1 SV = 1 | | | | | | |
| Nucleolar protein 11 OS = Homo sapiens OX = 9606 | 81 | kDa | 2 | 0 | 0 | 0 |
| GN = NOL11 PE = 1 SV = 1 | | | | | | |
| Obg-like ATPase 1 OS = Homo sapiens OX = 9606 | 45 | kDa | 2 | 4 | 0 | 3 |
| GN = OLA1 PE = 1 SV = 2 | | | | | | |
| PDZ and LIM domain protein 5 OS = Homo sapiens | 64 | kDa | 2 | 3 | 0 | 2 |
| GN = PDLIM5 PE = 1 SV = 5 | | | | | | |
| Peroxiredoxin-2 OS = Homo sapiens OX = 9606 | 22 | kDa | 2 | 7 | 0 | 1 |
| GN = PRDX2 PE = 1 SV = 5 | | | | | | |
| Phospholipid-transporting ATPase IB OS = Homo sapiens | 129 | kDa | 2 | 0 | 2 | 0 |
| OX = 9606 GN = ATP8A2 PE = 1 SV = 3 | | | | | | |
| Phosphoserine aminotransferase OS = Homo sapiens | 40 | kDa | 2 | 0 | 0 | 0 |
| OX = 9606 GN = PSAT1 PE = 1 SV = 2 | | | | | | |
| Plasminogen activator inhibitor 1 OS = Homo sapiens | 45 | kDa | 2 | 90 | 41 | 29 |
| OX = 9606 GN = SERPINE1 PE = 1 SV = 1 | | | | | | |
| pre-rRNA processing protein FTSJ3 OS = Homo sapiens | 97 | kDa | 2 | 3 | 0 | 1 |
| OX = 9606 GN = FTSJ3 PE = 1 SV = 2 | | | | | | |
| PRKC apoptosis WT1 regulator protein OS = Homo | 37 | kDa | 2 | 0 | 1 | 6 |
| sapiens OX = 9606 GN = PAWR PE = 1 SV = 1 | | | | | | |
| Proteasome activator complex subunit 2 OS = Homo | 27 | kDa | 2 | 0 | 0 | 2 |
| sapiens OX = 9606 GN = PSME2 PE = 1 SV = 4 | | | | | | |
| Proteasome subunit alpha type-5 OS = Homo sapiens | 26 | kDa | 2 | 2 | 0 | 2 |
| OX = 9606 GN = PSMA5 PE = 1 SV = 3 | | | | | | |
| Proteasome subunit alpha type-7 OS = Homo sapiens | 28 | kDa | 2 | 7 | 0 | 2 |
| OX = 9606 GN = PSMA7 PE = 1 SV = 1 | | | | | | |
| Proteasome subunit beta type-2 OS = Homo sapiens | 23 | kDa | 2 | 0 | 0 | 4 |
| OX = 9606 GN = PSMB2 PE = 1 SV = 1 | | | | | | |
| Protein flightless-1 homolog OS = Homo sapiens | 145 | kDa | 2 | 0 | 0 | 1 |
| OX = 9606 GN = FLII PE = 1 SV = 2 | | | | | | |
| Protein SEC13 homolog OS = Homo sapiens OX = 9606 | 36 | kDa | 2 | 4 | 0 | 4 |
| GN = SEC13 PE = 1 SV = 3 | | | | | | |
| Ras-related protein Rab-5C OS = Homo sapiens OX = 9606 | 23 | kDa | 2 | 7 | 0 | 3 |
| GN = RAB5C PE = 1 SV = 2 | | | | | | |
| Ras-related protein Rab-7a OS = Homo sapiens OX = 9606 | 23 | kDa | 2 | 0 | 0 | 2 |
| GN = RAB7A PE = 1 SV = 1 | | | | | | |
| Remodeling and spacing factor 1 OS = Homo sapiens | 164 | kDa | 2 | 9 | 0 | 0 |
| GN = RSF1 PE = 1 SV = 2 | | | | | | |
| Serine beta-lactamase-like protein LACTB, | 61 | kDa | 2 | 13 | 5 | 3 |
| mitochondrial OS = Homo sapiens OX = 9606 GN = LACTB | | | | | | |
| PE = 1 SV = 2 | | | | | | |
| Serine/arginine-rich splicing factor 1 OS = Homo sapiens | 28 | kDa | 2 | 7 | 0 | 2 |
| GN = SRSF1 PE = 1 SV = 2 | | | | | | |
| Structural maintenance of chromosomes protein 1A | 143 | kDa | 2 | 7 | 0 | 0 |
| OS = Homo sapiens OX = 9606 GN = SMC1A PE = 1 SV = 2 | | | | | | |
| SWISS-PROT: P02777 (Bos taurus) similar to Platelet | 24 | kDa | 2 | 3 | 3 | 3 |
| factor 4 | | | | | | |
| SWISS-PROT: Q2UVX4 (Bos taurus) Complement C3 | 187 | kDa | 2 | 7 | 6 | 4 |
| precursor | | | | | | |
| SWISS-PROT: Q95121 (Bos taurus) Pigment epithelium- | 46 | kDa | 2 | 0 | 3 | 3 |
| derived factor precursor | | | | | | |
| Transaldolase OS = Homo sapiens OX = 9606 | 38 | kDa | 2 | 0 | 0 | 3 |
| GN = TALDO1 PE = 1 SV = 2 | | | | | | |
| Translationally-controlled tumor protein OS = Homo | 20 | kDa | 2 | 0 | 0 | 2 |
| sapiens OX = 9606 GN = TPT1 PE = 1 SV = 1 | | | | | | |
| Translocon-associated protein subunit alpha OS = Homo | 32 | kDa | 2 | 5 | 3 | 5 |
| sapiens OX = 9606 GN = SSR1 PE = 1 SV = 3 | | | | | | |
| Transmembrane emp24 domain-containing protein 10 | 25 | kDa | 2 | 0 | 6 | 8 |
| OS = Homo sapiens OX = 9606 GN = TMED10 PE = 1 SV = 2 | | | | | | |
| Transmembrane emp24 domain-containing protein 9 | 27 | kDa | 2 | 0 | 1 | 2 |
| OS = Homo sapiens OX = 9606 GN = TMED9 PE = 1 SV = 2 | | | | | | |
| TREMBL: Q1RMN8 (Bos taurus) Similar to | 25 | kDa | 2 | 0 | 0 | 0 |
| Immunoglobulin lambda-like polypeptide 1 | | | | | | |
| Tripeptidyl-peptidase 1 OS = Homo sapiens GN = TPP1 | 61 | kDa | 2 | 0 | 2 | 3 |
| PE = 1 SV = 2 | | | | | | |
| 1,4-alpha-glucan-branching enzyme OS = Homo sapiens | 80 | kDa | 1 | 0 | 0 | 3 |
| OX = 9606 GN = GBE1 PE = 1 SV = 3 | | | | | | |
| 1-phosphatidylinositol 4,5-bisphosphate | 139 | kDa | 1 | 0 | 0 | 2 |
| phosphodiesterase beta-3 OS = Homo sapiens GN = PLCB3 | | | | | | |
| PE = 1 SV = 2 | | | | | | |
| 28S ribosomal protein S35, mitochondrial OS = Homo | 37 | kDa | 1 | 2 | 0 | 2 |
| sapiens GN = MRPS35 PE = 1 SV = 1 | | | | | | |
| 40S ribosomal protein S30 OS = Homo sapiens OX = 9606 | 7 | kDa | 1 | 0 | 3 | 2 |
| GN = FAU PE = 1 SV = 1 | | | | | | |
| 60S ribosomal protein L11 OS = Homo sapiens OX = 9606 | 20 | kDa | 1 | 18 | 1 | 3 |
| GN = RPL11 PE = 1 SV = 2 | | | | | | |
| 60S ribosomal protein L35a OS = Homo sapiens | 13 | kDa | 1 | 0 | 2 | 2 |
| OX = 9606 GN = RPL35A PE = 1 SV = 2 | | | | | | |
| 60S ribosomal protein L37a OS = Homo sapiens | 10 | kDa | 1 | 0 | 5 | 5 |
| OX = 9606 GN = RPL37A PE = 1 SV = 2 | | | | | | |
| Alpha-parvin OS = Homo sapiens OX = 9606 GN = PARVA | 42 | kDa | 1 | 3 | 0 | 4 |
| PE = 1 SV = 1 | | | | | | |
| AT-rich interactive domain-containing protein 1A | 242 | kDa | 1 | 3 | 0 | 0 |
| OS = Homo sapiens OX = 9606 GN = ARID1A PE = 1 SV = 3 | | | | | | |
| Basigin OS = Homo sapiens OX = 9606 GN = BSG PE = 1 | 42 | kDa | 1 | 3 | 0 | 1 |
| SV = 2 | | | | | | |
| Biorientation of chromosomes in cell division protein 1- | 330 | kDa | 1 | 0 | 0 | 3 |
| like 1 OS = Homo sapiens OX = 9606 GN = BOD1L1 PE = 1 | | | | | | |
| SV = 2 | | | | | | |
| Cell division cycle 5-like protein OS = Homo sapiens | 92 | kDa | 1 | 3 | 0 | 4 |
| OX = 9606 GN = CDC5L PE = 1 SV = 2 | | | | | | |
| Coactosin-like protein OS = Homo sapiens OX = 9606 | 16 | kDa | 1 | 0 | 0 | 2 |
| GN = COTL1 PE = 1 SV = 3 | | | | | | |
| Collagen alpha-1(III) chain OS = Homo sapiens OX = 9606 | 139 | kDa | 1 | 2 | 0 | 1 |
| GN = COL3A1 PE = 1 SV = 4 | | | | | | |
| Collagen alpha-2(I) chain OS = Homo sapiens OX = 9606 | 129 | kDa | 1 | 12 | 7 | 3 |
| GN = COL1A2 PE = 1 SV = 7 | | | | | | |
| Collagen alpha-2(V) chain OS = Homo sapiens OX = 9606 | 145 | kDa | 1 | 0 | 15 | 6 |
| GN = COL5A2 PE = 1 SV = 3 | | | | | | |
| Collagen alpha-6(VI) chain OS = Homo sapiens OX = 9606 | 247 | kDa | 1 | 0 | 3 | 2 |
| GN = COL6A6 PE = 1 SV = 2 | | | | | | |
| Complement component 1 Q subcomponent-binding | 31 | kDa | 1 | 0 | 0 | 4 |
| protein, mitochondrial OS = Homo sapiens OX = 9606 | | | | | | |
| GN = C1QBP PE = 1 SV = 1 | | | | | | |
| Cysteine and glycine-rich protein 1 OS = Homo sapiens | 21 | kDa | 1 | 0 | 0 | 2 |
| OX = 9606 GN = CSRP1 PE = 1 SV = 3 | | | | | | |
| Cytochrome b-c1 complex subunit 9 OS = Homo sapiens | 7 | kDa | 1 | 2 | 0 | 2 |
| OX = 9606 GN = UQCR10 PE = 1 SV = 3 | | | | | | |
| Cytochrome c1, heme protein, mitochondrial OS = Homo | 35 | kDa | 1 | 0 | 0 | 3 |
| sapiens OX = 9606 GN = CYC1 PE = 1 SV = 3 | | | | | | |
| DDRGK domain-containing protein 1 OS = Homo sapiens | 36 | kDa | 1 | 0 | 1 | 2 |
| OX = 9606 GN = DDRGK1 PE = 1 SV = 2 | | | | | | |
| Destrin OS = Homo sapiens OX = 9606 GN = DSTN PE = 1 | 19 | kDa | 1 | 3 | 0 | 6 |
| SV = 3 | | | | | | |
| Dihydrolipoyl dehydrogenase, mitochondrial OS = Homo | 54 | kDa | 1 | 8 | 0 | 2 |
| sapiens OX = 9606 GN = DLD PE = 1 SV = 2 | | | | | | |
| DNA-directed RNA polymerases I, II, and III subunit | 25 | kDa | 1 | 0 | 5 | 1 |
| RPABC1 OS = Homo sapiens OX = 9606 GN = POLR2E | | | | | | |
| PE = 1 SV = 4 | | | | | | |
| ELAV-like protein 1 OS = Homo sapiens OX = 9606 | 36 | kDa | 1 | 3 | 0 | 1 |
| GN = ELAVL1 PE = 1 SV = 2 | | | | | | |
| Eukaryotic translation initiation factor 3 subunit G | 36 | kDa | 1 | 0 | 0 | 3 |
| OS = Homo sapiens OX = 9606 GN = EIF3G PE = 1 SV = 2 | | | | | | |
| Farnesyl pyrophosphate synthase OS = Homo sapiens | 48 | kDa | 1 | 3 | 0 | 1 |
| OX = 9606 GN = FDPS PE = 1 SV = 4 | | | | | | |
| Fibrillin-1 OS = Homo sapiens OX = 9606 GN = FBN1 | 312 | kDa | 1 | 0 | 2 | 0 |
| PE = 1 SV = 3 | | | | | | |
| Fumarate hydratase, mitochondrial OS = Homo sapiens | 55 | kDa | 1 | 0 | 0 | 2 |
| GN = FH PE = 1 SV = 3 | | | | | | |
| Growth/differentiation factor 15 OS = Homo sapiens | 34 | kDa | 1 | 0 | 7 | 6 |
| OX = 9606 GN = GDF15 PE = 1 SV = 3 | | | | | | |
| Histone deacetylase complex subunit SAP18 OS = Homo | 20 | kDa | 1 | 0 | 0 | 2 |
| sapiens OX = 9606 GN = SAP18 PE = 1 SV = 1 | | | | | | |
| Histone H1x OS = Homo sapiens OX = 9606 GN = H1FX | 22 | kDa | 1 | 0 | 0 | 2 |
| PE = 1 SV = 1 | | | | | | |
| Hydrocephalus-inducing protein homolog OS = Homo | 576 | kDa | 1 | 0 | 1 | 0 |
| sapiens OX = 9606 GN = HYDIN PE = 1 SV = 3 | | | | | | |
| Insulin-like growth factor 2 mRNA-binding protein 3 | 64 | kDa | 1 | 18 | 2 | 0 |
| OS = Homo sapiens GN = IGF2BP3 PE = 1 SV = 2 | | | | | | |
| Isoform 2 of Adipocyte plasma membrane-associated | 32 | kDa | 1 | 0 | 0 | 3 |
| protein OS = Homo sapiens OX = 9606 GN = APMAP | | | | | | |
| Isoform 2 of Aspartate aminotransferase, mitochondrial | 43 | kDa | 1 | 0 | 0 | 3 |
| OS = Homo sapiens OX = 9606 GN = GOT2 | | | | | | |
| Isoform 2 of BH3-interacting domain death agonist | 27 | kDa | 1 | 4 | 0 | 0 |
| OS = Homo sapiens OX = 9606 GN = BID | | | | | | |
| Isoform 2 of DnaJ homolog subfamily A member 3, | 50 | kDa | 1 | 0 | 0 | 3 |
| mitochondrial OS = Homo sapiens OX = 9606 | | | | | | |
| GN = DNAJA3 | | | | | | |
| Isoform 2 of Glia-derived nexin OS = Homo sapiens | 44 | kDa | 1 | 8 | 0 | 0 |
| OX = 9606 GN = SERPINE2 | | | | | | |
| Isoform 2 of Histone deacetylase 2 OS = Homo sapiens | 52 | kDa | 1 | 4 | 0 | 1 |
| OX = 9606 GN = HDAC2 | | | | | | |
| Isoform 2 of Lysosomal protective protein OS = Homo | 52 | kDa | 1 | 1 | 0 | 2 |
| sapiens OX = 9606 GN = CTSA | | | | | | |
| Isoform 2 of Lysosome membrane protein 2 OS = Homo | 38 | kDa | 1 | 4 | 0 | 0 |
| sapiens OX = 9606 GN = SCARB2 | | | | | | |
| Isoform 2 of Myosin phosphatase Rho-interacting protein | 118 | kDa | 1 | 13 | 0 | 0 |
| OS = Homo sapiens OX = 9606 GN = MPRIP | | | | | | |
| Isoform 2 of NADH dehydrogenase [ubiquinone] 1 alpha | 25 | kDa | 1 | 13 | 1 | 3 |
| subcomplex subunit 13 OS = Homo sapiens OX = 9606 | | | | | | |
| GN = NDUFA13 | | | | | | |
| Isoform 2 of NADH dehydrogenase [ubiquinone] iron- | 52 | kDa | 1 | 5 | 0 | 1 |
| sulfur protein 2, mitochondrial OS = Homo sapiens | | | | | | |
| OX = 9606 GN = NDUFS2 | | | | | | |
| Isoform 2 of Nesprin-2 OS = Homo sapiens OX = 9606 | 799 | kDa | 1 | 12 | 1 | 0 |
| GN = SYNE2 | | | | | | |
| Isoform 2 of Nuclear pore complex protein Nup214 | 213 | kDa | 1 | 0 | 0 | 3 |
| OS = Homo sapiens OX = 9606 GN = NUP214 | | | | | | |
| Isoform 2 of Phenylalanine--tRNA ligase alpha subunit | 54 | kDa | 1 | 7 | 1 | 4 |
| OS = Homo sapiens OX = 9606 GN = FARSA | | | | | | |
| Isoform 2 of Probable 28S rRNA (cytosine(4447)-C(5))- | 89 | kDa | 1 | 6 | 0 | 6 |
| methyltransferase OS = Homo sapiens OX = 9606 | | | | | | |
| GN = NOP2 | | | | | | |
| Isoform 2 of Protein FAM98A OS = Homo sapiens | 55 | kDa | 1 | 2 | 0 | 3 |
| OX = 9606 GN = FAM98A | | | | | | |
| Isoform 2 of Protein TFG OS = Homo sapiens OX = 9606 | 43 | kDa | 1 | 4 | 0 | 4 |
| GN = TFG | | | | | | |
| Isoform 2 of Pyruvate dehydrogenase E1 component | 37 | kDa | 1 | 7 | 0 | 3 |
| subunit beta, mitochondrial OS = Homo sapiens OX = 9606 | | | | | | |
| GN = PDHB | | | | | | |
| Isoform 2 of UTP--glucose-1-phosphate | 56 | kDa | 1 | 3 | 0 | 7 |
| uridylyltransferase OS = Homo sapiens OX = 9606 | | | | | | |
| GN = UGP2 | | | | | | |
| Isoform 2 of V-type proton ATPase 116 kDa subunit a | 96 | kDa | 1 | 11 | 0 | 3 |
| isoform 1 OS = Homo sapiens OX = 9606 GN = ATP6V0A1 | | | | | | |
| Isoform 2B of Cytoplasmic dynein 1 intermediate chain 2 | 71 | kDa | 1 | 16 | 1 | 3 |
| OS = Homo sapiens OX = 9606 GN = DYNC1I2 | | | | | | |
| Isoform 3 of Activating signal cointegrator 1 complex | 77 | kDa | 1 | 6 | 0 | 1 |
| subunit 2 OS = Homo sapiens OX = 9606 GN = ASCC2 | | | | | | |
| Isoform 3 of Apoptosis-inducing factor 1, mitochondrial | 66 | kDa | 1 | 3 | 0 | 4 |
| OS = Homo sapiens OX = 9606 GN = AIFM1 | | | | | | |
| Isoform 3 of Calumenin OS = Homo sapiens OX = 9606 | 38 | kDa | 1 | 1 | 0 | 2 |
| GN = CALU | | | | | | |
| Isoform 3 of Fragile X mental retardation syndrome- | 60 | kDa | 1 | 4 | 0 | 1 |
| related protein 1 OS = Homo sapiens OX = 9606 | | | | | | |
| GN = FXR1 | | | | | | |
| Isoform 3 of GRB 10-interacting GYF protein 2 | 149 | kDa | 1 | 0 | 0 | 0 |
| OS = Homo sapiens OX = 9606 GN = GIGYF2 | | | | | | |
| Isoform 3 of Pyrroline-5-carboxylate reductase 1, | 36 | kDa | 1 | 8 | 0 | 0 |
| mitochondrial OS = Homo sapiens OX = 9606 GN = PYCR1 | | | | | | |
| Isoform 3 of Ras-related protein R-Ras2 OS = Homo | 20 | kDa | 1 | 5 | 0 | 1 |
| sapiens GN = RRAS2 | | | | | | |
| Isoform 3 of Signal peptidase complex catalytic subunit | 21 | kDa | 1 | 2 | 1 | 3 |
| SEC11A OS = Homo sapiens OX = 9606 GN = SEC11A | | | | | | |
| Isoform 4 of Cadherin-13 OS = Homo sapiens OX = 9606 | 83 | kDa | 1 | 23 | 6 | 2 |
| GN = CDH13 | | | | | | |
| Isoform 4 of Dipeptidyl peptidase 3 OS = Homo sapiens | 79 | kDa | 1 | 0 | 0 | 2 |
| OX = 9606 GN = DPP3 | | | | | | |
| Isoform 4 of Dynamin-1-like protein OS = Homo sapiens | 81 | kDa | 1 | 2 | 0 | 2 |
| OX = 9606 GN = DNM1L | | | | | | |
| Isoform 4 of Dynamin-2 OS = Homo sapiens OX = 9606 | 98 | kDa | 1 | 13 | 0 | 6 |
| GN = DNM2 | | | | | | |
| Isoform 4 of Nexilin OS = Homo sapiens OX = 9606 | 73 | kDa | 1 | 28 | 12 | 7 |
| GN = NEXN | | | | | | |
| Isoform 5 of Obscurin OS = Homo sapiens OX = 9606 | 925 | kDa | 1 | 0 | 0 | 1 |
| GN = OBSCN | | | | | | |
| Isoform 6 of RNA-binding protein EWS OS = Homo | 63 | kDa | 1 | 6 | 0 | 6 |
| sapiens OX = 9606 GN = EWSR1 | | | | | | |
| Isoform Heart of ATP synthase subunit gamma, | 33 | kDa | 1 | 6 | 0 | 4 |
| mitochondrial OS = Homo sapiens OX = 9606 | | | | | | |
| GN = ATP5F1C | | | | | | |
| Isoform Mitochondrial of Lysine--tRNA ligase | 71 | kDa | 1 | 4 | 0 | 1 |
| OS = Homo sapiens OX = 9606 GN = KARS | | | | | | |
| Leucine-rich repeat-containing protein 17 OS = Homo | 52 | kDa | 1 | 11 | 9 | 4 |
| sapiens OX = 9606 GN = LRRC17 PE = 2 SV = 1 | | | | | | |
| Leucyl-cystinyl aminopeptidase OS = Homo sapiens | 117 | kDa | 1 | 4 | 3 | 2 |
| OX = 9606 GN = LNPEP PE = 1 SV = 3 | | | | | | |
| LIM and SH3 domain protein 1 OS = Homo sapiens | 30 | kDa | 1 | 1 | 0 | 3 |
| OX = 9606 GN = LASP1 PE = 1 SV = 2 | | | | | | |
| Matrix metalloproteinase-14 OS = Homo sapiens | 66 | kDa | 1 | 0 | 0 | 2 |
| OX = 9606 GN = MMP14 PE = 1 SV = 3 | | | | | | |
| Microtubule-associated protein RP/EB family member 1 | 30 | kDa | 1 | 4 | 0 | 4 |
| OS = Homo sapiens OX = 9606 GN = MAPRE1 PE = 1 SV = 3 | | | | | | |
| Mitochondrial 2-oxoglutarate/malate carrier protein | 34 | kDa | 1 | 7 | 0 | 1 |
| OS = Homo sapiens OX = 9606 GN = SLC25A11 PE = 1 | | | | | | |
| SV = 3 | | | | | | |
| Mitochondrial fission 1 protein OS = Homo sapiens | 17 | kDa | 1 | 0 | 0 | 3 |
| OX = 9606 GN = FIS1 PE = 1 SV = 2 | | | | | | |
| NADH dehydrogenase [ubiquinone] 1 alpha subcomplex | 17 | kDa | 1 | 6 | 1 | 2 |
| subunit 12 OS = Homo sapiens OX = 9606 GN = NDUFA12 | | | | | | |
| PE = 1 SV = 1 | | | | | | |
| PDZ domain-containing protein 4 OS = Homo sapiens | 86 | kDa | 1 | 0 | 0 | 0 |
| OX = 9606 GN = PDZD4 PE = 1 SV = 1 | | | | | | |
| Peroxisomal membrane protein PEX14 OS = Homo | 41 | kDa | 1 | 3 | 0 | 2 |
| sapiens OX = 9606 GN = PEX14 PE = 1 SV = 1 | | | | | | |
| Platelet-activating factor acetylhydrolase IB subunit beta | 26 | kDa | 1 | 6 | 0 | 1 |
| OS = Homo sapiens GN = PAFAH1B2 PE = 1 SV = 1 | | | | | | |
| Podocalyxin OS = Homo sapiens GN = PODXL PE = 1 | 59 | kDa | 1 | 0 | 0 | 2 |
| SV = 2 | | | | | | |
| Prolyl 3-hydroxylase 3 OS = Homo sapiens OX = 9606 | 82 | kDa | 1 | 0 | 0 | 2 |
| GN = P3H3 PE = 1 SV = 1 | | | | | | |
| Prolyl 4-hydroxylase subunit alpha-2 OS = Homo sapiens | 61 | kDa | 1 | 2 | 0 | 0 |
| OX = 9606 GN = P4HA2 PE = 1 SV = 1 | | | | | | |
| Proteasome subunit alpha type-6 OS = Homo sapiens | 27 | kDa | 1 | 8 | 0 | 1 |
| OX = 9606 GN = PSMA6 PE = 1 SV = 1 | | | | | | |
| Proteasome subunit beta type-5 OS = Homo sapiens | 28 | kDa | 1 | 2 | 0 | 0 |
| GN = PSMB5 PE = 1 SV = 3 | | | | | | |
| Protein CYR61 OS = Homo sapiens OX = 9606 | 42 | kDa | 1 | 0 | 27 | 24 |
| GN = CYR61 PE = 1 SV = 1 | | | | | | |
| Protein mago nashi homolog OS = Homo sapiens | 17 | kDa | 1 | 7 | 0 | 0 |
| OX = 9606 GN = MAGOH PE = 1 SV = 1 | | | | | | |
| RNA-binding protein 14 OS = Homo sapiens OX = 9606 | 69 | kDa | 1 | 18 | 2 | 5 |
| GN = RBM14 PE = 1 SV = 2 | | | | | | |
| RuvB-like 1 OS = Homo sapiens OX = 9606 GN = RUVBL1 | 50 | kDa | 1 | 18 | 0 | 2 |
| PE = 1 SV = 1 | | | | | | |
| Splicing factor 3B subunit 4 OS = Homo sapiens | 44 | kDa | 1 | 0 | 0 | 2 |
| OX = 9606 GN = SF3B4 PE = 1 SV = 1 | | | | | | |
| Splicing factor U2AF 26 kDa subunit OS = Homo sapiens | 4 | kDa | 1 | 0 | 2 | 1 |
| OX = 9606 GN = U2AF1L4 PE = 4 SV = 1 | | | | | | |
| SWISS-PROT: P04258 (Bos taurus) Similar to Collagen | 138 | kDa | 1 | 9 | 0 | 0 |
| alpha 1(III) chain | | | | | | |
| TREMBL: A2I7N3; Q27984 (Bos taurus) SERPINA3-7 | 47 | kDa | 1 | 5 | 1 | 1 |
| Unconventional myosin-Ie OS = Homo sapiens OX = 9606 | 127 | kDa | 1 | 20 | 5 | 4 |
| GN = MYO1E PE = 1 SV = 2 | | | | | | |
| Vacuolar protein sorting-associated protein VTA1 | 34 | kDa | 1 | 0 | 0 | 2 |
| homolog OS = Homo sapiens OX = 9606 GN = VTA1 PE = 1 | | | | | | |
| SV = 1 | | | | | | |
| Very-long-chain (3R)-3-hydroxyacyl-CoA dehydratase 3 | 43 | kDa | 1 | 8 | 1 | 2 |
| OS = Homo sapiens GN = HACD3 PE = 1 SV = 2 | | | | | | |
| von Willebrand factor A domain-containing protein 1 | 47 | kDa | 1 | 7 | 0 | 0 |
| OS = Homo sapiens OX = 9606 GN = VWA1 PE = 1 SV = 1 | | | | | | |
| (Bos taurus) 63 kDa protein | 63 | kDa | 0 | 55 | 0 | 0 |
| 10 kDa heat shock protein, mitochondrial OS = Homo | 11 | kDa | 0 | 6 | 0 | 0 |
| sapiens GN = HSPE1 PE = 1 SV = 2 | | | | | | |
| 1-phosphatidylinositol 3-phosphate 5-kinase OS = Homo | 237 | kDa | 0 | 0 | 0 | 2 |
| sapiens OX = 9606 GN = PIKFYVE PE = 1 SV = 3 | | | | | | |
| 26S protease regulatory subunit 10B OS = Homo sapiens | 46 | kDa | 0 | 9 | 0 | 0 |
| GN = PSMC6 PE = 1 SV = 1 | | | | | | |
| 26S protease regulatory subunit 6A OS = Homo sapiens | 49 | kDa | 0 | 30 | 0 | 0 |
| GN = PSMC3 PE = 1 SV = 3 | | | | | | |
| 26S proteasome non-ATPase regulatory subunit 14 | 35 | kDa | 0 | 17 | 0 | 0 |
| OS = Homo sapiens GN = PSMD14 PE = 1 SV = 1 | | | | | | |
| 26S proteasome non-ATPase regulatory subunit 8 | 33 | kDa | 0 | 3 | 0 | 0 |
| OS = Homo sapiens GN = PSMD8 PE = 1 SV = 1 | | | | | | |
| 28S ribosomal protein S34, mitochondrial OS = Homo | 26 | kDa | 0 | 2 | 0 | 0 |
| sapiens GN = MRPS34 PE = 1 SV = 2 | | | | | | |
| 39S ribosomal protein L11, mitochondrial OS = Homo | 21 | kDa | 0 | 7 | 0 | 1 |
| sapiens GN = MRPL11 PE = 1 SV = 1 | | | | | | |
| 39S ribosomal protein L34, mitochondrial OS = Homo | 20 | kDa | 0 | 8 | 0 | 0 |
| sapiens GN = MRPL34 PE = 1 SV = 1 | | | | | | |
| 3-ketoacyl-CoA thiolase, mitochondrial OS = Homo | 42 | kDa | 0 | 0 | 0 | 2 |
| sapiens OX = 9606 GN = ACAA2 PE = 1 SV = 2 | | | | | | |
| 40S ribosomal protein S13 OS = Homo sapiens OX = 9606 | 17 | kDa | 0 | 85 | 19 | 19 |
| GN = RPS13 PE = 1 SV = 2 | | | | | | |
| 40S ribosomal protein S15a OS = Homo sapiens | 11 | kDa | 0 | 25 | 0 | 0 |
| GN = RPS15A PE = 1 SV = 1 | | | | | | |
| 40S ribosomal protein S18 OS = Homo sapiens | 18 | kDa | 0 | 51 | 0 | 0 |
| GN = RPS18 PE = 1 SV = 3 | | | | | | |
| 40S ribosomal protein S28 OS = Homo sapiens | 8 | kDa | 0 | 6 | 0 | 0 |
| GN = RPS28 PE = 1 SV = 1 | | | | | | |
| 40S ribosomal protein S29 OS = Homo sapiens OX = 9606 | 7 | kDa | 0 | 16 | 1 | 1 |
| GN = RPS29 PE = 1 SV = 2 | | | | | | |
| 40S ribosomal protein S3a OS = Homo sapiens | 30 | kDa | 0 | 53 | 0 | 0 |
| GN = RPS3A PE = 1 SV = 2 | | | | | | |
| 40S ribosomal protein S4, Y isoform 1 OS = Homo | 29 | kDa | 0 | 0 | 0 | 9 |
| sapiens OX = 9606 GN = RPS4Y1 PE = 1 SV = 2 | | | | | | |
| 40S ribosomal protein SA (Fragment) OS = Homo sapiens | 29 | kDa | 0 | 8 | 0 | 0 |
| GN = RPSA PE = 1 SV = 8 | | | | | | |
| 60S acidic ribosomal protein P2 OS = Homo sapiens | 12 | kDa | 0 | 50 | 0 | 0 |
| GN = RPLP2 PE = 1 SV = 1 | | | | | | |
| 60S ribosomal protein L13a OS = Homo sapiens | 24 | kDa | 0 | 33 | 0 | 0 |
| GN = RPL13A PE = 1 SV = 2 | | | | | | |
| 60S ribosomal protein L14 OS = Homo sapiens | 23 | kDa | 0 | 34 | 0 | 0 |
| GN = RPL14 PE = 1 SV = 4 | | | | | | |
| 60S ribosomal protein L18a OS = Homo sapiens | 21 | kDa | 0 | 31 | 0 | 0 |
| GN = RPL18A PE = 1 SV = 2 | | | | | | |
| 60S ribosomal protein L23a (Fragment) OS = Homo | 18 | kDa | 0 | 32 | 0 | 0 |
| sapiens GN = RPL23A PE = 1 SV = 1 | | | | | | |
| 60S ribosomal protein L29 OS = Homo sapiens | 18 | kDa | 0 | 6 | 0 | 0 |
| GN = RPL29 PE = 1 SV = 2 | | | | | | |
| 60S ribosomal protein L32 (Fragment) OS = Homo | 16 | kDa | 0 | 20 | 0 | 0 |
| sapiens GN = RPL32 PE = 1 SV = 1 | | | | | | |
| 60S ribosomal protein L34 OS = Homo sapiens OX = 9606 | 13 | kDa | 0 | 0 | 3 | 3 |
| GN = RPL34 PE = 1 SV = 3 | | | | | | |
| 60S ribosomal protein L35 OS = Homo sapiens | 15 | kDa | 0 | 24 | 0 | 0 |
| GN = RPL35 PE = 1 SV = 2 | | | | | | |
| 60S ribosomal protein L38 OS = Homo sapiens OX = 9606 | 8 | kDa | 0 | 0 | 5 | 4 |
| GN = RPL38 PE = 1 SV = 2 | | | | | | |
| 78 kDa glucose-regulated protein OS = Homo sapiens | 72 | kDa | 0 | 188 | 0 | 0 |
| GN = HSPA5 PE = 1 SV = 2 | | | | | | |
| A disintegrin and metalloproteinase with thrombospondin | 105 | kDa | 0 | 12 | 3 | 0 |
| motifs 1 OS = Homo sapiens OX = 9606 GN = ADAMTS1 | | | | | | |
| PE = 1 SV = 4 | | | | | | |
| Actin-related protein 10 OS = Homo sapiens | 46 | kDa | 0 | 3 | 0 | 0 |
| GN = ACTR10 PE = 1 SV = 1 | | | | | | |
| Actin-related protein 2/3 complex subunit 1B OS = Homo | 41 | kDa | 0 | 28 | 0 | 0 |
| sapiens GN = ARPC1B PE = 1 SV = 3 | | | | | | |
| ADP-ribosyl cyclase/cyclic ADP-ribose hydrolase 2 | 36 | kDa | 0 | 3 | 5 | 2 |
| OS = Homo sapiens OX = 9606 GN = BST1 PE = 1 SV = 2 | | | | | | |
| Aminopeptidase N OS = Homo sapiens GN = ANPEP | 110 | kDa | 0 | 194 | 0 | 0 |
| PE = 1 SV = 4 | | | | | | |
| Angiopoietin-related protein 4 OS = Homo sapiens | 45 | kDa | 0 | 1 | 3 | 0 |
| OX = 9606 GN = ANGPTL4 PE = 1 SV = 2 | | | | | | |
| Annexin A3 OS = Homo sapiens OX = 9606 GN = ANXA3 | 36 | kDa | 0 | 0 | 0 | 3 |
| PE = 1 SV = 3 | | | | | | |
| Annexin A4 OS = Homo sapiens OX = 9606 GN = ANXA4 | 36 | kDa | 0 | 0 | 0 | 2 |
| PE = 1 SV = 4 | | | | | | |
| AP-2 complex subunit sigma OS = Homo sapiens | 19 | kDa | 0 | 3 | 0 | 0 |
| GN = AP2S1 PE = 1 SV = 1 | | | | | | |
| Arf-GAP with Rho-GAP domain, ANK repeat and PH | 170 | kDa | 0 | 0 | 0 | 2 |
| domain-containing protein 3 OS = Homo sapiens | | | | | | |
| OX = 9606 GN = ARAP3 PE = 1 SV = 1 | | | | | | |
| ATP synthase subunit epsilon-like protein, mitochondrial | 6 | kDa | 0 | 3 | 0 | 0 |
| OS = Homo sapiens GN = ATP5EP2 PE = 3 SV = 1 | | | | | | |
| ATP synthase subunit g, mitochondrial OS = Homo | 11 | kDa | 0 | 6 | 0 | 0 |
| sapiens GN = ATP5L PE = 1 SV = 3 | | | | | | |
| ATPase ASNA1 OS = Homo sapiens GN = ASNA1 PE = 1 | 39 | kDa | 0 | 2 | 0 | 0 |
| SV = 2 | | | | | | |
| ATPase, H+ transporting, lysosomal accessory protein 1, | 32 | kDa | 0 | 7 | 0 | 0 |
| isoform CRA_c OS = Homo sapiens GN = ATP6AP1 PE = 1 | | | | | | |
| SV = 1 | | | | | | |
| ATP-dependent DNA helicase Q1 OS = Homo sapiens | 73 | kDa | 0 | 2 | 0 | 0 |
| GN = RECQL PE = 1 SV = 3 | | | | | | |
| Barrier-to-autointegration factor OS = Homo sapiens | 10 | kDa | 0 | 24 | 0 | 0 |
| GN = BANF1 PE = 1 SV = 1 | | | | | | |
| Basement membrane-specific heparan sulfate | 469 | kDa | 0 | 4914 | 0 | 0 |
| proteoglycan core protein OS = Homo sapiens | | | | | | |
| GN = HSPG2 PE = 1 SV = 4 | | | | | | |
| Beta-catenin-like protein 1 OS = Homo sapiens | 65 | kDa | 0 | 2 | 0 | 0 |
| GN = CTNNBL1 PE = 1 SV = 1 | | | | | | |
| Beta-galactosidase OS = Homo sapiens GN = GLB1 PE = 1 | 76 | kDa | 0 | 3 | 0 | 0 |
| SV = 2 | | | | | | |
| Biglycan OS = Homo sapiens OX = 9606 GN = BGN PE = 1 | 42 | kDa | 0 | 0 | 23 | 0 |
| SV = 2 | | | | | | |
| Bone morphogenetic protein 1 OS = Homo sapiens | 111 | kDa | 0 | 14 | 3 | 0 |
| OX = 9606 GN = BMP1 PE = 1 SV = 2 | | | | | | |
| Brain acid soluble protein 1 OS = Homo sapiens | 23 | kDa | 0 | 10 | 0 | 0 |
| GN = BASP1 PE = 1 SV = 2 | | | | | | |
| C-1-tetrahydrofolate synthase, cytoplasmic OS = Homo | 102 | kDa | 0 | 2 | 0 | 0 |
| sapiens GN = MTHFD1 PE = 1 SV = 3 | | | | | | |
| Calmodulin OS = Homo sapiens GN = CALM1 PE = 1 | 17 | kDa | 0 | 24 | 0 | 0 |
| SV = 2 | | | | | | |
| Calpain small subunit 1 OS = Homo sapiens | 34 | kDa | 0 | 34 | 0 | 0 |
| GN = CAPNS1 PE = 1 SV = 1 | | | | | | |
| Carboxypeptidase M OS = Homo sapiens GN = CPM PE = 1 | 51 | kDa | 0 | 5 | 0 | 0 |
| SV = 2 | | | | | | |
| Cardiomyopathy-associated protein 5 OS = Homo sapiens | 449 | kDa | 0 | 2 | 0 | 0 |
| GN = CMYA5 PE = 1 SV = 3 | | | | | | |
| Casein kinase II subunit alpha OS = Homo sapiens | 45 | kDa | 0 | 10 | 1 | 1 |
| OX = 9606 GN = CSNK2A1 PE = 1 SV = 1 | | | | | | |
| CD2-associated protein OS = Homo sapiens GN = CD2AP | 71 | kDa | 0 | 9 | 0 | 0 |
| PE = 1 SV = 1 | | | | | | |
| CD59 glycoprotein OS = Homo sapiens OX = 9606 | 14 | kDa | 0 | 0 | 3 | 0 |
| GN = CD59 PE = 1 SV = 1 | | | | | | |
| Chloride intracellular channel protein 4 OS = Homo | 29 | kDa | 0 | 2 | 0 | 0 |
| sapiens GN = CLIC4 PE = 1 SV = 4 | | | | | | |
| Chloride intracellular channel protein 6 OS = Homo | 73 | kDa | 0 | 2 | 0 | 0 |
| sapiens GN = CLIC6 PE = 2 SV = 3 | | | | | | |
| Chromobox protein homolog 1 (Fragment) OS = Homo | 19 | kDa | 0 | 5 | 0 | 0 |
| sapiens GN = CBX1 PE = 1 SV = 1 | | | | | | |
| Coiled-coil domain-containing protein 124 OS = Homo | 26 | kDa | 0 | 6 | 0 | 0 |
| sapiens GN = CCDC124 PE = 1 SV = 1 | | | | | | |
| Coiled-coil domain-containing protein 30 OS = Homo | 91 | kDa | 0 | 2 | 0 | 0 |
| sapiens GN = CCDC30 PE = 2 SV = 1 | | | | | | |
| Coiled-coil-helix-coiled-coil-helix domain-containing | 13 | kDa | 0 | 2 | 0 | 0 |
| protein 1 OS = Homo sapiens GN = CHCHD1 PE = 1 SV = 1 | | | | | | |
| Collagen alpha-1(II) chain OS = Homo sapiens OX = 9606 | 142 | kDa | 0 | 2 | 1 | 1 |
| GN = COL2A1 PE = 1 SV = 3 | | | | | | |
| Collagen alpha-1(VI) chain OS = Homo sapiens | 108 | kDa | 0 | 3 | 0 | 0 |
| GN = COL6A1 PE = 1 SV = 1 | | | | | | |
| Collagen alpha-1(X) chain OS = Homo sapiens | 66 | kDa | 0 | 3 | 0 | 0 |
| GN = COL10A1 PE = 1 SV = 2 | | | | | | |
| Collagen alpha-1(XXIII) chain OS = Homo sapiens | 52 | kDa | 0 | 2 | 0 | 0 |
| GN = COL23A1 PE = 1 SV = 1 | | | | | | |
| Collagen alpha-1(XXVII) chain OS = Homo sapiens | 187 | kDa | 0 | 1 | 0 | 2 |
| OX = 9606 GN = COL27A1 PE = 1 SV = 1 | | | | | | |
| Collagen alpha-2(IX) chain OS = Homo sapiens OX = 9606 | 65 | kDa | 0 | 0 | 0 | 0 |
| GN = COL9A2 PE = 1 SV = 2 | | | | | | |
| Collagen triple helix repeat-containing protein 1 | 26 | kDa | 0 | 3 | 1 | 1 |
| OS = Homo sapiens OX = 9606 GN = CTHRC1 PE = 1 SV = 1 | | | | | | |
| Connective tissue growth factor OS = Homo sapiens | 38 | kDa | 0 | 4 | 6 | 2 |
| OX = 9606 GN = CTGF PE = 1 SV = 2 | | | | | | |
| COP9 signalosome complex subunit 5 OS = Homo sapiens | 38 | kDa | 0 | 2 | 0 | 0 |
| GN = COPS5 PE = 1 SV = 4 | | | | | | |
| Coronin-1B OS = Homo sapiens GN = CORO1B PE = 1 | 54 | kDa | 0 | 9 | 0 | 0 |
| SV = 1 | | | | | | |
| Cullin-1 OS = Homo sapiens GN = CUL1 PE = 1 SV = 2 | 90 | kDa | 0 | 3 | 0 | 0 |
| Cytochrome c (Fragment) OS = Homo sapiens GN = CYCS | 11 | kDa | 0 | 3 | 0 | 0 |
| PE = 1 SV = 1 | | | | | | |
| Cytochrome c oxidase subunit 4 isoform 1, mitochondrial | 20 | kDa | 0 | 5 | 0 | 0 |
| OS = Homo sapiens GN = COX4I1 PE = 1 SV = 1 | | | | | | |
| Cytoplasmic aconitate hydratase OS = Homo sapiens | 98 | kDa | 0 | 0 | 0 | 2 |
| OX = 9606 GN = ACO1 PE = 1 SV = 3 | | | | | | |
| Cytosolic non-specific dipeptidase OS = Homo sapiens | 53 | kDa | 0 | 10 | 0 | 8 |
| OX = 9606 GN = CNDP2 PE = 1 SV = 2 | | | | | | |
| Death-associated protein kinase 3 OS = Homo sapiens | 53 | kDa | 0 | 6 | 0 | 0 |
| OX = 9606 GN = DAPK3 PE = 1 SV = 1 | | | | | | |
| Deoxyribose-phosphate aldolase OS = Homo sapiens | 35 | kDa | 0 | 0 | 0 | 3 |
| OX = 9606 GN = DERA PE = 1 SV = 2 | | | | | | |
| Desmoglein-1 OS = Homo sapiens GN = DSG1 PE = 1 | 114 | kDa | 0 | 6 | 0 | 0 |
| SV = 2 | | | | | | |
| DNA damage-binding protein 1 OS = Homo sapiens | 127 | kDa | 0 | 14 | 0 | 3 |
| OX = 9606 GN = DDB1 PE = 1 SV = 1 | | | | | | |
| DNA topoisomerase 1 OS = Homo sapiens GN = TOP1 | 91 | kDa | 0 | 10 | 0 | 0 |
| PE = 1 SV = 2 | | | | | | |
| DNA-directed RNA polymerase II subunit RPB1 | 217 | kDa | 0 | 11 | 0 | 0 |
| OS = Homo sapiens OX = 9606 GN = POLR2A PE = 1 SV = 2 | | | | | | |
| DNA-directed RNA polymerase II subunit RPB2 | 134 | kDa | 0 | 14 | 0 | 0 |
| OS = Homo sapiens GN = POLR2B PE = 1 SV = 1 | | | | | | |
| DNA-directed RNA polymerase II subunit RPB3 | 31 | kDa | 0 | 9 | 0 | 0 |
| OS = Homo sapiens GN = POLR2C PE = 1 SV = 2 | | | | | | |
| DNA-directed RNA polymerase II subunit RPB4 | 13 | kDa | 0 | 6 | 0 | 0 |
| OS = Homo sapiens GN = POLR2D PE = 1 SV = 1 | | | | | | |
| DNA-directed RNA polymerases I, II, and III subunit | 17 | kDa | 0 | 2 | 3 | 0 |
| RPABC3 OS = Homo sapiens OX = 9606 GN = POLR2H | | | | | | |
| PE = 1 SV = 4 | | | | | | |
| DnaJ homolog subfamily B member 11 OS = Homo | 41 | kDa | 0 | 0 | 0 | 4 |
| sapiens OX = 9606 GN = DNAJB11 PE = 1 SV = 1 | | | | | | |
| Dolichol-phosphate mannosyltransferase subunit 1 | 30 | kDa | 0 | 2 | 0 | 0 |
| OS = Homo sapiens GN = DPM1 PE = 1 SV = 1 | | | | | | |
| Dolichyl-diphosphooligosaccharide--protein | 94 | kDa | 0 | 2 | 0 | 0 |
| glycosyltransferase subunit STT3B OS = Homo sapiens | | | | | | |
| GN = STT3B PE = 1 SV = 1 | | | | | | |
| Doublecortin domain-containing protein 2 OS = Homo | 53 | kDa | 0 | 17 | 0 | 2 |
| sapiens OX = 9606 GN = DCDC2 PE = 1 SV = 2 | | | | | | |
| Double-stranded RNA-binding protein Staufen homolog | 63 | kDa | 0 | 15 | 4 | 4 |
| 1 OS = Homo sapiens OX = 9606 GN = STAU1 PE = 1 SV = 2 | | | | | | |
| Dynactin subunit 2 OS = Homo sapiens GN = DCTN2 | 44 | kDa | 0 | 6 | 0 | 2 |
| PE = 1 SV = 4 | | | | | | |
| Dynein heavy chain 10, axonemal OS = Homo sapiens | 515 | kDa | 0 | 1 | 0 | 2 |
| OX = 9606 GN = DNAH10 PE = 1 SV = 4 | | | | | | |
| EBNA1 binding protein 2, isoform CRA_d OS = Homo | 41 | kDa | 0 | 5 | 0 | 0 |
| sapiens GN = EBNA1BP2 PE = 1 SV = 1 | | | | | | |
| Ectonucleotide pyrophosphatase/phosphodiesterase | 105 | kDa | 0 | 0 | 0 | 2 |
| family member 1 OS = Homo sapiens OX = 9606 | | | | | | |
| GN = ENPP1 PE = 1 SV = 2 | | | | | | |
| EH domain-containing protein 4 OS = Homo sapiens | 61 | kDa | 0 | 5 | 0 | 3 |
| OX = 9606 GN = EHD4 PE = 1 SV = 1 | | | | | | |
| Elongation factor Tu, mitochondrial OS = Homo sapiens | 50 | kDa | 0 | 9 | 0 | 0 |
| GN = TUFM PE = 1 SV = 2 | | | | | | |
| Emerin OS = Homo sapiens GN = EMD PE = 1 SV = 1 | 29 | kDa | 0 | 6 | 0 | 0 |
| Epoxide hydrolase 1 OS = Homo sapiens OX = 9606 | 53 | kDa | 0 | 0 | 0 | 3 |
| GN = EPHX1 PE = 1 SV = 1 | | | | | | |
| ER lumen protein-retaining receptor 3 OS = Homo sapiens | 25 | kDa | 0 | 5 | 0 | 0 |
| GN = KDELR3 PE = 2 SV = 1 | | | | | | |
| Eukaryotic translation initiation factor 3 subunit E | 52 | kDa | 0 | 8 | 0 | 0 |
| OS = Homo sapiens GN = EIF3E PE = 1 SV = 1 | | | | | | |
| Eukaryotic translation initiation factor 3 subunit F | 38 | kDa | 0 | 14 | 0 | 0 |
| OS = Homo sapiens GN = EIF3F PE = 1 SV = 1 | | | | | | |
| Eukaryotic translation initiation factor 3 subunit H | 40 | kDa | 0 | 5 | 0 | 0 |
| OS = Homo sapiens GN = EIF3H PE = 1 SV = 1 | | | | | | |
| FACT complex subunit SPT16 OS = Homo sapiens | 120 | kDa | 0 | 7 | 0 | 0 |
| GN = SUPT16H PE = 1 SV = 1 | | | | | | |
| Far upstream element-binding protein 1 OS = Homo | 68 | kDa | 0 | 3 | 0 | 1 |
| sapiens OX = 9606 GN = FUBP1 PE = 1 SV = 3 | | | | | | |
| Fascin OS = Homo sapiens GN = FSCN1 PE = 1 SV = 3 | 55 | kDa | 0 | 12 | 0 | 0 |
| Fibrous sheath-interacting protein 2 OS = Homo sapiens | 781 | kDa | 0 | 0 | 2 | 1 |
| OX = 9606 GN = FSIP2 PE = 2 SV = 4 | | | | | | |
| Filaggrin OS = Homo sapiens GN = FLG PE = 1 SV = 3 | 435 | kDa | 0 | 3 | 0 | 0 |
| Filaggrin-2 OS = Homo sapiens GN = FLG2 PE = 1 SV = 1 | 248 | kDa | 0 | 2 | 0 | 0 |
| Flotillin-2 OS = Homo sapiens GN = FLOT2 PE = 1 SV = 1 | 53 | kDa | 0 | 13 | 0 | 0 |
| Galectin-3 OS = Homo sapiens GN = LGALS3 PE = 1 SV = 5 | 26 | kDa | 0 | 39 | 0 | 0 |
| Galectin-3-binding protein OS = Homo sapiens OX = 9606 | 65 | kDa | 0 | 4 | 0 | 3 |
| GN = LGALS3BP PE = 1 SV = 1 | | | | | | |
| Galectin-8 OS = Homo sapiens OX = 9606 GN = LGALS8 | 36 | kDa | 0 | 5 | 2 | 0 |
| PE = 1 SV = 4 | | | | | | |
| Glutamate dehydrogenase 1, mitochondrial OS = Homo | 61 | kDa | 0 | 4 | 0 | 0 |
| sapiens OX = 9606 GN = GLUD1 PE = 1 SV = 2 | | | | | | |
| Glutathione peroxidase 1 OS = Homo sapiens GN = GPX1 | 22 | kDa | 0 | 3 | 0 | 2 |
| PE = 1 SV = 4 | | | | | | |
| Glycylpeptide N-tetradecanoyltransferase 2 OS = Homo | 57 | kDa | 0 | 2 | 0 | 0 |
| sapiens GN = NMT2 PE = 1 SV = 1 | | | | | | |
| Glypican-6 OS = Homo sapiens GN = GPC6 PE = 1 SV = 1 | 63 | kDa | 0 | 14 | 0 | 0 |
| Golgi-associated plant pathogenesis-related protein 1 | 17 | kDa | 0 | 20 | 0 | 0 |
| OS = Homo sapiens GN = GLIPR2 PE = 1 SV = 3 | | | | | | |
| Gremlin-1 OS = Homo sapiens OX = 9606 GN = GREM1 | 21 | kDa | 0 | 0 | 3 | 0 |
| PE = 1 SV = 1 | | | | | | |
| Guanine nucleotide-binding protein G(I)/G(S)/G(O) | 8 | kDa | 0 | 6 | 0 | 0 |
| subunit gamma-12 OS = Homo sapiens GN = GNG12 PE = 1 | | | | | | |
| SV = 3 | | | | | | |
| Guanine nucleotide-binding protein G(k) subunit alpha | 41 | kDa | 0 | 17 | 0 | 0 |
| OS = Homo sapiens GN = GNAI3 PE = 1 SV = 3 | | | | | | |
| Hamartin OS = Homo sapiens GN = TSC1 PE = 1 SV = 2 | 130 | kDa | 0 | 0 | 0 | 2 |
| Heat shock-related 70 kDa protein 2 OS = Homo sapiens | 70 | kDa | 0 | 0 | 0 | 24 |
| OX = 9606 GN = HSPA2 PE = 1 SV = 1 | | | | | | |
| Hemicentin-2 OS = Homo sapiens OX = 9606 | 542 | kDa | 0 | 0 | 0 | 0 |
| GN = HMCN2 PE = 2 SV = 3 | | | | | | |
| Heparan sulfate glucosamine 3-O-sulfotransferase 6 | 37 | kDa | 0 | 3 | 0 | 0 |
| OS = Homo sapiens GN = HS3ST6 PE = 1 SV = 2 | | | | | | |
| Heterogeneous nuclear ribonucleoprotein A0 OS = Homo | 31 | kDa | 0 | 15 | 0 | 0 |
| sapiens GN = HNRNPA0 PE = 1 SV = 1 | | | | | | |
| Heterogeneous nuclear ribonucleoprotein H2 OS = Homo | 49 | kDa | 0 | 14 | 0 | 0 |
| sapiens GN = HNRNPH2 PE = 1 SV = 1 | | | | | | |
| Homeobox protein Hox-B3 OS = Homo sapiens OX = 9606 | 44 | kDa | 0 | 5 | 0 | 0 |
| GN = HOXB3 PE = 2 SV = 2 | | | | | | |
| Hornerin OS = Homo sapiens GN = HRNR PE = 1 SV = 2 | 282 | kDa | 0 | 15 | 0 | 0 |
| Hsc70-interacting protein (Fragment) OS = Homo sapiens | 16 | kDa | 0 | 2 | 0 | 0 |
| GN = ST13 PE = 1 SV = 1 | | | | | | |
| Hyaluronan and proteoglycan link protein 1 OS = Homo | 40 | kDa | 0 | 0 | 0 | 0 |
| sapiens OX = 9606 GN = HAPLN1 PE = 2 SV = 2 | | | | | | |
| Hyaluronan and proteoglycan link protein 3 OS = Homo | 41 | kDa | 0 | 6 | 0 | 0 |
| sapiens GN = HAPLN3 PE = 2 SV = 1 | | | | | | |
| Intercellular adhesion molecule 1 OS = Homo sapiens | 58 | kDa | 0 | 16 | 0 | 0 |
| GN = ICAM1 PE = 1 SV = 2 | | | | | | |
| Interleukin enhancer-binding factor 2 OS = Homo sapiens | 39 | kDa | 0 | 29 | 0 | 0 |
| GN = ILF2 PE = 1 SV = 1 | | | | | | |
| Isocitrate dehydrogenase [NADP] cytoplasmic | 47 | kDa | 0 | 7 | 0 | 0 |
| OS = Homo sapiens GN = IDH1 PE = 1 SV = 2 | | | | | | |
| Isoform 1 of Gamma-adducin OS = Homo sapiens | 76 | kDa | 0 | 7 | 0 | 1 |
| OX = 9606 GN = ADD3 | | | | | | |
| Isoform 1 of Polypyrimidine tract-binding protein 3 | 57 | kDa | 0 | 5 | 0 | 3 |
| OS = Homo sapiens OX = 9606 GN = PTBP3 | | | | | | |
| Isoform 1 of Synaptic functional regulator FMR1 | 67 | kDa | 0 | 5 | 0 | 1 |
| OS = Homo sapiens OX = 9606 GN = FMR1 | | | | | | |
| Isoform 2 of 2,4-dienoyl-CoA reductase, mitochondrial | 35 | kDa | 0 | 8 | 0 | 1 |
| OS = Homo sapiens OX = 9606 GN = DECR1 | | | | | | |
| Isoform 2 of A-kinase anchor protein 13 OS = Homo | 308 | kDa | 0 | 2 | 0 | 0 |
| sapiens OX = 9606 GN = AKAP13 | | | | | | |
| Isoform 2 of Ankyrin repeat domain-containing protein | 274 | kDa | 0 | 0 | 0 | 0 |
| 17 OS = Homo sapiens OX = 9606 GN = ANKRD17 | | | | | | |
| Isoform 2 of AP-2 complex subunit mu OS = Homo | 49 | kDa | 0 | 9 | 0 | 1 |
| sapiens OX = 9606 GN = AP2M1 | | | | | | |
| Isoform 2 of Bcl-2-associated transcription factor 1 | 106 | kDa | 0 | 4 | 0 | 0 |
| OS = Homo sapiens OX = 9606 GN = BCLAF1 | | | | | | |
| Isoform 2 of Cadherin-2 OS = Homo sapiens OX = 9606 | 97 | kDa | 0 | 7 | 0 | 2 |
| GN = CDH2 | | | | | | |
| Isoform 2 of Calponin-1 OS = Homo sapiens OX = 9606 | 31 | kDa | 0 | 1 | 5 | 8 |
| GN = CNN1 | | | | | | |
| Isoform 2 of Chromodomain Y-like protein OS = Homo | 61 | kDa | 0 | 4 | 0 | 0 |
| sapiens GN = CDYL | | | | | | |
| Isoform 2 of Collagen alpha-1(VII) chain OS = Homo | 292 | kDa | 0 | 70 | 2 | 1 |
| sapiens OX = 9606 GN = COL7A1 | | | | | | |
| Isoform 2 of Cyclin-Y OS = Homo sapiens GN = CCNY | 37 | kDa | 0 | 3 | 0 | 0 |
| Isoform 2 of Cysteine-rich protein 2 OS = Homo sapiens | 30 | kDa | 0 | 5 | 0 | 0 |
| OX = 9606 GN = CRIP2 | | | | | | |
| Isoform 2 of DnaJ homolog subfamily C member 10 | 86 | kDa | 0 | 4 | 0 | 0 |
| OS = Homo sapiens GN = DNAJC10 | | | | | | |
| Isoform 2 of Fibroblast growth factor 2 OS = Homo | 23 | kDa | 0 | 13 | 3 | 1 |
| sapiens OX = 9606 GN = FGF2 | | | | | | |
| Isoform 2 of Fibulin-2 OS = Homo sapiens OX = 9606 | 132 | kDa | 0 | 6 | 0 | 0 |
| GN = FBLN2 | | | | | | |
| Isoform 2 of GDNF family receptor alpha-1 OS = Homo | 51 | kDa | 0 | 8 | 0 | 0 |
| sapiens GN = GFRA1 | | | | | | |
| Isoform 2 of Glycogen phosphorylase, liver form | 93 | kDa | 0 | 2 | 0 | 1 |
| OS = Homo sapiens OX = 9606 GN = PYGL | | | | | | |
| Isoform 2 of Golgin subfamily A member 5 OS = Homo | 78 | kDa | 0 | 0 | 0 | 0 |
| sapiens OX = 9606 GN = GOLGA5 | | | | | | |
| Isoform 2 of H/ACA ribonucleoprotein complex subunit | 21 | kDa | 0 | 1 | 1 | 2 |
| 1 OS = Homo sapiens OX = 9606 GN = GAR1 | | | | | | |
| Isoform 2 of Helicase SRCAP OS = Homo sapiens | 337 | kDa | 0 | 2 | 0 | 0 |
| OX = 9606 GN = SRCAP | | | | | | |
| Isoform 2 of Histone-binding protein RBBP4 OS = Homo | 48 | kDa | 0 | 6 | 0 | 0 |
| sapiens OX = 9606 GN = RBBP4 | | | | | | |
| Isoform 2 of Histone-lysine N-methyltransferase, H3 | 267 | kDa | 0 | 6 | 0 | 1 |
| lysine-36 and H4 lysine-20 specific OS = Homo sapiens | | | | | | |
| OX = 9606 GN = NSD1 | | | | | | |
| Isoform 2 of Insulin-like growth factor-binding protein 7 | 29 | kDa | 0 | 54 | 13 | 7 |
| OS = Homo sapiens OX = 9606 GN = IGFBP7 | | | | | | |
| Isoform 2 of Interferon-inducible double-stranded RNA- | 33 | kDa | 0 | 4 | 1 | 1 |
| dependent protein kinase activator A OS = Homo sapiens | | | | | | |
| OX = 9606 GN = PRKRA | | | | | | |
| Isoform 2 of KH domain-containing, RNA-binding, | 30 | kDa | 0 | 8 | 0 | 0 |
| signal transduction-associated protein 3 OS = Homo | | | | | | |
| sapiens GN = KHDRBS3 | | | | | | |
| Isoform 2 of Lactadherin OS = Homo sapiens | 35 | kDa | 0 | 25 | 2 | 1 |
| GN = MFGE8 | | | | | | |
| Isoform 2 of L-amino-acid oxidase OS = Homo sapiens | 65 | kDa | 0 | 2 | 0 | 1 |
| OX = 9606 GN = IL4I1 | | | | | | |
| Isoform 2 of Macrophage-capping protein OS = Homo | 37 | kDa | 0 | 3 | 0 | 0 |
| sapiens GN = CAPG | | | | | | |
| Isoform 2 of Matrilin-2 OS = Homo sapiens OX = 9606 | 105 | kDa | 0 | 37 | 8 | 5 |
| GN = MATN2 | | | | | | |
| Isoform 2 of Mesoderm-specific transcript homolog | 38 | kDa | 0 | 0 | 0 | 4 |
| protein OS = Homo sapiens OX = 9606 GN = MEST | | | | | | |
| Isoform 2 of Microtubule-associated protein 1A | 306 | kDa | 0 | 4 | 2 | 3 |
| OS = Homo sapiens OX = 9606 GN = MAP1A | | | | | | |
| Isoform 2 of Midkine OS = Homo sapiens OX = 9606 | 10 | kDa | 0 | 4 | 2 | 2 |
| GN = MDK | | | | | | |
| Isoform 2 of Monoacylglycerol lipase ABHD12 | 46 | kDa | 0 | 2 | 0 | 0 |
| OS = Homo sapiens GN = ABHD12 | | | | | | |
| Isoform 2 of Multidrug resistance protein 1 OS = Homo | 134 | kDa | 0 | 1 | 0 | 2 |
| sapiens OX = 9606 GN = ABCB1 | | | | | | |
| Isoform 2 of Myosin-11 OS = Homo sapiens GN = MYH11 | 228 | kDa | 0 | 161 | 37 | 0 |
| Isoform 2 of Myosin-14 OS = Homo sapiens OX = 9606 | 232 | kDa | 0 | 141 | 26 | 26 |
| GN = MYH14 | | | | | | |
| Isoform 2 of NADH dehydrogenase [ubiquinone] 1 beta | 14 | kDa | 0 | 1 | 0 | 2 |
| subcomplex subunit 4 OS = Homo sapiens OX = 9606 | | | | | | |
| GN = NDUFB4 | | | | | | |
| Isoform 2 of Nuclear receptor corepressor 1 OS = Homo | 259 | kDa | 0 | 0 | 2 | 0 |
| sapiens OX = 9606 GN = NCOR1 | | | | | | |
| Isoform 2 of Periostin OS = Homo sapiens OX = 9606 | 87 | kDa | 0 | 0 | 5 | 5 |
| GN = POSTN | | | | | | |
| Isoform 2 of Pescadillo homolog OS = Homo sapiens | 67 | kDa | 0 | 5 | 1 | 0 |
| OX = 9606 GN = PES1 | | | | | | |
| Isoform 2 of Platelet-derived growth factor subunit B | 26 | kDa | 0 | 1 | 3 | 1 |
| OS = Homo sapiens OX = 9606 GN = PDGFB | | | | | | |
| Isoform 2 of Protein ELYS OS = Homo sapiens OX = 9606 | 256 | kDa | 0 | 0 | 2 | 0 |
| GN = AHCTF1 | | | | | | |
| Isoform 2 of Ran-binding protein 3 OS = Homo sapiens | 60 | kDa | 0 | 5 | 0 | 0 |
| OX = 9606 GN = RANBP3 | | | | | | |
| Isoform 2 of Regulator of chromosome condensation | 48 | kDa | 0 | 16 | 0 | 0 |
| OS = Homo sapiens OX = 9606 GN = RCC1 | | | | | | |
| Isoform 2 of Retinol-binding protein 1 OS = Homo | 17 | kDa | 0 | 0 | 0 | 2 |
| sapiens OX = 9606 GN = RBP1 | | | | | | |
| Isoform 2 of RNA-binding protein 39 OS = Homo sapiens | 59 | kDa | 0 | 0 | 0 | 2 |
| GN = RBM39 | | | | | | |
| Isoform 2 of RNA-binding protein 8A OS = Homo sapiens | 20 | kDa | 0 | 5 | 0 | 1 |
| OX = 9606 GN = RBM8A | | | | | | |
| Isoform 2 of Ryanodine receptor 1 OS = Homo sapiens | 565 | kDa | 0 | 1 | 0 | 2 |
| OX = 9606 GN = RYR1 | | | | | | |
| Isoform 2 of SCO-spondin OS = Homo sapiens OX = 9606 | 139 | kDa | 0 | 3 | 0 | 0 |
| GN = SSPO | | | | | | |
| Isoform 2 of Semaphorin-7A OS = Homo sapiens | 73 | kDa | 0 | 3 | 1 | 3 |
| OX = 9606 GN = SEMA7A | | | | | | |
| Isoform 2 of Septin-8 OS = Homo sapiens OX = 9606 | 50 | kDa | 0 | 12 | 0 | 3 |
| GN = SEPT8 | | | | | | |
| Isoform 2 of Serine/threonine-protein phosphatase | 28 | kDa | 0 | 5 | 0 | 1 |
| PGAM5, mitochondrial OS = Homo sapiens OX = 9606 | | | | | | |
| GN = PGAM5 | | | | | | |
| Isoform 2 of SH3 domain-containing kinase-binding | 69 | kDa | 0 | 2 | 0 | 2 |
| protein 1 OS = Homo sapiens OX = 9606 GN = SH3KBP1 | | | | | | |
| Isoform 2 of Signal recognition particle receptor subunit | 67 | kDa | 0 | 3 | 0 | 1 |
| alpha OS = Homo sapiens OX = 9606 GN = SRPRA | | | | | | |
| Isoform 2 of Signal-induced proliferation-associated 1- | 197 | kDa | 0 | 0 | 0 | 0 |
| like protein 1 OS = Homo sapiens OX = 9606 | | | | | | |
| GN = SIPA1L1 | | | | | | |
| Isoform 2 of Sorting nexin-27 OS = Homo sapiens | 60 | kDa | 0 | 4 | 0 | 0 |
| GN = SNX27 | | | | | | |
| Isoform 2 of Spectrin beta chain, erythrocytic OS = Homo | 268 | kDa | 0 | 8 | 0 | 2 |
| sapiens OX = 9606 GN = SPTB | | | | | | |
| Isoform 2 of Testis-expressed protein 10 OS = Homo | 104 | kDa | 0 | 2 | 0 | 0 |
| sapiens OX = 9606 GN = TEX10 | | | | | | |
| Isoform 2 of Tissue factor pathway inhibitor 2 OS = Homo | 26 | kDa | 0 | 9 | 6 | 0 |
| sapiens OX = 9606 GN = TFPI2 | | | | | | |
| Isoform 2 of Tyrosine-protein kinase BAZ1B OS = Homo | 170 | kDa | 0 | 4 | 0 | 0 |
| sapiens OX = 9606 GN = BAZ1B | | | | | | |
| Isoform 2 of UDP-glucuronosyltransferase 1-6 | 30 | kDa | 0 | 4 | 0 | 0 |
| OS = Homo sapiens OX = 9606 GN = UGT1A6 | | | | | | |
| Isoform 2 of Vesicle-associated membrane protein- | 33 | kDa | 0 | 3 | 0 | 1 |
| associated protein A OS = Homo sapiens OX = 9606 | | | | | | |
| GN = VAPA | | | | | | |
| Isoform 2 of Voltage-dependent calcium channel subunit | 123 | kDa | 0 | 2 | 1 | 0 |
| alpha-2/delta-1 OS = Homo sapiens OX = 9606 | | | | | | |
| GN = CACNA2D1 | | | | | | |
| Isoform 2 of Y-box-binding protein 3 OS = Homo sapiens | 32 | kDa | 0 | 15 | 0 | 1 |
| OX = 9606 GN = YBX3 | | | | | | |
| Isoform 2 of Zinc finger homeobox protein 4 OS = Homo | 397 | kDa | 0 | 2 | 0 | 0 |
| sapiens OX = 9606 GN = ZFHX4 | | | | | | |
| Isoform 3 of 1-phosphatidylinositol 4,5-bisphosphate | 136 | kDa | 0 | 3 | 0 | 0 |
| phosphodiesterase beta-4 OS = Homo sapiens OX = 9606 | | | | | | |
| GN = PLCB4 | | | | | | |
| Isoform 3 of Alpha-adducin OS = Homo sapiens | 84 | kDa | 0 | 2 | 0 | 0 |
| GN = ADD1 | | | | | | |
| Isoform 3 of Cytoskeleton-associated protein 5 | 226 | kDa | 0 | 3 | 0 | 0 |
| OS = Homo sapiens OX = 9606 GN = CKAP5 | | | | | | |
| Isoform 3 of DnaJ homolog subfamily C member 11 | 57 | kDa | 0 | 2 | 0 | 1 |
| OS = Homo sapiens GN = DNAJC11 | | | | | | |
| Isoform 3 of E3 ubiquitin-protein ligase CHFR | 69 | kDa | 0 | 0 | 0 | 0 |
| OS = Homo sapiens OX = 9606 GN = CHFR | | | | | | |
| Isoform 3 of H/ACA ribonucleoprotein complex subunit | 48 | kDa | 0 | 13 | 1 | 1 |
| DKC1 OS = Homo sapiens OX = 9606 GN = DKC1 | | | | | | |
| Isoform 3 of Histone-lysine N-methyltransferase 2D | 594 | kDa | 0 | 1 | 0 | 1 |
| OS = Homo sapiens OX = 9606 GN = KMT2D | | | | | | |
| Isoform 3 of Latent-transforming growth factor beta- | 169 | kDa | 0 | 34 | 0 | 0 |
| binding protein 4 OS = Homo sapiens OX = 9606 | | | | | | |
| GN = LTBP4 | | | | | | |
| Isoform 3 of Malate dehydrogenase, cytoplasmic | 39 | kDa | 0 | 3 | 0 | 0 |
| OS = Homo sapiens OX = 9606 GN = MDH1 | | | | | | |
| Isoform 3 of Putative oxidoreductase GLYR1 OS = Homo | 60 | kDa | 0 | 4 | 0 | 0 |
| sapiens OX = 9606 GN = GLYR1 | | | | | | |
| Isoform 3 of Scaffold attachment factor B1 OS = Homo | 103 | kDa | 0 | 3 | 0 | 0 |
| sapiens GN = SAFB | | | | | | |
| Isoform 3 of Torsin-1A-interacting protein 1 OS = Homo | 66 | kDa | 0 | 5 | 0 | 0 |
| sapiens GN = TOR1AIP1 | | | | | | |
| Isoform 4 of CD109 antigen OS = Homo sapiens | 160 | kDa | 0 | 18 | 0 | 0 |
| GN = CD109 | | | | | | |
| Isoform 4 of FYVE and coiled-coil domain-containing | 169 | kDa | 0 | 0 | 0 | 0 |
| protein 1 OS = Homo sapiens OX = 9606 GN = FYCO1 | | | | | | |
| Isoform 4 of IQ domain-containing protein N OS = Homo | 147 | kDa | 0 | 0 | 0 | 3 |
| sapiens OX = 9606 GN = IQCN | | | | | | |
| Isoform 4 of Kinesin-like protein KIF24 OS = Homo | 129 | kDa | 0 | 2 | 0 | 0 |
| sapiens OX = 9606 GN = KIF24 | | | | | | |
| Isoform 4 of Latent-transforming growth factor beta- | 187 | kDa | 0 | 1 | 4 | 1 |
| binding protein 1 OS = Homo sapiens OX = 9606 | | | | | | |
| GN = LTBP1 | | | | | | |
| Isoform 4 of Protein diaphanous homolog 3 OS = Homo | 136 | kDa | 0 | 2 | 0 | 0 |
| sapiens GN = DIAPH3 | | | | | | |
| Isoform 5 of E1A-binding protein p400 OS = Homo | 340 | kDa | 0 | 1 | 0 | 0 |
| sapiens OX = 9606 GN = EP400 | | | | | | |
| Isoform 5 of Immunoglobulin-like and fibronectin type | 384 | kDa | 0 | 3 | 0 | 2 |
| III domain-containing protein 1 OS = Homo sapiens | | | | | | |
| OX = 9606 GN = IGFN1 | | | | | | |
| Isoform 5 of LIM domain only protein 7 OS = Homo | 158 | kDa | 0 | 8 | 0 | 0 |
| sapiens GN = LMO7 | | | | | | |
| Isoform 5 of Papilin OS = Homo sapiens OX = 9606 | 136 | kDa | 0 | 17 | 1 | 1 |
| GN = PAPLN | | | | | | |
| Isoform 6 of Treacle protein OS = Homo sapiens | 148 | kDa | 0 | 2 | 0 | 0 |
| OX = 9606 GN = TCOF1 | | | | | | |
| Isoform B of Collagen alpha-1(XI) chain OS = Homo | 182 | kDa | 0 | 0 | 1 | 2 |
| sapiens OX = 9606 GN = COL11A1 | | | | | | |
| Isoform B of Collagen alpha-6(IV) chain OS = Homo | 164 | kDa | 0 | 5 | 2 | 0 |
| sapiens OX = 9606 GN = COL4A6 | | | | | | |
| Isoform B of DnaJ homolog subfamily B member 6 | 27 | kDa | 0 | 0 | 1 | 2 |
| OS = Homo sapiens OX = 9606 GN = DNAJB6 | | | | | | |
| Isoform B of Methyl-CpG-binding protein 2 OS = Homo | 53 | kDa | 0 | 11 | 1 | 0 |
| sapiens OX = 9606 GN = MECP2 | | | | | | |
| Isoform B of Ras-related C3 botulinum toxin substrate 1 | 23 | kDa | 0 | 3 | 0 | 0 |
| OS = Homo sapiens OX = 9606 GN = RAC1 | | | | | | |
| Isoform B of Transforming growth factor beta-2 | 51 | kDa | 0 | 5 | 1 | 2 |
| proprotein OS = Homo sapiens OX = 9606 GN = TGFB2 | | | | | | |
| Isoform Beta-3B of Integrin beta-3 OS = Homo sapiens | 86 | kDa | 0 | 4 | 0 | 2 |
| OX = 9606 GN = ITGB3 | | | | | | |
| Isoform C of Fibulin-1 OS = Homo sapiens OX = 9606 | 74 | kDa | 0 | 13 | 0 | 8 |
| GN = FBLN1 | | | | | | |
| Isoform Long of Proteasome subunit alpha type-1 | 30 | kDa | 0 | 2 | 0 | 2 |
| OS = Homo sapiens OX = 9606 GN = PSMA1 | | | | | | |
| Isoform Non-brain of Clathrin light chain A OS = Homo | 24 | kDa | 0 | 17 | 0 | 2 |
| sapiens OX = 9606 GN = CLTA | | | | | | |
| Isoform Short of Laminin subunit gamma-2 OS = Homo | 122 | kDa | 0 | 5 | 0 | 1 |
| sapiens OX = 9606 GN = LAMC2 | | | | | | |
| Keratin, type I cytoskeletal 14 OS = Homo sapiens | 52 | kDa | 0 | 94 | 0 | 18 |
| OX = 9606 GN = KRT14 PE = 1 SV = 4 | | | | | | |
| Keratin, type I cytoskeletal 16 OS = Homo sapiens | 51 | kDa | 0 | 99 | 0 | 0 |
| GN = KRT16 PE = 1 SV = 4 | | | | | | |
| Keratin, type I cytoskeletal 19 OS = Homo sapiens | 44 | kDa | 0 | 753 | 0 | 0 |
| GN = KRT19 PE = 1 SV = 4 | | | | | | |
| Keratin, type II cuticular Hb5 OS = Homo sapiens | 56 | kDa | 0 | 3 | 0 | 0 |
| GN = KRT85 PE = 1 SV = 1 | | | | | | |
| Keratin, type II cytoskeletal 1 OS = Homo sapiens | 66 | kDa | 0 | 497 | 0 | 0 |
| GN = KRT1 PE = 1 SV = 6 | | | | | | |
| Keratin, type II cytoskeletal 5 OS = Homo sapiens | 62 | kDa | 0 | 93 | 0 | 0 |
| GN = KRT5 PE = 1 SV = 3 | | | | | | |
| Keratin, type II cytoskeletal 6B OS = Homo sapiens | 60 | kDa | 0 | 104 | 18 | 0 |
| OX = 9606 GN = KRT6B PE = 1 SV = 5 | | | | | | |
| Keratin, type II cytoskeletal 7 OS = Homo sapiens | 51 | kDa | 0 | 0 | 0 | 135 |
| OX = 9606 GN = KRT7 PE = 1 SV = 5 | | | | | | |
| Kinesin-like protein KIFC2 OS = Homo sapiens OX = 9606 | 90 | kDa | 0 | 2 | 0 | 1 |
| GN = KIFC2 PE = 2 SV = 1 | | | | | | |
| Ladinin-1 OS = Homo sapiens OX = 9606 GN = LAD1 | 57 | kDa | 0 | 0 | 0 | 6 |
| PE = 1 SV = 2 | | | | | | |
| Laminin subunit beta-3 OS = Homo sapiens GN = LAMB3 | 130 | kDa | 0 | 10 | 0 | 0 |
| PE = 1 SV = 1 | | | | | | |
| Latent-transforming growth factor beta-binding protein 2 | 190 | kDa | 0 | 23 | 0 | 0 |
| OS = Homo sapiens GN = LTBP2 PE = 1 SV = 1 | | | | | | |
| Leucine zipper protein 1 OS = Homo sapiens OX = 9606 | 120 | kDa | 0 | 5 | 0 | 0 |
| GN = LUZP1 PE = 1 SV = 2 | | | | | | |
| Lipoamide acyltransferase component of branched-chain | 53 | kDa | 0 | 6 | 4 | 8 |
| alpha-keto acid dehydrogenase complex, mitochondrial | | | | | | |
| OS = Homo sapiens OX = 9606 GN = DBT PE = 1 SV = 3 | | | | | | |
| Low-density lipoprotein receptor-related protein 1B | 515 | kDa | 0 | 2 | 0 | 0 |
| OS = Homo sapiens GN = LRP1B PE = 1 SV = 2 | | | | | | |
| Low-density lipoprotein receptor-related protein 2 | 522 | kDa | 0 | 0 | 0 | 2 |
| OS = Homo sapiens OX = 9606 GN = LRP2 PE = 1 SV = 3 | | | | | | |
| Lysyl oxidase homolog 1 OS = Homo sapiens OX = 9606 | 63 | kDa | 0 | 24 | 1 | 0 |
| GN = LOXL1 PE = 1 SV = 2 | | | | | | |
| Magnesium transporter protein 1 OS = Homo sapiens | 42 | kDa | 0 | 4 | 1 | 1 |
| GN = MAGT1 PE = 1 SV = 1 | | | | | | |
| MARCKS-related protein OS = Homo sapiens | 20 | kDa | 0 | 4 | 0 | 0 |
| GN = MARCKSL1 PE = 1 SV = 2 | | | | | | |
| Metastasis-associated protein MTA2 OS = Homo sapiens | 75 | kDa | 0 | 5 | 0 | 0 |
| GN = MTA2 PE = 1 SV = 1 | | | | | | |
| Microsomal glutathione S-transferase 1 OS = Homo | 18 | kDa | 0 | 0 | 0 | 3 |
| sapiens OX = 9606 GN = MGST1 PE = 1 SV = 1 | | | | | | |
| Mitochondrial GTPase 1 OS = Homo sapiens OX = 9606 | 37 | kDa | 0 | 0 | 0 | 2 |
| PE = 3 SV = 1 | | | | | | |
| MKI67 FHA domain-interacting nucleolar | 20 | kDa | 0 | 6 | 0 | 0 |
| phosphoprotein (Fragment) OS = Homo sapiens | | | | | | |
| GN = NIFK PE = 1 SV = 1 | | | | | | |
| Mucin-16 OS = Homo sapiens OX = 9606 GN = MUC16 | 1519 | kDa | 0 | 0 | 2 | 4 |
| PE = 1 SV = 3 | | | | | | |
| Myelin expression factor 2 OS = Homo sapiens OX = 9606 | 64 | kDa | 0 | 2 | 0 | 0 |
| GN = MYEF2 PE = 1 SV = 3 | | | | | | |
| Myosin light chain 6B OS = Homo sapiens OX = 9606 | 23 | kDa | 0 | 0 | 6 | 0 |
| GN = MYL6B PE = 1 SV = 1 | | | | | | |
| Myristoylated alanine-rich C-kinase substrate OS = Homo | 32 | kDa | 0 | 11 | 0 | 0 |
| sapiens GN = MARCKS PE = 1 SV = 4 | | | | | | |
| N-acylneuraminate cytidylyltransferase OS = Homo | 48 | kDa | 0 | 6 | 0 | 0 |
| sapiens GN = CMAS PE = 1 SV = 2 | | | | | | |
| NAD(P) transhydrogenase, mitochondrial OS = Homo | 100 | kDa | 0 | 3 | 0 | 0 |
| sapiens GN = NNT PE = 1 SV = 1 | | | | | | |
| Nestin OS = Homo sapiens GN = NES PE = 1 SV = 2 | 177 | kDa | 0 | 18 | 0 | 0 |
| Neurabin-2 OS = Homo sapiens GN = PPP1R9B PE = 1 | 89 | kDa | 0 | 3 | 0 | 0 |
| SV = 1 | | | | | | |
| Neurobeachin OS = Homo sapiens OX = 9606 GN = NBEA | 328 | kDa | 0 | 0 | 0 | 3 |
| PE = 1 SV = 3 | | | | | | |
| Nicotinate-nucleotide pyrophosphorylase [carboxylating] | 31 | kDa | 0 | 0 | 0 | 4 |
| OS = Homo sapiens OX = 9606 GN = QPRT PE = 1 SV = 3 | | | | | | |
| Non-syndromic hearing impairment protein 5 OS = Homo | 55 | kDa | 0 | 2 | 0 | 0 |
| sapiens GN = DFNA5 PE = 1 SV = 2 | | | | | | |
| Nuclear receptor-binding protein OS = Homo sapiens | 61 | kDa | 0 | 5 | 0 | 0 |
| GN = NRBP1 PE = 1 SV = 1 | | | | | | |
| Nucleolar complex protein 3 homolog OS = Homo sapiens | 93 | kDa | 0 | 5 | 0 | 0 |
| GN = NOC3L PE = 1 SV = 1 | | | | | | |
| Nucleolar complex protein 4 homolog OS = Homo sapiens | 58 | kDa | 0 | 5 | 0 | 0 |
| GN = NOC4L PE = 1 SV = 1 | | | | | | |
| Nucleolar protein 58 OS = Homo sapiens GN = NOP58 | 60 | kDa | 0 | 6 | 0 | 0 |
| PE = 1 SV = 1 | | | | | | |
| Nucleolar transcription factor 1 OS = Homo sapiens | 87 | kDa | 0 | 2 | 0 | 0 |
| GN = UBTF PE = 1 SV = 1 | | | | | | |
| Nucleoplasmin-3 OS = Homo sapiens GN = NPM3 PE = 1 | 19 | kDa | 0 | 3 | 0 | 0 |
| SV = 3 | | | | | | |
| Palladin OS = Homo sapiens OX = 9606 GN = PALLD | 151 | kDa | 0 | 7 | 0 | 6 |
| PE = 1 SV = 3 | | | | | | |
| PDZ and LIM domain protein 1 OS = Homo sapiens | 36 | kDa | 0 | 0 | 0 | 4 |
| OX = 9606 GN = PDLIM1 PE = 1 SV = 4 | | | | | | |
| PDZ and LIM domain protein 4 OS = Homo sapiens | 35 | kDa | 0 | 3 | 0 | 0 |
| GN = PDLIM4 PE = 1 SV = 2 | | | | | | |
| Pentraxin-related protein PTX3 OS = Homo sapiens | 42 | kDa | 0 | 23 | 0 | 0 |
| GN = PTX3 PE = 1 SV = 3 | | | | | | |
| Peptidyl-prolyl cis-trans isomerase B OS = Homo sapiens | 24 | kDa | 0 | 52 | 0 | 0 |
| GN = PPIB PE = 1 SV = 2 | | | | | | |
| Peptidyl-prolyl cis-trans isomerase FKBP10 OS = Homo | 64 | kDa | 0 | 3 | 0 | 0 |
| sapiens GN = FKBP10 PE = 1 SV = 1 | | | | | | |
| Peptidyl-prolyl cis-trans isomerase FKBP3 OS = Homo | 25 | kDa | 0 | 8 | 0 | 0 |
| sapiens GN = FKBP3 PE = 1 SV = 1 | | | | | | |
| Periaxin OS = Homo sapiens OX = 9606 GN = PRX PE = 1 | 155 | kDa | 0 | 0 | 0 | 2 |
| SV = 2 | | | | | | |
| Periodic tryptophan protein 1 homolog OS = Homo | 56 | kDa | 0 | 2 | 0 | 0 |
| sapiens OX = 9606 GN = PWP1 PE = 1 SV = 1 | | | | | | |
| Peroxiredoxin-1 (Fragment) OS = Homo sapiens | 19 | kDa | 0 | 13 | 0 | 0 |
| GN = PRDX1 PE = 1 SV = 1 | | | | | | |
| Phosphoglycerate mutase 1 OS = Homo sapiens | 29 | kDa | 0 | 11 | 0 | 0 |
| GN = PGAM1 PE = 1 SV = 2 | | | | | | |
| Pinin OS = Homo sapiens OX = 9606 GN = PNN PE = 1 | 82 | kDa | 0 | 6 | 2 | 4 |
| SV = 5 | | | | | | |
| Platelet-activating factor acetylhydrolase IB subunit | 47 | kDa | 0 | 4 | 0 | 0 |
| alpha OS = Homo sapiens OX = 9606 GN = PAFAH1B1 | | | | | | |
| PE = 1 SV = 2 | | | | | | |
| Poly [ADP-ribose] polymerase 1 OS = Homo sapiens | 113 | kDa | 0 | 30 | 0 | 0 |
| GN = PARP1 PE = 1 SV = 4 | | | | | | |
| Poly(U)-binding-splicing factor PUF60 (Fragment) | 57 | kDa | 0 | 3 | 0 | 0 |
| OS = Homo sapiens GN = PUF60 PE = 1 SV = 1 | | | | | | |
| Polymerase delta-interacting protein 3 OS = Homo sapiens | 48 | kDa | 0 | 6 | 0 | 0 |
| GN = POLDIP3 PE = 1 SV = 1 | | | | | | |
| Polymerase I and transcript release factor OS = Homo | 43 | kDa | 0 | 89 | 0 | 0 |
| sapiens GN = PTRF PE = 1 SV = 1 | | | | | | |
| Polyubiquitin-B OS = Homo sapiens GN = UBB PE = 1 | 17 | kDa | 0 | 111 | 0 | 0 |
| SV = 1 | | | | | | |
| POU domain, class 3, transcription factor 3 OS = Homo | 50 | kDa | 0 | 20 | 0 | 0 |
| sapiens GN = POU3F3 PE = 2 SV = 2 | | | | | | |
| PR domain zinc finger protein 8 OS = Homo sapiens | 72 | kDa | 0 | 2 | 0 | 0 |
| GN = PRDM8 PE = 1 SV = 3 | | | | | | |
| Prefoldin subunit 6 OS = Homo sapiens GN = PFDN6 | 15 | kDa | 0 | 3 | 0 | 0 |
| PE = 1 SV = 1 | | | | | | |
| Probable ATP-dependent RNA helicase DDX27 | 87 | kDa | 0 | 4 | 0 | 0 |
| OS = Homo sapiens GN = DDX27 PE = 1 SV = 1 | | | | | | |
| Probable global transcription activator SNF2L1 | 123 | kDa | 0 | 6 | 0 | 0 |
| OS = Homo sapiens OX = 9606 GN = SMARCA1 PE = 1 | | | | | | |
| SV = 2 | | | | | | |
| Probable maltase-glucoamylase 2 OS = Homo sapiens | 278 | kDa | 0 | 0 | 0 | 2 |
| OX = 9606 GN = MGAM2 PE = 2 SV = 3 | | | | | | |
| Procollagen galactosyltransferase 1 OS = Homo sapiens | 72 | kDa | 0 | 3 | 0 | 0 |
| GN = COLGALT1 PE = 1 SV = 1 | | | | | | |
| Procollagen-lysine, 2-oxoglutarate 5-dioxygenase 3 | 85 | kDa | 0 | 3 | 0 | 0 |
| OS = Homo sapiens GN = PLOD3 PE = 1 SV = 1 | | | | | | |
| Prolow-density lipoprotein receptor-related protein 1 | 505 | kDa | 0 | 2 | 0 | 0 |
| OS = Homo sapiens GN = LRP1 PE = 1 SV = 2 | | | | | | |
| Protein disulfide-isomerase OS = Homo sapiens | 53 | kDa | 0 | 30 | 0 | 0 |
| GN = P4HB PE = 1 SV = 2 | | | | | | |
| Protein GREB1 OS = Homo sapiens GN = GREB1 PE = 2 | 216 | kDa | 0 | 0 | 0 | 2 |
| SV = 1 | | | | | | |
| Protein kinase C delta-binding protein OS = Homo sapiens | 31 | kDa | 0 | 20 | 0 | 0 |
| GN = PRKCDBP PE = 1 SV = 1 | | | | | | |
| Protein MAK16 homolog OS = Homo sapiens | 35 | kDa | 0 | 2 | 0 | 0 |
| GN = MAK16 PE = 1 SV = 2 | | | | | | |
| Protein S100-A10 OS = Homo sapiens GN = S100A10 | 11 | kDa | 0 | 11 | 0 | 0 |
| PE = 1 SV = 2 | | | | | | |
| Protein S100-A13 OS = Homo sapiens GN = S100A13 | 11 | kDa | 0 | 5 | 0 | 0 |
| PE = 1 SV = 1 | | | | | | |
| Protein S100-A9 OS = Homo sapiens GN = S100A9 PE = 1 | 13 | kDa | 0 | 12 | 0 | 0 |
| SV = 1 | | | | | | |
| Protein-glutamine gamma-glutamyltransferase E | 77 | kDa | 0 | 2 | 0 | 0 |
| OS = Homo sapiens GN = TGM3 PE = 1 SV = 4 | | | | | | |
| Pumilio homolog 3 OS = Homo sapiens GN = PUM3 PE = 1 | 74 | kDa | 0 | 6 | 0 | 0 |
| SV = 3 | | | | | | |
| Raftlin OS = Homo sapiens GN = RFTN1 PE = 1 SV = 4 | 63 | kDa | 0 | 3 | 0 | 0 |
| Ras GTPase-activating-like protein IQGAP1 OS = Homo | 189 | kDa | 0 | 135 | 0 | 0 |
| sapiens GN = IQGAP1 PE = 1 SV = 1 | | | | | | |
| Ras-related protein Rab-10 OS = Homo sapiens | 23 | kDa | 0 | 13 | 0 | 0 |
| GN = RAB10 PE = 1 SV = 1 | | | | | | |
| Ras-related protein Rab-14 (Fragment) OS = Homo | 20 | kDa | 0 | 8 | 0 | 0 |
| sapiens GN = RAB14 PE = 1 SV = 1 | | | | | | |
| Ras-related protein Rab-2A OS = Homo sapiens | 24 | kDa | 0 | 5 | 0 | 1 |
| GN = RAB2A PE = 1 SV = 1 | | | | | | |
| Ras-related protein Ral-A OS = Homo sapiens GN = RALA | 24 | kDa | 0 | 2 | 0 | 0 |
| PE = 1 SV = 1 | | | | | | |
| Regulation of nuclear pre-mRNA domain-containing | 37 | kDa | 0 | 2 | 0 | 0 |
| protein 1B OS = Homo sapiens GN = RPRD1B PE = 1 SV = 1 | | | | | | |
| Replication protein A 32 kDa subunit OS = Homo sapiens | 29 | kDa | 0 | 3 | 0 | 1 |
| GN = RPA2 PE = 1 SV = 1 | | | | | | |
| Replication protein A 70 kDa DNA-binding subunit | 68 | kDa | 0 | 4 | 0 | 0 |
| OS = Homo sapiens GN = RPA1 PE = 1 SV = 2 | | | | | | |
| Reticulocalbin-1 OS = Homo sapiens GN = RCN1 PE = 1 | 39 | kDa | 0 | 7 | 0 | 0 |
| SV = 1 | | | | | | |
| Retinoic acid-induced protein 3 OS = Homo sapiens | 40 | kDa | 0 | 0 | 0 | 2 |
| OX = 9606 GN = GPRC5A PE = 1 SV = 2 | | | | | | |
| Rho GTPase-activating protein 1 OS = Homo sapiens | 50 | kDa | 0 | 8 | 0 | 0 |
| GN = ARHGAP1 PE = 1 SV = 1 | | | | | | |
| Ribosomal protein L19 OS = Homo sapiens GN = RPL19 | 23 | kDa | 0 | 21 | 0 | 0 |
| PE = 1 SV = 1 | | | | | | |
| Ribosome biogenesis protein BRX1 homolog OS = Homo | 41 | kDa | 0 | 5 | 0 | 3 |
| sapiens OX = 9606 GN = BRIX1 PE = 1 SV = 2 | | | | | | |
| Ribosome biogenesis protein WDR12 OS = Homo sapiens | 48 | kDa | 0 | 3 | 0 | 0 |
| GN = WDR12 PE = 1 SV = 2 | | | | | | |
| Ribosome biogenesis regulatory protein homolog | 41 | kDa | 0 | 11 | 0 | 0 |
| OS = Homo sapiens GN = RRS1 PE = 1 SV = 2 | | | | | | |
| Ribosome production factor 2 homolog OS = Homo | 36 | kDa | 0 | 3 | 0 | 0 |
| sapiens GN = RPF2 PE = 1 SV = 2 | | | | | | |
| RNA-binding protein 28 OS = Homo sapiens OX = 9606 | 86 | kDa | 0 | 2 | 0 | 2 |
| GN = RBM28 PE = 1 SV = 3 | | | | | | |
| RNA-binding protein 3 OS = Homo sapiens GN = RBM3 | 17 | kDa | 0 | 3 | 0 | 0 |
| PE = 1 SV = 1 | | | | | | |
| rRNA 2′-O-methyltransferase fibrillarin OS = Homo | 34 | kDa | 0 | 23 | 0 | 0 |
| sapiens GN = FBL PE = 1 SV = 2 | | | | | | |
| Selenoprotein H OS = Homo sapiens GN = C11orf31 PE = 1 | 13 | kDa | 0 | 2 | 0 | 0 |
| SV = 1 | | | | | | |
| Semaphorin-3C OS = Homo sapiens GN = SEMA3C PE = 2 | 85 | kDa | 0 | 3 | 0 | 0 |
| SV = 2 | | | | | | |
| Serine protease 23 OS = Homo sapiens OX = 9606 | 43 | kDa | 0 | 7 | 7 | 5 |
| GN = PRSS23 PE = 1 SV = 1 | | | | | | |
| Serine protease HTRA3 OS = Homo sapiens OX = 9606 | 49 | kDa | 0 | 3 | 3 | 0 |
| GN = HTRA3 PE = 1 SV = 2 | | | | | | |
| Serine/threonine-protein phosphatase 2A catalytic | 36 | kDa | 0 | 4 | 0 | 0 |
| subunit beta isoform OS = Homo sapiens GN = PPP2CB | | | | | | |
| PE = 1 SV = 1 | | | | | | |
| Serine/threonine-protein phosphatase PP1-beta catalytic | 37 | kDa | 0 | 22 | 0 | 0 |
| subunit OS = Homo sapiens GN = PPP1CB PE = 1 SV = 3 | | | | | | |
| Serpin B3 OS = Homo sapiens GN = SERPINB3 PE = 1 | 45 | kDa | 0 | 4 | 0 | 0 |
| SV = 2 | | | | | | |
| SH3 domain-binding protein 1 OS = Homo sapiens | 76 | kDa | 0 | 2 | 0 | 0 |
| GN = SH3BP1 PE = 1 SV = 3 | | | | | | |
| Signal peptidase complex subunit 1 OS = Homo sapiens | 12 | kDa | 0 | 2 | 0 | 0 |
| GN = SPCS1 PE = 1 SV = 4 | | | | | | |
| Signal peptidase complex subunit 3 OS = Homo sapiens | 20 | kDa | 0 | 5 | 0 | 0 |
| GN = SPCS3 PE = 1 SV = 1 | | | | | | |
| Signal recognition particle 14 kDa protein OS = Homo | 15 | kDa | 0 | 9 | 0 | 0 |
| sapiens GN = SRP14 PE = 1 SV = 2 | | | | | | |
| Signal-induced proliferation-associated 1-like protein 2 | 190 | kDa | 0 | 0 | 0 | 2 |
| OS = Homo sapiens OX = 9606 GN = SIPA1L2 PE = 1 SV = 2 | | | | | | |
| Single-stranded DNA-binding protein, mitochondrial | 17 | kDa | 0 | 24 | 0 | 0 |
| OS = Homo sapiens GN = SSBP1 PE = 1 SV = 1 | | | | | | |
| SNW domain-containing protein 1 OS = Homo sapiens | 61 | kDa | 0 | 4 | 0 | 0 |
| GN = SNW1 PE = 1 SV = 1 | | | | | | |
| Solute carrier family 2, facilitated glucose transporter | 54 | kDa | 0 | 9 | 0 | 0 |
| member 1 OS = Homo sapiens GN = SLC2A1 PE = 1 SV = 2 | | | | | | |
| SPARC (Fragment) OS = Homo sapiens GN = SPARC | 17 | kDa | 0 | 2 | 0 | 0 |
| PE = 1 SV = 1 | | | | | | |
| Spermatogenesis-associated serine-rich protein 2 | 60 | kDa | 0 | 2 | 0 | 0 |
| OS = Homo sapiens GN = SPATS2 PE = 1 SV = 1 | | | | | | |
| Sphingosine-1-phosphate lyase 1 OS = Homo sapiens | 64 | kDa | 0 | 2 | 0 | 0 |
| GN = SGPL1 PE = 1 SV = 3 | | | | | | |
| Splicing factor 3B subunit 2 OS = Homo sapiens | 98 | kDa | 0 | 7 | 0 | 0 |
| GN = SF3B2 PE = 1 SV = 1 | | | | | | |
| SRA stem-loop-interacting RNA-binding protein, | 14 | kDa | 0 | 2 | 0 | 0 |
| mitochondrial OS = Homo sapiens GN = SLIRP PE = 1 | | | | | | |
| SV = 1 | | | | | | |
| Structural maintenance of chromosomes protein 3 | 142 | kDa | 0 | 11 | 0 | 0 |
| OS = Homo sapiens GN = SMC3 PE = 1 SV = 2 | | | | | | |
| SWI/SNF-related matrix-associated actin-dependent | 122 | kDa | 0 | 17 | 0 | 0 |
| regulator of chromatin subfamily A member 5 OS = Homo | | | | | | |
| sapiens GN = SMARCA5 PE = 1 SV = 1 | | | | | | |
| SWI/SNF-related matrix-associated actin-dependent | 47 | kDa | 0 | 4 | 0 | 0 |
| regulator of chromatin subfamily E member 1 OS = Homo | | | | | | |
| sapiens OX = 9606 GN = SMARCE1 PE = 1 SV = 2 | | | | | | |
| SWISS-PROT: P01044-1 (Bos taurus) Isoform HMW of | 69 | kDa | 0 | 3 | 0 | 0 |
| Kininogen-1 precursor | | | | | | |
| SWISS-PROT: P02768-1 Tax_Id = 9606 | 69 | kDa | 0 | 0 | 5 | 13 |
| Gene_Symbol = ALB Isoform 1 of Serum albumin | | | | | | |
| precursor | | | | | | |
| SWISS-PROT: Q3SZR3 (Bos taurus) Alpha-1-acid | 23 | kDa | 0 | 2 | 0 | 0 |
| glycoprotein precursor | | | | | | |
| SWISS-PROT: Q3TTY5 Tax_Id = 10090 | 71 | kDa | 0 | 27 | 7 | 7 |
| Gene_Symbol = Krt2 Keratin, type II cytoskeletal 2 | | | | | | |
| epidermal | | | | | | |
| SWISS-PROT: Q9D312 Tax_Id = 10090 | 49 | kDa | 0 | 6 | 0 | 4 |
| Gene_Symbol = Krt20 Keratin, type I cytoskeletal 20 | | | | | | |
| SWISS-PROT: Q9QWL7 Tax_Id = 10090 | 48 | kDa | 0 | 54 | 0 | 16 |
| Gene_Symbol = Krt17 Keratin, type I cytoskeletal 17 | | | | | | |
| Synaptosomal-associated protein 23 OS = Homo sapiens | 23 | kDa | 0 | 6 | 0 | 0 |
| GN = SNAP23 PE = 1 SV = 1 | | | | | | |
| Tau-tubulin kinase 2 OS = Homo sapiens OX = 9606 | 182 | kDa | 0 | 0 | 0 | 0 |
| GN = TTBK2 PE = 1 SV = 1 | | | | | | |
| TBC1 domain family member 1 (Fragment) OS = Homo | 98 | kDa | 0 | 2 | 0 | 0 |
| sapiens GN = TBC1D1 PE = 1 SV = 3 | | | | | | |
| T-complex protein 1 subunit alpha OS = Homo sapiens | 60 | kDa | 0 | 98 | 0 | 0 |
| GN = TCP1 PE = 1 SV = 1 | | | | | | |
| Thyroid hormone receptor-associated protein 3 | 109 | kDa | 0 | 11 | 0 | 0 |
| OS = Homo sapiens GN = THRAP3 PE = 1 SV = 2 | | | | | | |
| Tight junction protein ZO-1 OS = Homo sapiens | 188 | kDa | 0 | 3 | 0 | 0 |
| GN = TJP1 PE = 1 SV = 1 | | | | | | |
| Tight junction protein ZO-2 OS = Homo sapiens | 141 | kDa | 0 | 3 | 0 | 0 |
| GN = TJP2 PE = 4 SV = 1 | | | | | | |
| Transcription factor A, mitochondrial OS = Homo sapiens | 29 | kDa | 0 | 14 | 1 | 1 |
| OX = 9606 GN = TFAM PE = 1 SV = 1 | | | | | | |
| Transcription factor SOX-3 OS = Homo sapiens | 45 | kDa | 0 | 2 | 0 | 0 |
| GN = SOX3 PE = 1 SV = 2 | | | | | | |
| Transforming growth factor-beta-induced protein ig-h3 | 75 | kDa | 0 | 670 | 73 | 58 |
| OS = Homo sapiens OX = 9606 GN = TGFBI PE = 1 SV = 1 | | | | | | |
| Translocator protein OS = Homo sapiens OX = 9606 | 19 | kDa | 0 | 0 | 0 | 3 |
| GN = TSPO PE = 1 SV = 3 | | | | | | |
| Transmembrane protein 165 OS = Homo sapiens | 35 | kDa | 0 | 5 | 0 | 1 |
| GN = TMEM165 PE = 1 SV = 1 | | | | | | |
| TREMBL: Q0V8M9; Q9TRI0 (Bos taurus) similar to | 100 | kDa | 0 | 7 | 0 | 0 |
| inter-alpha (globulin) inhibitor H3 isoform 2 | | | | | | |
| TREMBL: Q2KJC7; Q8HZM3 (Bos taurus) Periostin, | 87 | kDa | 0 | 1 | 12 | 5 |
| osteoblast specific factor | | | | | | |
| TREMBL: Q3SZH5 (Bos taurus) Similar to | 45 | kDa | 0 | 11 | 4 | 2 |
| Angiotensinogen | | | | | | |
| TREMBL: Q3T052;Q5EA67 (Bos taurus) Inter-alpha | 102 | kDa | 0 | 10 | 5 | 1 |
| (Globulin) inhibitor H4 | | | | | | |
| TREMBL: Q6ISB0 Keratin, hair, basic, 4 - Homo sapiens | 65 | kDa | 0 | 16 | 8 | 8 |
| (Human). | | | | | | |
| TREMBL: Q6NXH9 Tax_Id = 10090 | 59 | kDa | 0 | 114 | 7 | 10 |
| Gene_Symbol = Krt73 Keratin 73 | | | | | | |
| TRIO and F-actin-binding protein OS = Homo sapiens | 261 | kDa | 0 | 2 | 0 | 0 |
| OX = 9606 GN = TRIOBP PE = 1 SV = 3 | | | | | | |
| Tropomodulin-3 OS = Homo sapiens OX = 9606 | 40 | kDa | 0 | 47 | 21 | 13 |
| GN = TMOD3 PE = 1 SV = 1 | | | | | | |
| Tropomyosin 1 (Alpha), isoform CRA_f OS = Homo | 37 | kDa | 0 | 162 | 0 | 0 |
| sapiens GN = TPM1 PE = 1 SV = 1 | | | | | | |
| Tropomyosin 1 (Alpha), isoform CRA_m OS = Homo | 29 | kDa | 0 | 118 | 0 | 0 |
| sapiens GN = TPM1 PE = 1 SV = 1 | | | | | | |
| Tropomyosin alpha-3 chain OS = Homo sapiens | 33 | kDa | 0 | 112 | 0 | 0 |
| GN = TPM3 PE = 1 SV = 1 | | | | | | |
| Tubby-related protein 2 (Fragment) OS = Homo sapiens | 24 | kDa | 0 | 0 | 0 | 2 |
| OX = 9606 GN = TULP2 PE = 4 SV = 1 | | | | | | |
| Twinfilin-1 OS = Homo sapiens OX = 9606 GN = TWF1 | 40 | kDa | 0 | 1 | 1 | 4 |
| PE = 1 SV = 3 | | | | | | |
| Tyrosine-protein kinase OS = Homo sapiens GN = YES1 | 61 | kDa | 0 | 7 | 0 | 0 |
| PE = 1 SV = 1 | | | | | | |
| Tyrosine--tRNA ligase OS = Homo sapiens GN = YARS | 44 | kDa | 0 | 6 | 0 | 0 |
| PE = 1 SV = 1 | | | | | | |
| U1 small nuclear ribonucleoprotein A (Fragment) | 28 | kDa | 0 | 7 | 0 | 0 |
| OS = Homo sapiens GN = SNRPA PE = 1 SV = 1 | | | | | | |
| U3 small nucleolar ribonucleoprotein protein MPP10 | 79 | kDa | 0 | 6 | 0 | 0 |
| OS = Homo sapiens GN = MPHOSPH10 PE = 1 SV = 2 | | | | | | |
| U3 small nucleolar RNA-associated protein 14 homolog | 88 | kDa | 0 | 2 | 0 | 0 |
| A OS = Homo sapiens OX = 9606 GN = UTP14A PE = 1 | | | | | | |
| SV = 1 | | | | | | |
| U4/U6.U5 tri-snRNP-associated protein 1 OS = Homo | 90 | kDa | 0 | 4 | 0 | 0 |
| sapiens GN = SART1 PE = 1 SV = 1 | | | | | | |
| UAP56-interacting factor OS = Homo sapiens OX = 9606 | 36 | kDa | 0 | 3 | 0 | 0 |
| GN = FYTTD1 PE = 1 SV = 3 | | | | | | |
| Ubiquitin carboxyl-terminal hydrolase 24 OS = Homo | 294 | kDa | 0 | 0 | 0 | 2 |
| sapiens OX = 9606 GN = USP24 PE = 1 SV = 3 | | | | | | |
| Unconventional myosin-Id OS = Homo sapiens OX = 9606 | 116 | kDa | 0 | 0 | 2 | 0 |
| GN = MYO1D PE = 1 SV = 2 | | | | | | |
| Unconventional myosin-VI OS = Homo sapiens | 145 | kDa | 0 | 26 | 0 | 0 |
| GN = MYO6 PE = 1 SV = 1 | | | | | | |
| Unconventional myosin-XV OS = Homo sapiens | 395 | kDa | 0 | 0 | 2 | 0 |
| OX = 9606 GN = MYO15A PE = 1 SV = 2 | | | | | | |
| Urokinase-type plasminogen activator OS = Homo sapiens | 47 | kDa | 0 | 8 | 0 | 0 |
| GN = PLAU PE = 1 SV = 1 | | | | | | |
| UV excision repair protein RAD23 homolog B | 43 | kDa | 0 | 5 | 0 | 0 |
| OS = Homo sapiens GN = RAD23B PE = 1 SV = 1 | | | | | | |
| Vacuolar protein sorting-associated protein 26A | 38 | kDa | 0 | 5 | 0 | 3 |
| OS = Homo sapiens OX = 9606 GN = VPS26A PE = 1 SV = 2 | | | | | | |
| Very-long-chain enoyl-CoA reductase OS = Homo sapiens | 36 | kDa | 0 | 6 | 0 | 0 |
| GN = TECR PE = 1 SV = 1 | | | | | | |
| Vesicle-trafficking protein SEC22b OS = Homo sapiens | 25 | kDa | 0 | 6 | 0 | 0 |
| GN = SEC22B PE = 1 SV = 4 | | | | | | |
| V-type proton ATPase subunit B, brain isoform | 57 | kDa | 0 | 16 | 0 | 0 |
| OS = Homo sapiens GN = ATP6V1B2 PE = 1 SV = 3 | | | | | | |
| V-type proton ATPase subunit d 1 OS = Homo sapiens | 45 | kDa | 0 | 22 | 0 | 0 |
| GN = ATP6VOD1 PE = 1 SV = 1 | | | | | | |
| Zinc finger protein 469 OS = Homo sapiens OX = 9606 | 410 | kDa | 0 | 0 | 0 | 4 |
| GN = ZNF469 PE = 2 SV = 3 | | | | | | |

### Example 3—Proliferation of iPSCs on Amniotic Fluid Cell-Derived ECM

Induced pluripotent stem cells (iPSCs) were allowed to proliferate on an amniotic fluid cell-derived ECM (Matrix B) from Example 1 in culture using the following procedure: commercially available, cryopreserved iPSCs were thawed using a water bath at 37° C. Cell suspension was diluted into commercially available media for stem cell proliferation (Miltenyi Biotec MACS iPS Brew) and seeded onto the ECM at approximately 1,000 cells/cm² in a 6-well-plate with 2 mL of media/well. No Rock inhibitor was used. At day 1, the full volume of media was aspirated gently from cells in culture and replaced with fresh media. Every 24 hours, full media was replaced with fresh media. Once cells began to approach confluence (as determined by brightfield microscopy), cells were passage manually, using a sterile needle to cut large colonies into approximately 100 smaller colonies and then re-plate those by physically lifting them off the dish with the sterile needle and placing them on a fresh plate of the ECM. This procedure can be repeated indefinitely.

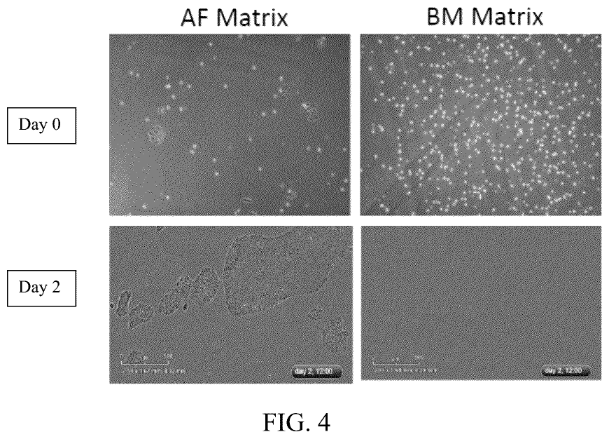

A photomicrograph showing Day 0 and Day 2 culture of iPSCs on amniotic fluid cell-derived ECM and a bone marrow cell-derived ECM is shown in FIG. 4.

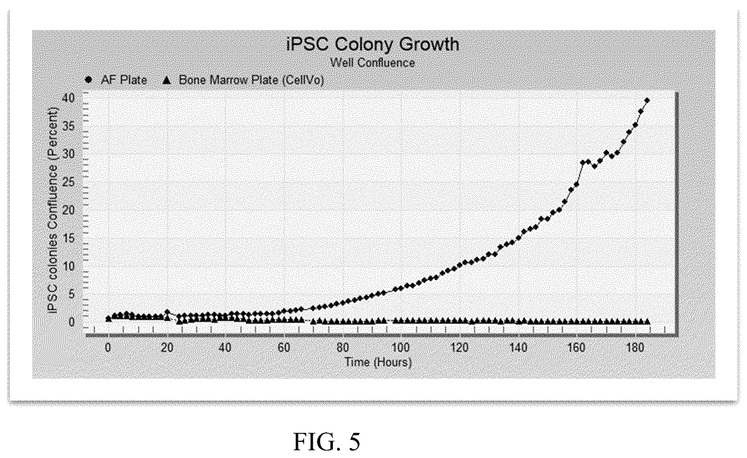

A plot of iPSC colony growth curves of iPSCs cultured in the presence of the amniotic fluid cell-derived ECM and a bone marrow cell-derived ECM is shown in FIG. 5.

As can be seen in FIG. 4 and FIG. 5, the iPSCs proliferated in culture in the presence of the amniotic fluid cell-derived ECM, whereas iPSCs cultured in the presence of a bone marrow cell-derived ECM had no growth.

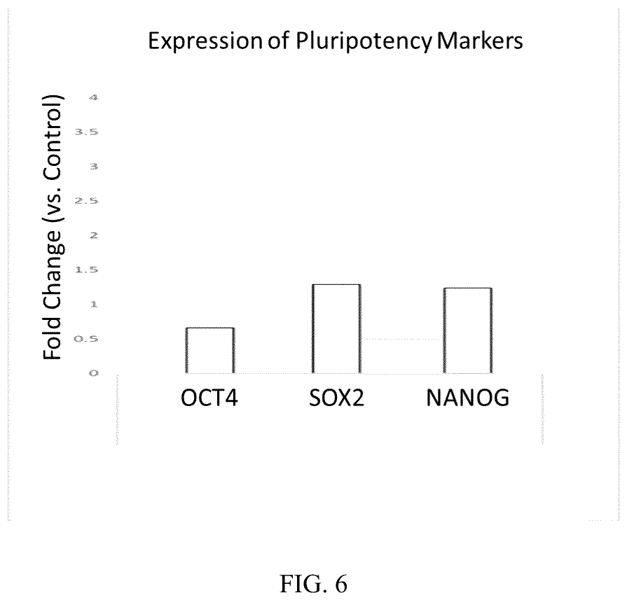

In another study, iPSCs were plated onto an amniotic fluid cell-derived ECM (AFC-ECM). Cells were passaged two times and then RNA was collected for gene expression analysis. Quantitative PCR was used to determine the gene expression. The iPSCs grown on the AFC-ECM maintained pluripotency during the course of the experiment and maintained expression of the core pluripotent transcription factors POU5F1 (OCT4), SOX2 and NANOG as shown in FIG. 6. The results from these studies suggest that the amniotic fluid cell-derived ECM supports pluripotent stem cell self-renewal and proliferation.

### Example 4—Proliferation of Various Cell Types on Amniotic Fluid Cell-Derived ECM

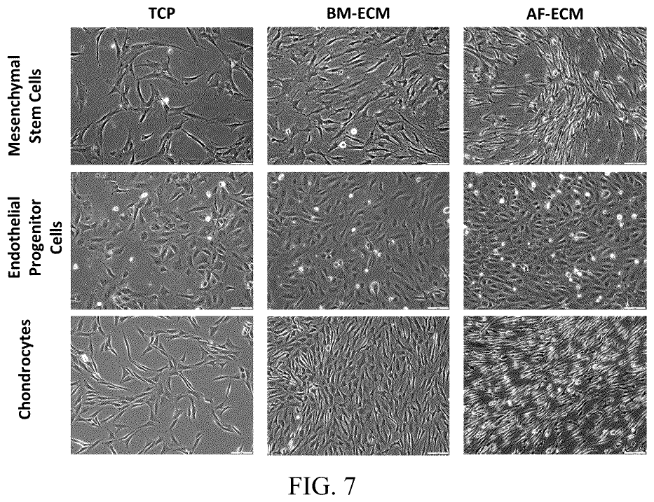

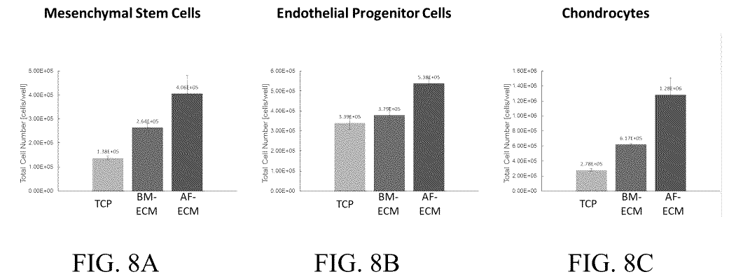

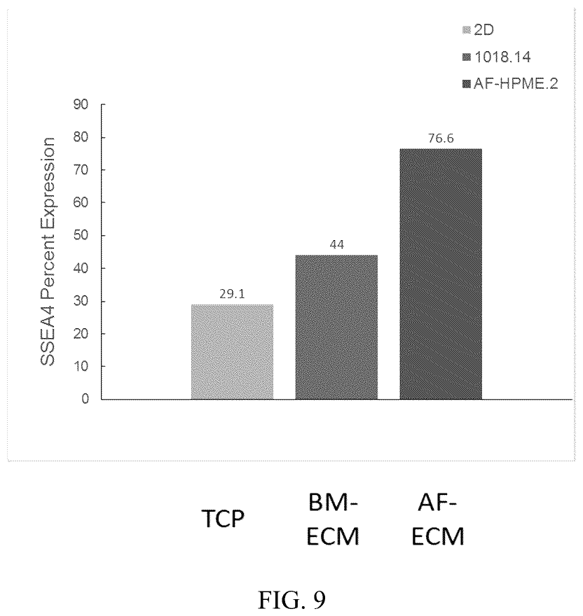

Mesenchymal stem cells (MSCs) obtained from bone marrow, endothelial progenitor cells (EPCs), and chondrocytes were seeded onto amniotic fluid cell-derived ECM (AF-ECM), bone marrow cell-derived ECM (BM-ECM), and tissue culture plastic (TCP) at equal seeding density in standard commercially available media. For MSCs and chondrocytes the media was alpha minimum essential media (aMEM) modified without phenol red, supplemented with 15% by volume fetal bovine serum, 1% antibiotic-antimycotic (anti-anti), and 1% GlutaMax. For EPCs, the media was Iscove's Modified Dulbecco's Medium (IMDM) Supplemented with 20% FBS, 1% anti-anti, 1% GlutaMax, and growth factors (EGF and FGF). Photomicrographs showing increased proliferation on the two ECMs relative to TCP are shown in FIG. 7. The proliferation of the cells after 4 days of culture is quantified in the bar graphs in FIG. 8a, FIG. 8b, and FIG. 8c. Surprisingly, the amniotic fluid cell-derived ECM supports increased proliferation of the MSCs (produced by bone marrow) relative to the bone marrow cell-derived ECM. Additionally, FIG. 9 shows the percentage of MSCs expressing SSEA-4 as measured by flow cytometry after 4 days of culture on each substrate. SSEA-4 expression is elevated after culture on amniotic fluid cell-derived ECM relative to the other substrates.

### Example 5—Differentiation of MSCs Cultured on Amniotic Fluid Cell-Derived ECM

Mesenchymal stem cells (MSCs) obtained from human bone marrow were seeded on amniotic fluid cell-derived ECM (AFC-ECM) at 6×10³ cells/cm² and cultured for 14 days in growth media.

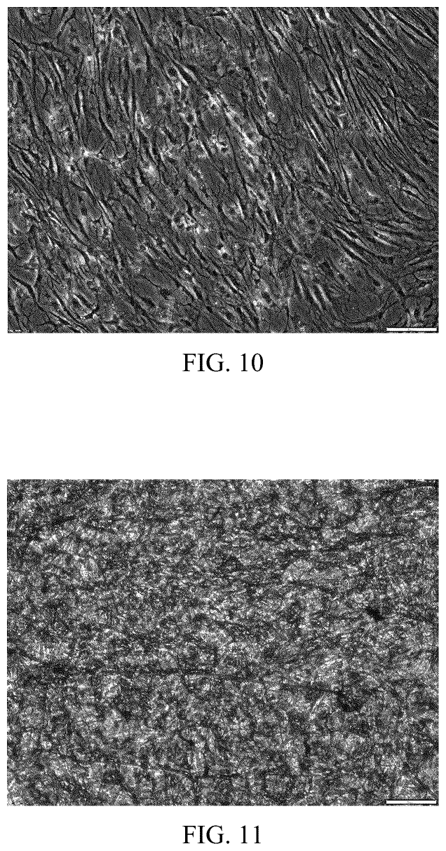

To assess the adipogenic differentiation efficiency of the cells, cultures were transferred at Day 7 to adipogenic media (DMEM containing 10% FBS, 0.5 mM IBMX, 10⁻⁶ M dexamethasone, 10 μM insulin, and 200 μM indomethacin), maintained for an additional 10 days, fixed with 10% formalin for 1 hour at room temperature, and then stained with Oil Red O. Adipogenesis is verified by staining the differentiated cells with the Oil Red O. The Oil Red O stains the lipid droplets (red staining) formed within the cells following the differentiation of the MSCs to adipocytes in culture. Photomicrographs showing adipogenic differentiation of the MSCs on the AFC-ECM is shown in FIG. 10.

To assess osteogenic differentiation efficiency, the cultures were transferred to osteoblast differentiation media (growth media supplemented with 10⁻⁷ M dexamethasone (Sigma-Aldrich, St. Louis, MO) and 10⁻⁴ M L-ascorbate-2-phosphate (Wako Chemicals, Richmond, VA), maintained an additional 25 days, fixed with 10% formalin for 1 hour at room temperature, and then stained with 1% Silver Nitrate (Von Kossa Staining). Osteogenesis is verified by staining the differentiated cells with 1% Silver Nitrate. The Silver Nitrate stains the minerals (dark staining) deposited following the differentiation of the MSCs to osteoblasts in culture. Photomicrographs showing osteogenic differentiation of the MSCs on the AFC-ECM is shown in FIG. 11. This study demonstrates that stem cells can be induced to differentiate into differentiated cell types in culture on an amniotic fluid cell-derived ECM.

1. A cell-derived extracellular matrix (ECM) derived in vitro from cells isolated from amniotic fluid.

2. The cell-derived ECM of claim 1, wherein the ECM comprises laminin, collagen alpha-1 (XVIII), basement membrane-specific heparan sulfate proteoglycan core protein, agrin, vimentin, and collagen alpha-2 (IV), and/or isoforms thereof.

3. The cell-derived ECM of claim 2, wherein the isoform of collagen alpha-1 (XVIII) is isoform 2, and/or wherein the isoform of agrin is isoform 6.

4. The cell derived ECM of any one of claim 2 or 3, wherein the cell-derived ECM further comprises fibronectin and/or an isoform thereof.

5. The cell-derived ECM of any one of claims 1 to 4, wherein the cell-derived ECM does not contain decorin, perlecan, and/or collagen (III).

6. The cell-derived ECM of any one of claims 1 to 5, wherein the cells isolated from amniotic fluid comprise stem cells.

7. The cell-derived ECM of any one of claims 1 to 6, wherein the cell-derived ECM is decellularized.

8. A method of proliferating adherent cells in culture, the method comprising culturing the adherent cells in the presence of a cell-derived extracellular matrix (ECM) in a culture media thereby proliferating the adherent cells, wherein the cell-derived ECM is derived in vitro from cells isolated from amniotic fluid.

9. The method of claim 8, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof.

10. The method of claim 9, wherein the isoform of collagen alpha-1 (XVIII) is isoform 2, and/or wherein the isoform of agrin is isoform 6.

11. The method of any one of claim 9 or 10, wherein the cell-derived ECM further comprises fibronectin and/or an isoform thereof.

12. The method of any one of claims 8 to 11, wherein the cell-derived ECM does not contain decorin, perlecan, and/or collagen (III).

13. The method of any one of claims 8 to 12, wherein the cells isolated from amniotic fluid comprise stem cells.

14. The method of any one of claims 8 to 13, wherein the cell-derived ECM is decellularized prior to contact with the adherent stem cells.

15. The method of any one of claims 8 to 14, wherein the adherent cells comprise mammalian adherent cells.

16. The method of any one of claims 8 to 15, wherein the adherent cells comprise stem cells, somatic cells, progenitor cells, mature cells, or cells from multiple germ layers.

17. The method of claim 16, wherein the adherent cells comprise stem cells.

18. The method of claim 17, wherein the stem cells are maintained in an undifferentiated state.

19. The method any one of claim 17 or 18, wherein the stem cells comprise pluripotent stem cells (PSCs).

20. The method of claim 19, wherein the PSCs comprise induced PSCs (iPSC).

21. The method of claim 19, wherein the PSCs comprise embryonic stem cells(ES).

22. The method of any one of claim 17 or 18, wherein the stem cells comprise mesenchymal stem cells (MSCs).

23. The method of claim 22, wherein the MSCs are obtained from bone marrow.

24. The method of claim 16, wherein the adherent cells comprise progenitor cells.

25. The method of claim 24, wherein the progenitor cells comprise endothelial progenitor cells.

26. The method of claim 16, wherein the adherent cells comprise mature cells.

27. The method of claim 26, wherein the mature cells comprise chondrocytes.

28. A method of inducing differentiation of stem cells into differentiated cell types, the method comprising culturing the stem cells in the presence of a cell-derived extracellular matrix (ECM) in a differentiation media, wherein the cell-derived ECM is derived in vitro from cells isolated from amniotic fluid.

29. The method of claim 28, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof.

30. The method of claim 29, wherein the isoform of collagen alpha-1 (XVIII) is isoform 2, and/or wherein the isoform of agrin is isoform 6.

31. The method of any one of claim 29 or 30, wherein the cell-derived ECM further comprises fibronectin and/or an isoform thereof.

32. The method of any one of claims 28 to 31, wherein the cell-derived ECM does not contain decorin, perlecan, and/or collagen (III).

33. The method of any one of claims 28 to 32, wherein the cells isolated from amniotic fluid comprise stem cells.

34. The method of any one of claims 28 to 33, wherein the stem cells comprise pluripotent stem cells (PSCs).

35. The method of claim 34, wherein the PSCs comprise induced PSCs (iPSC).

36. The method of claim 34, wherein the PSCs comprise embryonic stem cells(ES).

37. The method of any one of claims 28 to 33, wherein the stem cells comprise mesenchymal stem cells (MSCs).

38. The method of claim 22, wherein the MSCs are obtained from bone marrow.

39. The method of any one of claim 37 or 38, wherein the differentiated cell types comprise adipocytes, osteoblasts, chondrocytes, or myocytes.

40. A method of producing a cell-derived extracellular matrix (ECM) in vitro, the method comprising:
(a) isolating cells from amniotic fluid;
(b) seeding the isolated cells onto a cell culture container or onto a cell culture container coated with a substrate;
(c) adding a culture media to the cell culture container; and
(d) culturing the cells, thereby producing a cell-derived ECM; and
(e) optionally decellularizing the cell-derived ECM.

41. The method of claim 40, wherein the isolated cells from the amniotic fluid comprise stem cells.

42. The method of claim 40 or 41, wherein the substrate is fibronectin.

43. The method of any one of claims 40 to 42, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof.

44. The method of claim 43, wherein the isoform of collagen alpha-1 (XVIII) is isoform 2, and/or wherein the isoform of agrin is isoform 6.

45. The method of any one of claim 43 or 44, wherein the cell-derived ECM further comprises fibronectin and/or an isoform thereof.

46. The method of any one of claims 40 to 45, wherein the cell-derived ECM does not contain decorin, perlecan, and/or collagen (III).

47. An amniotic fluid cell-derived extracellular matrix (AFC-ECM) produced in vitro by the method comprising:
(a) isolating cells from amniotic fluid,
(b) seeding the isolated cells onto a cell culture container or onto a cell culture container coated with a substrate,
(c) adding a culture media to the cell culture container, and
(d) culturing the cells, thereby producing the AFC-ECM, and
(e) optionally decellularizing the AFC-ECM.

48. The method of claim 47, wherein the isolated cells from the amniotic fluid comprise stem cells.

49. The method of claim 47 or 48, wherein the substrate is fibronectin.

50. The method of any one of claims 47 to 49, wherein the cell-derived ECM comprises laminin, collagen alpha-1 (XVIII), basement membrane-specific heparan sulfate proteoglycan core protein, agrin, vimentin, and collagen alpha-2 (IV), and/or isoforms thereof.

51. The method of any claim 50, wherein the isoform of collagen alpha-1 (XVIII) is isoform 2, and/or wherein the isoform of agrin is isoform 6.

52. The method of any one of claim 50 or 51, wherein the cell-derived ECM further comprises fibronectin and/or an isoform thereof.

53. The method of any one of claims 47 to 52, wherein the cell-derived ECM does not contain decorin. perlecan, and/or collagen (III).
